(19) United States
(12) Patent Application Publication
(10) Pub. No.: US 2021/0395779 A1
(43) Pub. Date: Dec. 23, 2021

(54) TWO-GENE VECTORS FOR GENERATING CAR-T CELLS AND USES THEREOF

(71) Applicant: MediSix Therapeutics Pte Ltd., Singapore (SG)

(72) Inventors: Rizal Ismail, Singapore (SG); Yunqin Lee, Singapore (SG); Murray Robinson, Singapore (SG); Ying Xim Tan, Singapore (SG)

(21) Appl. No.: 17/291,580

(22) PCT Filed: Nov. 14, 2019
(86) PCT No.: PCT/US2019/061549

§ 371 Date: May 5, 2021

Related U.S. Application Data
(60) Provisional application No. 62/767,069, filed on Nov. 14, 2018.

(51) Int. Cl. C12N 15/86; C07K 16/28; A61K 35/17; C12N 5/0783

(52) U.S. Cl. CPC C12N 15/86; C07K 16/2803; A61K 35/17; C12N 5/0636; C12N 2830/20; A61K 38/00; C12N 2840/203; C07K 2319/33; C07K 2317/76; C07K 2319/30; C12N 2740/15043

(57) ABSTRACT

The present invention provides two-gene retroviral vector compositions comprising polynucleotides encoding an anti-CD7 chimeric activating receptor (CAR) and polynucleotides encoding an anti-CD7 protein expression blocker. Also provided are methods of producing and methods of using such compositions in cancer therapy.

### CROSS-REFERENCE TO RELATED APPLICATIONS

This application claims benefit to U.S. Provisional Application No. 62/767,069 filed Nov. 14, 2018, the disclosure in its entirety is herein incorporated by reference.

### REFERENCE TO A SEQUENCE LISTING

The instant application contains a Sequence Listing which has been submitted electronically in ASCII format and is hereby incorporated by reference in its entirety. Said Sequence Listing was created on Nov. 14, 2019, and is entitled “119419-5007-WO-Sequence-Listing_ST25.txt” which is 139,264 bytes in size.

### BACKGROUND OF THE INVENTION

Chimeric antigen receptors (CARs) can redirect immune cells to specifically recognize and kill tumor cells. CARs are artificial multi-molecular proteins constituted by a single-chain variable region (scFv) of an antibody linked to a signaling molecule via a transmembrane domain. When the scFv ligates its cognate antigen, signal transduction is triggered, resulting in tumor cell killing by CAR-expressing cytotoxic T lymphocytes (Eshhar Z, Waks T, et al. PNAS USA. 90(2):720-724, 1993; Geiger T L, et al. J Immunol. 162(10):5931-5939, 1999; Brentjens R J, et al. Nat Med. 9(3):279-286, 2003; Cooper L J, et al. Blood 101(4):1637-1644, 2003; Imai C, et al. Leukemia. 18:676-684, 2004). Clinical trials with CAR-expressing autologous T lymphocytes have shown positive responses in patients with B-cell refractory leukemia and lymphoma (see, e.g., Till B G, et al. Blood 119(17):3940-3950, 2012; Maude S L, et al. N Engl J Med. 371(16):1507-1517, 2014).

The development of CAR technology to target T cell malignancies has lagged far behind the progress made for their B-cell counterparts. Novel therapies for T-cell malignancies are needed but progress to date has been slow. In particular, effective immunotherapeutic options are lacking and treatment of T-cell acute lymphocytic leukemia (T-ALL) relies on intensive chemotherapy and hematopoietic stem cell transplant. Despite aggressive treatment regimes associated with significant morbidity, results with these approaches are far from satisfactory.

CAR-T cells have recently been developed in which the target antigen of the CAR-T is itself expressed in the CAR-T cell (Png et al., Blood, 2017, 1(25):2348-2360, WO 2018/098306). To avoid self-killing (e.g., fratricide), the CAR-T cells also express a PEBL that serves to reduce the expression of the target antigen on the cell surface of the CAR-T. To produce viable CAR-T cells, first a protein expression blocker (PEBL) protein was expressed to bind and sequester the target protein prior to the subsequent expression of the CAR. Due to the pre-existing presence of the target antigen on the cell surface of the resulting engineered T cells, simultaneous expression of the CAR and the PEBL resulted in fratricide. In particular, the pre-existing cell surface target antigens were not susceptible to sequestration by the newly expressed PEBL proteins, and are recognized and targeted by the newly expressed CAR proteins.

An alternative to simultaneous expression can be sequential expression. However, sequential expression of a PEBL and then a CAR in a T-cell creates several challenges for the clinical implementation of PEBL CAR-T cells. First, sequential engineering of the T cells requires the separate manufacture and administration of distinct viral vectors, one for the PEBL and a second for the CAR. This increases cost and time, as well as the complexity of experimental manipulation to produce the engineered CAR-T cells. In addition, sequential engineering of the T cells results in a complex mix of engineered cells in the final clinical product, creating challenges with product characterization, uniformity and efficacy. Because only a fraction of the T cells integrates the introduced gene at each engineering step, the final product (the engineered T cells) will comprise some cells that only received the PEBL gene, some cells that only received the CAR gene, and some cells that received both genes.

In sum, there is a significant unmet need for new therapeutic options for patients with T-cell malignancies. Additionally, there is a need for methods for producing an engineered CAR-T cell and eliminating CAR-mediated self-killing or fratricide of the T cell.

### SUMMARY OF THE INVENTION

In some aspects, the present invention provides a bicistronic retroviral vector comprising: (a) a first polynucleotide encoding an anti-CD7 chimeric antigen receptor (CAR) comprising at least 90% sequence identity to the amino acid sequence of any one of SEQ ID NOS:28-31; (b) a second polynucleotide encoding an Internal Ribosome Entry Site (IRES) or a ribosomal codon skipping site; and (c)) a third polynucleotide encoding an anti-CD7 protein expression blocker (PEBL) comprising at least 90% sequence identity to the amino acid sequence of SEQ ID NOS:24-27, wherein the first polynucleotide is operably linked the second polynucleotide which is operably linked the third polynucleotide.

In some embodiments, the anti-CD7 CAR comprises the amino acid sequence of any one of SEQ ID NOS:28-31.

In some embodiments, the anti-CD7 PEBL comprises the amino acid sequence of any one of SEQ ID NOS:24-27.

In some embodiments, the anti-CD7 CAR comprises the amino acid sequence of SEQ ID NO:29 and the anti-CD7 PEBL comprises the amino acid sequence of SEQ ID NO:25.

In some embodiments, the anti-CD7 CAR comprises the amino acid sequence of SEQ ID NO:31 and the anti-CD7 PEBL comprises the amino acid sequence of SEQ ID NO:27.

In some embodiments, the IRES is derived from Encephalomyocarditis virus (EMCV) or an Enterovirus.

In some embodiments, the ribosomal codon skipping site comprises a 2A self-cleaving peptide. In some embodiments, the 2A self-cleaving peptide is selected from the group consisting of a F2A peptide (foot-and-mouth disease virus 2A peptide), an E2A peptide (equine rhinitis A virus 2A peptide), a P2A peptide (porcine teschovirus-1 2A peptide), and a T2A peptide (thosea asigna virus 2A).

In some embodiments, the bicistronic retroviral vector comprises at least 90% sequence identity to the nucleic acid sequence of SEQ ID NO:12.

In some embodiments, the bicistronic retroviral vector comprises least 90% sequence identity to the nucleic acid sequence of SEQ ID NO:13. some embodiments, the bicistronic retroviral vector comprises the nucleic acid sequence of SEQ ID NO:13.

In some embodiments, the bicistronic retroviral vector comprises further comprises a promoter element.

In some embodiments, the promoter element is selected from the group consisting of a CMV promoter, EF1α promoter, EFS promoter, MSCV promoter, and PGK promoter.

In some embodiments, the promoter element comprises at least 90% sequence identity to the nucleic acid sequence of any one selected from the group consisting of SEQ ID NOS:6-10.

In some embodiments, the promoter element comprises the nucleic acid sequence of any one selected from the group consisting of SEQ ID NOS:6-10.

In some embodiments, the bicistronic retroviral vector comprises at least 90% sequence identity to the nucleic acid sequence of any one selected from the group consisting of SEQ ID NOS:14-16.

In some embodiments, the bicistronic retroviral vector comprises the nucleic acid sequence of any one selected from the group consisting of SEQ ID NOS:14-16.

In some embodiments, the retroviral vector is a lentiviral vector.

In some aspects, provided herein is an engineered immune cell comprising any one of the bicistronic retroviral vectors outlined herein.

In some embodiments, the engineered immune cell is an allogeneic T cell. In some embodiments, the engineered immune cell is an autologous T cell.

In some embodiments, the engineered immune cell has reduced CD7 surface expression compared to a corresponding immune cell and expresses the anti-CD7 CAR.

In some aspects, provided herein is a pharmaceutical composition comprising any of the engineered immune cells described herein and a pharmaceutically effective carrier.

In some aspects, provided herein is a method of treating a cancer in a subject comprising administering a therapeutically effective amount of any of the engineered immune cells described herein or a pharmaceutical composition thereof.

In some aspects, provided herein is a method of producing an engineered immune cell comprising transducing an immune cell with any one of the bicistronic retroviral vectors described herein and recovering the engineered immune cell.

In some embodiments, the immune cell is selected from the group consisting of a peripheral blood mononuclear cell, an isolated CD4+ T cell, an isolated CD8+ T cell, and an isolated CD3+ T cell.

In some embodiments, the engineered immune cell has reduced CD7 surface expression compared to a corresponding immune cell and expresses the anti-CD7 CAR.

In some aspects, provided herein is a recombinant retroviral vector comprising: (a) a first promoter element operably linked to a first polynucleotide encoding an anti-CD7 chimeric antigen receptor (CAR) comprising at least 90% sequence identity to the amino acid sequence of SEQ ID NO:28 or SEQ ID NO:30; and (b) a second promoter element operably linked to a second polynucleotide encoding an anti-CD7 protein expression blocker (PEBL) comprising at least 90% sequence identity to the amino acid sequence of SEQ ID NO:24 or SEQ ID NO:26.

In some embodiments, the anti-CD7 CAR comprises the amino acid sequence of SEQ ID NO:28 or SEQ ID NO:30.

In some embodiments, the anti-CD7 PEBL comprises the amino acid sequence of SEQ ID NO:24 or SEQ ID NO:26.

In some embodiments, the first promoter element and/or the second promoter element are selected from the group consisting of a CMV promoter, EF1α promoter, EFS promoter, MSCV promoter, and PGK promoter.

In some embodiments, the first promoter element and/or the second promoter element comprise at least 90% sequence identity to the nucleic acid sequence of any one selected from the group consisting of SEQ ID NOS:6-10.

In some embodiments, the first promoter element and/or the second promoter element comprise the nucleic acid sequence of any one selected from the group consisting of SEQ ID NOS:6-10.

In some embodiments, the first promoter and the second promoter share less than 95% sequence identity.

In some embodiments, the first promoter element operably linked to the first polynucleotide is 5′ of the second promoter element operably linked to the second polynucleotide.

In some embodiments, the second promoter element operably linked to the second polynucleotide is 5′ of the first promoter element operably linked to the first polynucleotide.

In some embodiments, the recombinant retroviral vector comprises at least 90% sequence identity to the nucleic acid sequence of any one selected from the group consisting of SEQ ID NOS:18-23.

In some embodiments, the retroviral vector is a lentiviral vector.

Also provided is an engineered immune cell comprising any one of the recombinant retroviral vectors described herein.

In some embodiments, the engineered immune cell is an allogenic T cell. In some embodiments, the engineered immune cell is an autologous T cell.

In some embodiments, the engineered immune cell has reduced CD7 surface expression compared to a corresponding immune cell and expresses the anti-CD7 CAR.

In some aspects, provided herein is a pharmaceutical composition comprising any of the engineered immune cells described herein and a pharmaceutically effective carrier.

In some aspects, provided herein is a method of treating a cancer in a subject comprising administering a therapeutically effective amount of any of the engineered immune cells described herein or a pharmaceutical composition thereof.

In some aspects, provided herein is a method of producing an engineered immune cell comprising transducing an immune cell with any one of the recombinant retroviral vectors described herein and recovering the engineered immune cell.

In some embodiments, the immune cell is selected from the group consisting of a peripheral blood mononuclear cell, an isolated CD4+ T cell, an isolated CD8+ T cell, and an isolated CD3+ T cell.

In some embodiments, the engineered immune cell has reduced CD7 surface expression compared to a corresponding immune cell and expresses the anti-CD7 CAR.

### BRIEF DESCRIPTION OF THE DRAWINGS

FIG. 1A and FIG. 1B show expression of CAR and PEBL by transduced primary T cells according to flow cytometry (FIG. 1A) and Western blot (FIG. 1). Primary T cells were transduced with the indicated retroviruses (e.g., PEBL; CAR; PEBL and CAR sequentially; PEBL-IRES-CAR; and CAR-P2A-PEBL) and analyzed by flow cytometry for CD7 and CAR expression. Cell lysates from primary T cells transduced with the indicated retroviruses were analyzed by Western blot for β-actin, Myc-tagged PEBL, CAR and endogenous CD3ζ expression.

FIG. 2A-FIG. 2F provide illustrative schematic diagrams of bicistronic promoter 1-CAR-promoter 2-PEBL lentiviral constructs. FIG. 2A depicts a schematic of an exemplary dual promoter construct comprising a MSCV-promoter-anti-human CD7 (TH69) CAR-EFS promoter-anti-human CD7 (TH69) PEBL, such as the one of SEQ ID NO:19. FIG. 2B depicts a schematic of an exemplary dual promoter construct comprising a MSCV promoter-anti-human CD7 (TH69) CAR-EF1a promoter-anti-human CD7 (TH69) PEBL, such as the one of SEQ ID NO:18. FIG. 2C depicts a schematic of an exemplary dual promoter construct comprising a PGK promoter-anti-human CD7 (TH69) CAR-EFS promoter-anti-human CD7 (TH69) PEBL, such as the one of SEQ ID NO:23. FIG. 2D depicts a schematic of an exemplary dual promoter construct comprising a PGK promoter-anti-human CD7 (TH69) CAR-EF1a promoter-anti-human CD7 (TH69) PEBL, such as the one of SEQ ID NO:22. FIG. 2E depicts a schematic of an exemplary dual promoter construct comprising a PGK promoter-anti-human CD7 (3A1F) CAR-EF1a promoter-anti-human CD7 (TH69) PEBL, such as the one of SEQ ID NO:20. FIG. 2F depicts a schematic of an exemplary dual promoter construct comprising a PGK promoter-anti-human CD7 (TH69) CAR-EF1a promoter-anti-human CD7 (3A1F) PEBL, such as the one of SEQ ID NO:21.

FIG. 3 shows expression of CAR and CD7 by transduced primary T cells according to flow cytometry. Primary T cells were transduced with the indicated dual-promoter lentiviruses and analyzed by flow cytometry at 5 days and 14 days post transduction.

FIG. 4A-FIG. 4C provide illustrative schematic diagrams of bicistronic CAR-P2A-PEBL lentiviral constructs. FIG. 4A depicts a schematic of an exemplary bicistronic construct comprising an MSCV promoter-anti-human CD7 (TH69) CAR-P2A-anti-human CD7 (TH69) PEBL, such as the one of SEQ ID NO:14. FIG. 4B depicts a schematic of an exemplary bicistronic construct comprising an EF1a promoter-anti-human CD7 (TH69) CAR-P2A-anti-human CD7 (TH69) PEBL, such as the one of SEQ ID NO:15. FIG. 4C depicts a schematic of an exemplary bicistronic construct comprising an EFS promoter-anti-human CD7 (TH69) CAR-P2A-anti-human CD7 (TH69) PEBL, such as the one of SEQ ID NO:16.

FIG. 5 shows expression of CAR and CD7 by primary T cells transduced with MSCV-CD7CAR-P2A-CD7PEBL lentivirus and analyzed by flow cytometry at 3 days, 6 days, and 9 days post transduction.

FIG. 6 shows expression of CAR and CD7 by transduced primary T cells according to flow cytometry. Primary T cells were transduced with the indicated bicistronic CD7CAR-P2A-CD7PEBL and CD19CAR lentiviruses and analyzed by flow cytometry at 5 days and 14 days post transduction.

FIG. 7A and FIG. 7B show expression of CAR and PEBL by transduced primary T cells according to flow cytometry (FIG. 7A) and Western blot (FIG. 7B). Primary T cells were transduced with the two independently produced lots of MSCV-CD7CAR-P2A-CD7PEBL lentivirus and analyzed by flow cytometry for CD7 and CAR expression. Cell lysates from transduced cells were analyzed by Western blot for β-actin, Myc-tagged PEBL, CAR and endogenous CD3ζ expression.

FIG. 8 shows expression of CAR and CD7 by transduced primary T cells according to flow cytometry. Bulk PBMCs, CD4⁺ and CD8⁺ positively-selected T cells, and CD3⁺ positively-selected T cells were activated with either Dynabeads or TransAct and transduced with MSCV-CD7CAR-P2A-CD7PEBL lentivirus. Cells were analyzed by flow cytometry at 4 days, 7 days, and 10 days post transduction.

FIG. 9A shows expression of CAR and CD7 by primary T cells transduced with MSCV-CD7CAR-P2A-CD7PEBL lentivirus and cultured in serum-free TexMACS medium, or TexMACS medium supplemented with 3% human AB serum. FIG. 9B shows the total fold expansion of transduced cells at 11 days post activation (mean±SEM of biological replicates).

FIG. 10A and FIG. 10B show percentage of CAR+ T cells (FIG. 10A) and total fold expansion (FIG. 10B) of transduced cells at 11 days post activation (mean±SEM of biological replicates). Primary T cells were cultured in serum-free TexMACS medium and transduced with MSCV-CD7CAR-P2A-CD7PEBL lentivirus at 1, 2, 3, or 4 days post activation.

FIG. 11 shows expression of CAR and CD7 by transduced primary T cells according to flow cytometry. CD4⁺ and CD8⁺ positively-selected T cells were activated with TransAct and transduced with MSCV-CD7CAR-P2A-CD7PEBL lentivirus at the indicated multiplicity of infection (MOI). Cells were analyzed by flow cytometry at 3 days and 9 days post transduction.

FIG. 12A and FIG. 12B show percentage of CAR+ T cells (FIG. 12A) and transgene vector copy number (VCN) (FIG. 12B) of transduced cells at 11 days post activation (mean of biological duplicates). Primary T cells were cultured in serum-free TexMACS medium and transduced with MSCV-CD7CAR-P2A-CD7PEBL lentivirus at MOI 3, 5, or 10. T cells were analyzed by flow cytometry for CAR expression. Genomic DNA was extracted from transduced cells to determine transgene VCN by RT-qPCR analysis.

FIG. 13A-FIG. 13E show expression of various surface markers on primary T cells transduced with MSCV-CD7CAR-P2A-CD7PEBL lentivirus at 11 days post activation. Transduced cells were analyzed by flow cytometry for CAR and CD7 (FIG. 13A), CD3 and CD14/CD19/CD56 (FIG. 13B), CD4 and CD8 (FIG. 13C), CD45RO and CCR7 (FIG. 13D), and PD-1 and Tim-3 (FIG. 13E). The triplicate analyses are of primary T cells from 3 unique donors transduced with MSCV-CD7CAR-P2A-CD7PEBL lentivirus at MOI 10.

FIG. 14A and FIG. 14B show functional response of PEBL-CAR-T cells, generated with MSCV-CD7CAR-P2A-CD7PEBL lentivirus, to CD7+ Jurkat cells and CD7− Nalm6 cells by IFNγ secretion (FIG. 14A) and cytotoxicity (FIG. 14B). IFNγ secretion was measured in culture supernatants of PEBL-CAR-T cells co-cultured with Jurkat or Nalm6 cells at the indicated E:T ratios for 24 h (mean±SD of technical replicates). Cytolytic activity of PEBL-CAR T cells was measured after a 4 h co-culture with Jurkat or Nalm6 cells at the indicated E:T ratios (mean±SD of technical replicates).

FIG. 15 depicts the nucleic acid sequence of CPPT-CMV-MCS-PGK-GFP-WPRE (SEQ ID NO:1). The CPPT is in bold, CMV promoter is single underlined, PGK promoter is double underlined, GFP is bold/single underlined, and the WPRE element is bold/double underlined.

FIG. 16 depicts the nucleic acid sequence of an exemplary anti-human CD7 PEBL based on the antibody TH69 (SEQ ID NO:2). The CD8 signal peptide starts at position 1, the anti-CD7 VL domain is in bold, the linker between the VL and VH domains is single underlined, the anti-CD7 VH domain is in bold/single underlined, the myc-KDEL peptide is double underlined, and a stop codon ends the sequence.

FIG. 17 depicts the nucleic acid sequence of an exemplary anti-human CD7 PEBL based on the antibody 3A1F (SEQ ID NO:3). The CD8 signal peptide starts at position 1, the anti-CD7 VL domain is in bold, the linker between the VL and VH domains is single underlined, the anti-CD7 VH domain is in bold/single underlined, the myc-KDEL peptide is double underlined, and a stop codon ends the sequence.

FIG. 18 depicts the nucleic acid sequence of an exemplary anti-human CD7 CAR based on the antibody TH69 (SEQ ID NO:4). The CD8 signal peptide starts at position 1, the anti-CD7 VL domain is in bold, the linker between the VL and VH domains is single underlined, the anti-CD7 VH domain is in bold/single underlined, CD8a hinge and transmembrane domain is double underlined, 4-1BB signaling domain is between the CD8a hinge and transmembrane domain and the CD3ζ signaling domain, CD3ζ signaling domain is bold/double underlined, and a stop codon ends the sequence.

FIG. 19 depicts the nucleic acid sequence of an exemplary anti-human CD7 CAR based on the antibody 3A1F (SEQ ID NO:5). The CD8 signal peptide starts at position 1, the anti-CD7 VL domain is in bold, the linker between the VL and VH domains is single underlined, the anti-CD7 VH domain is in bold/single underlined, CD8a hinge and transmembrane domain is double underlined, 4-1BB signaling domain is between the CD8a hinge and transmembrane domain and the CD3ζ signaling domain, CD3ζ signaling domain is bold/double underlined, and a stop codon ends the sequence.

FIG. 20 depicts the nucleic acid sequence of an exemplary CMV promoter (SEQ ID NO:6).

FIG. 21 depicts the nucleic acid sequence of an exemplary EF1α promoter (SEQ ID NO:7).

FIG. 22 depicts the nucleic acid sequence of an exemplary EFS promoter (SEQ ID NO:8).

FIG. 23 depicts the nucleic acid sequence of an exemplary MSCV promoter (SEQ ID NO:9).

FIG. 24 depicts the nucleic acid sequence of an exemplary PGK promoter (SEQ ID NO: 10).

FIG. 25 depicts the nucleic acid sequence of an exemplary bicistronic construct comprising anti-human CD7 (TH69) PEBL-IRES-anti-human CD7 (TH69) CAR (SEQ ID NO:11). Anti-human CD7 (TH69) PEBL is in normal font, IRES is bold, and anti-human CD7 (TH69) CAR is double underlined.

FIG. 26 depicts the nucleic acid sequence of an exemplary bicistronic construct comprising anti-human CD7 (TH69) CAR-IRES-anti-human CD7 (TH69) PEBL (SEQ ID NO:12). Anti-human CD7 (TH69) CAR is in normal font, IRES is in bold, and anti-human CD7 (TH69) PEBL is single underlined.

FIG. 27 depicts the nucleic acid sequence of an exemplary bicistronic construct comprising anti-human CD7 (TH69) CAR-P2A-anti-human CD7 (TH69) PEBL (SEQ ID NO:13). Anti-human CD7 (TH69) CAR is in normal font, P2A is in bold, and anti-human CD7 (TH69) PEBL is single underlined.

FIG. 28A and FIG. 28B depict the nucleic acid sequence of an exemplary bicistronic construct comprising an MSCV promoter-anti-human CD7 (TH69) CAR-P2A-anti-human CD7 (TH69) PEBL (SEQ ID NO:14). The MSCV promoter is double underlined, a restriction enzyme site and Kozak sequence are between the MSCV promoter and CAR, anti-human CD7 (TH69) CAR is in bold, P2A is in normal font, and anti-human CD7 (TH69) PEBL is single underlined.

FIG. 29A and FIG. 29B depict the nucleic acid sequence of an exemplary bicistronic construct comprising an EF1a promoter-anti-human CD7 (TH69) CAR-P2A-anti-human CD7 (TH69) PEBL (SEQ ID NO:15). The EF1α promoter is double underlined, a restriction enzyme site and Kozak sequence are between the EF1α promoter and CAR, anti-human CD7 (TH69) CAR is in bold, P2A is in normal font, and anti-human CD7 (TH69) PEBL is single underlined.

FIG. 30A and FIG. 30B depict the nucleic acid sequence of an exemplary bicistronic construct comprising an EFS promoter-anti-human CD7 (TH69) CAR-P2A-anti-human CD7 (TH69) PEBL (SEQ ID NO:16). The EFS promoter is double underlined, a restriction enzyme site and Kozak sequence are between the EFS promoter and CAR, anti-human CD7 (TH69) CAR is in bold, P2A is in normal font, and anti-human CD7 (TH69) PEBL is single underlined.

FIG. 31A and FIG. 31B depict the nucleic acid sequence of an exemplary dual promoter construct comprising a MSCV promoter-anti-human CD7 (TH69) CAR-PGK promoter-anti-human CD7 (TH69) PEBL (SEQ ID NO:17). The MSCV promoter is double underlined, a restriction enzyme site (in italics) and Kozak sequence are between the MSCV promoter and CAR, anti-human CD7 (TH69) CAR is in bold, a restriction enzyme site (in italics) is between CAR and PGK promoter, PGK promoter is single underlined, a restriction enzyme site (italized) and Kozak sequence are between PGK promoter and PEBL, anti-human CD7 (TH69) PEBL is bold/single underlined, and restriction enzyme site (in italics) ends the sequence.

FIG. 32A and FIG. 32B depict the nucleic acid sequence of an exemplary dual promoter construct comprising a MSCV promoter-anti-human CD7 (TH69) CAR-EF1a promoter-anti-human CD7 (TH69) PEBL (SEQ ID NO:18). The MSCV promoter is double underlined, a restriction enzyme site (in italics) and Kozak sequence are between the MSCV promoter and CAR, anti-human CD7 (TH69) CAR is in bold, a restriction enzyme site (in italics) is between CAR and EF1a promoter, EF1a promoter is single underlined, a restriction enzyme site (in italics) and Kozak sequence are between EF1a promoter and PEBL, and anti-human CD7 (TH69) PEBL is bold/single underlined.

FIG. 33A and FIG. 33B depict the nucleic acid sequence of an exemplary dual promoter construct comprising a MSCV promoter-anti-human CD7 (TH69) CAR-EFS promoter-anti-human CD7 (TH69) PEBL (SEQ ID NO:19). The MSCV promoter is double underlined, a restriction enzyme site (in italics) and Kozak sequence are between the MSCV promoter and CAR, anti-human CD7 (TH69) CAR is in bold, a restriction enzyme site (in italics) is between CAR and EFS promoter, EFS promoter is single underlined, a restriction enzyme site (in italics) and Kozak sequence are between EFS promoter and PEBL, and anti-human CD7 (TH69) PEBL is bold/single underlined.

FIG. 34A and FIG. 34B depict the nucleic acid sequence of an exemplary dual promoter construct comprising a PGK promoter-anti-human CD7 (3A1F) CAR-EF1a promoter-anti-human CD7 (TH69) PEBL (SEQ ID NO:20). The PGK promoter is double underlined, a restriction enzyme site (in italics) and Kozak sequence are between the PGK promoter and CAR, anti-human CD7 (3A1F) CAR is in bold, a restriction enzyme site (in italics) is between CAR and EF1a promoter, EF1a promoter is single underlined, a restriction enzyme site (in italics) and Kozak sequence are between EF1a promoter and PEBL, and anti-human CD7 (TH69) PEBL is bold/single underlined.

FIG. 35A and FIG. 35B depict the nucleic acid sequence of an exemplary dual promoter construct comprising a PGK promoter-anti-human CD7 (TH69) CAR-EF1a promoter-anti-human CD7 (3A1F) PEBL (SEQ ID NO:21). The PGK promoter is double underlined, a restriction enzyme site (in italics) and Kozak sequence are between the PGK promoter and CAR, anti-human CD7 (TH69) CAR is in bold, a restriction enzyme site (in italics) is between CAR and EF1a promoter, EF1a promoter is single underlined, a restriction enzyme site (in italics) and Kozak sequence are between EF1a promoter and PEBL, and anti-human CD7 (3A1F) PEBL is bold/single underlined.

FIG. 36A-FIG. 36C depict the nucleic acid sequence of an exemplary dual promoter construct comprising a PGK promoter-anti-human CD7 (TH69) CAR-EF1a promoter-anti-human CD7 (TH69) PEBL (SEQ ID NO:22). The PGK promoter is double underlined, a restriction enzyme site (in italics) and Kozak sequence are between the PGK promoter and CAR, anti-human CD7 (TH69) CAR is in bold, a restriction enzyme site (in italics) is between CAR and EF1a promoter, EF1a promoter is single underlined, a restriction enzyme site (in italics) and Kozak sequence are between EF1a promoter and PEBL, and anti-human CD7 (TH69) PEBL is bold/single underlined.

FIG. 37A and FIG. 37B depict the nucleic acid sequence of an exemplary dual promoter construct comprising a PGK promoter-anti-human CD7 (TH69) CAR-EFS promoter-anti-human CD7 (TH69) PEBL (SEQ ID NO:23). The PGK promoter is double underlined, a restriction enzyme site (in italics) and Kozak sequence are between the PGK promoter and CAR, anti-human CD7 (TH69) CAR is in bold, a restriction enzyme site (in italics) is between CAR and EFS promoter, EFS promoter is single underlined, a restriction enzyme site (in italics) and Kozak sequence are between EFS promoter and PEBL, and anti-human CD7 (TH69) PEBL is bold/single underlined.

FIG. 38 depicts the amino acid sequence of an exemplary anti-human CD7 PEBL based on the antibody TH69 (SEQ ID NO:24). The CD8 signal peptide starts at position 1 and is in normal type, the anti-CD7 VL domain is in bold, the linker between the VL and VH domains is single underlined, the anti-CD7 VH domain is in bold/single underlined, and the myc-KDEL peptide is double underlined.

FIG. 39 depicts the amino acid sequence of an exemplary anti-human CD7 PEBL variant based on the antibody TH69 (SEQ ID NO:25). The N-terminal proline is in italics, the CD8 signal peptide starts at position 2 and is in normal type, the anti-CD7 VL domain is in bold, the linker between the VL and VH domains is single underlined, the anti-CD7 VH domain is in bold/single underlined, and the myc-KDEL peptide is double underlined.

FIG. 40 depicts the amino acid sequence of an exemplary anti-human CD7 PEBL based on the antibody 3A1F (SEQ ID NO:26). The CD8 signal peptide starts at position 1 and is in normal type, the anti-CD7 VL domain is in bold, the linker between the VL and VH domains is single underlined, the anti-CD7 VH domain is in bold/single underlined, and the myc-KDEL peptide is double underlined.

FIG. 41 depicts the amino acid sequence of an exemplary anti-human CD7 PEBL variant based on the antibody 3A1F (SEQ ID NO:27). The N-terminal proline is in italics, the CD8 signal peptide starts at position 2 and is in normal type, the anti-CD7 VL domain is in bold, the linker between the VL and VH domains is single underlined, the anti-CD7 VH domain is in bold/single underlined, and the myc-KDEL peptide is double underlined.

FIG. 42 depicts the amino acid sequence of an exemplary anti-human CD7 CAR based on the antibody TH69 (SEQ ID NO:28). The CD8 signal peptide starts at position 1, the anti-CD7 VL domain is in bold, the linker between the VL and VH domains is single underlined, the anti-CD7 VH domain is in bold/single underlined, CD8a hinge and transmembrane domain is double underlined, 4-1BB signaling domain is between the CD8a hinge and transmembrane domain and the CD3ζ signaling domain and is in normal type, and CD3ζ signaling domain is bold/double underlined.

FIG. 43 depicts the amino acid sequence of an exemplary anti-human CD7 CAR variant based on the antibody TH69 (SEQ ID NO:29). The CD8 signal peptide starts at position 1, the anti-CD7 VL domain is in bold, the linker between the VL and VH domains is single underlined, the anti-CD7 VH domain is in bold/single underlined, CD8a hinge and transmembrane domain is double underlined, 4-1BB signaling domain is between the CD8a hinge and transmembrane domain and the CD3ζ signaling domain and is in normal type, CD3ζ signaling domain is bold/double underlined, and the amino acids at the C-terminus of the CD3ζ signaling domain arise via ribosome skipping at the P2A site.

FIG. 44 depicts the amino acid sequence of an exemplary anti-human CD7 CAR based on the antibody 3A1F (SEQ ID NO:30). The CD8 signal peptide starts at position 1, the anti-CD7 VL domain is in bold, the linker between the VL and VH domains is single underlined, the anti-CD7 VH domain is in bold/single underlined, CD8a hinge and transmembrane domain is double underlined, 4-1BB signaling domain is between the CD8a hinge and transmembrane domain and the CD3ζ signaling domain and is in normal type, and CD3ζ signaling domain is bold/double underlined.

FIG. 45 depicts the amino acid sequence of an exemplary anti-human CD7 CAR variant based on the antibody 3A1F (SEQ ID NO:31). The CD8 signal peptide starts at position 1, the anti-CD7 VL domain is in bold, the linker between the VL and VH domains is single underlined, the anti-CD7 VH domain is in bold/single underlined, CD8a hinge and transmembrane domain is double underlined, 4-1BB signaling domain is between the CD8a hinge and transmembrane domain and the CD3ζ signaling domain and is in normal type, CD3ζ signaling domain is bold/double underlined, and the amino acids at the C-terminus of the CD3ζ signaling domain arise via ribosome skipping at the P2A site.

FIG. 46 depicts the amino acid sequence of an exemplary anti-human CD7 CAR based on the antibody TH69-P2A-anti-human CD7 PEBL based on the antibody TH69 (SEQ ID NO:95). The CD7 CAR is in normal font, the P2A is double underlined, and the CD7 PEBL is bold/single underlined.

### DETAILED DESCRIPTION OF THE INVENTION

### Introduction

The present invention provides methods for simultaneous expression of a fratricide-inducing chimeric antigen receptor (e.g., CAR) and a fratricide-preventing protein (e.g., PEBL) in T cells that result in viable CAR-expressing cytotoxic T lymphocytes (CAR-T) that target T cell antigens.

Viral vectors have been produced in which two or more genes can be expressed from a single construct. Typically, these vectors employ either a bicistronic element or a two-promoter configuration. In the case of bicistronic vectors, a sequence element is introduced between two genes that enables the translation of two proteins from a single messenger RNA. Examples include the internal ribosome entry site sequences (IRES) and the virally derived “codon skipping” peptide sequences such as P2A, T2A, F2A, E2A, and the like. In the case of two-promoter designed vectors, separate promoter elements are configured upstream of each gene such that each promoter transcribes the mRNA for its proximally linked gene. In some embodiments, an expression vector (e.g., construct) contains a first promoter operably linked to a CAR and a second promoter operably linked to a PEBL.

Described herein are methods for producing and testing various bicistronic vectors and two-promoter designed vectors for the expression of two different genes (e.g., a gene encoding a CAR, and a gene encoding a PEBL). It was unexpectedly discovered that certain two gene vectors were able to direct expression of both a PEBL and a CAR protein in T cells in a manner such that the resulting engineered T cells survived, expanded, and were able to kill target cells. The relative timing and level of expression of each gene in the identified two gene vectors enabled the downregulation of the target antigen before the CAR can cause undue fratricide to the engineered T cells.

Described herein are fratricide-resistant CAR-T cells expressing a CAR directed against CD7 and such CAR-T cell has reduced or no surface expression of CD7. The present invention is based, in part, on co-expression of a chimeric antigen receptor (CAR) directed against CD7 and a protein expression blocker (PEBL) directed against CD7 in immune cells (e.g., T cells) using a bicistronic construct, such as a bicistronic viral vector. In one aspect, the present invention relates to an engineered immune cell (e.g., an engineered T cell) comprising a bicistronic construct comprising a polynucleotide sequence encoding an anti-CD7 CAR and a polynucleotide sequence encoding an anti-CD7 PEBL. In some embodiments, the CAR comprises intracellular signaling domains of 4-1BB and CD3ζ, and an antibody (e.g., a single chain variable fragment or scFv) that specifically binds CD7. The CD7 CAR of the present invention is sometimes referred to herein as “anti-CD7-41BB-CD3ζ”. In some embodiments, the CAR also includes a CD8a hinge and transmembrane domain. In some embodiments, the anti-CD7 PEBL comprises an antibody (e.g., a scFv) that specifically binds CD7 and an intracellular localization sequence. In certain embodiments, the anti-CD7 PEBL comprises an antibody (e.g., a scFv) that specifically binds CD7, CD8a hinge and transmembrane domains, and an intracellular localization sequence.

CD7 is a 40 kDa type I transmembrane glycoprotein which is the primary marker for T cell malignancies, and which is highly expressed in all cases of T cell ALL, including early T-cell progenitor acute lymphoblastic leukemia (ETP-ALL). An anti-CD7 CAR can induce T cells to exert specific cytotoxicity against T cell malignancies. Further, T cell cytotoxicity has been shown to be markedly increased when an anti-CD7 CAR was used in combination with downregulation of CD7 expression on the effector T cells. Downregulation (e.g., elimination, reduction, and/or relocalization) of CD7 in a T cell via expression of anti-CD7 PEBL prevented the fratricidal effect exerted by the corresponding anti-CD7 CAR. This led to greater T cell recovery after CAR expression as compared to cells that retained the target antigen (e.g., CD7), and a more effective cytotoxicity against T leukemia/lymphoma cells.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

As used herein, the term “nucleic acid” refers to a polymer comprising multiple nucleotide monomers (e.g., ribonucleotide monomers or deoxyribonucleotide monomers). “Nucleic acid” includes, for example, genomic DNA, cDNA, RNA, and DNA-RNA hybrid molecules. Nucleic acid molecules can be naturally occurring, recombinant, or synthetic. In addition, nucleic acid molecules can be single-stranded, double-stranded or triple-stranded. In certain embodiments, nucleic acid molecules can be modified. In the case of a double-stranded polymer, “nucleic acid” can refer to either or both strands of the molecule. Nucleic acids and polynucleotides as used herein are interchangeable.

The term “nucleotide sequence,” in reference to a nucleic acid, refers to a contiguous series of nucleotides that are joined by covalent linkages, such as phosphorus linkages (e.g., phosphodiester, alkyl and aryl-phosphonate, phosphorothioate, phosphotriester bonds), and/or non-phosphorus linkages (e.g., peptide and/or sulfamate bonds). In certain embodiments, the nucleotide sequence encoding, e.g., a target-binding molecule linked to a localizing domain is a heterologous sequence (e.g., a gene that is of a different species or cell type origin).

The terms “nucleotide” and “nucleotide monomer” refer to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., adenosine, thymidine, guanosine, cytidine, uridine, inosine, deoxyadenosine, deoxythymidine, deoxyguanosine, or deoxycytidine) and nucleotides comprising modified bases known in the art.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “sequence identity” means that two nucleotide sequences or two amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 95% sequence identity or more. For sequence comparison, typically one sequence acts as a reference sequence (e.g., parent sequence), to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

As will be appreciated by those of skill in the art, in some aspects, the nucleic acid further comprises a plasmid sequence. The plasmid sequence can include, for example, one or more sequences of a promoter sequence, a selection marker sequence, or a locus-targeting sequence.

The term “promoter” or “promoter element” as used herein is defined as a DNA sequence recognized by the synthetic machinery of the cell, or introduced synthetic machinery, required to initiate the specific transcription of a polynucleotide sequence.

The term retroviral vector” can refer to a gammaretroviral vector. A retroviral vector may include, e.g., a promoter, a packaging signal, a primer binding site (PBS), one or more (e.g., two) long terminal repeats (LTR), and polynucleotides of interest, e.g., a polynucleotide encoding a CAR and a polynucleotide encoding a PEBL. A retroviral vector may lack viral structural genes such as gag, pol, and env. Exemplary retroviral (e.g., gammaretroviral) vectors include Murine Embryonic Stem Cell Virus (MESV), Murine Stem Cell Virus (MSCV), Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Maetzig et al., Viruses, 2011; 3(6): 677-713.

The term “bicistronic expression” is typically achieved by operably linking the polynucleotides described herein to a promoter, and incorporating the bicistronic construct into an expression vector. The vectors can be suitable for replication and integration eukaryotes. Typical cloning vectors contain transcription and translation terminators, initiation sequences, and promoters useful for regulation of the expression of the desired nucleic acid sequence. The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the immediate early cytomegalovirus (CMV), EF-1α, ubiquitin C, or phosphoglycerokinase (PGK) promoters. A strong constitutive promoter sequence capable of driving high levels of expression of any polynucleotide sequence operatively linked thereto can be used. Other constitutive promoter sequences may be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1 Ovian leukemia virus promoter, an Epstein-Barr virus immediate early promoter, a Rous sarcoma virus promoter, as well as human gene promoters such as, but not limited to, the actin promoter, the myosin promoter, and the like. In some embodiments, the promoter is an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

As used herein, “antibody” means an intact antibody or antigen-binding fragment of an antibody, including an intact antibody or antigen-binding fragment modified or engineered, or that is a human antibody. Examples of antibodies modified or engineered are chimeric antibodies, humanized antibodies, multiparatopic antibodies (e.g., biparatopic antibodies), and multispecific antibodies (e.g., bispecific antibodies). Examples of antigen-binding fragments include Fab, Fab′, F(ab′)₂, Fv, single chain antibodies (e.g., scFv), minibodies and diabodies.

The term “specifically (or selectively) binds” or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

In certain embodiments, the antibody that binds CD7 is a single-chain variable fragment antibody (“scFv antibody”). scFv refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. For a review of scFv, see Pluckthun (1994) The Pharmacology Of Monoclonal Antibodies, vol. 113, Rosenburg and Moore eds. Springer-Verlag, New York, pp. 269-315. See also, PCT Publication No. WO 88/01649 and U.S. Pat. Nos. 4,946,778 and 5,260,203. As would be appreciated by those of skill in the art, various suitable linkers can be designed and tested for optimal function, as provided in the art, and as disclosed herein.

As used herein, an “engineered” immune cell includes an immune cell that has been genetically modified as compared to a naturally-occurring immune cell. For example, an engineered T cell produced according to the present methods carries a nucleic acid comprising a nucleotide sequence that does not naturally occur in a T cell from which it was derived, such as the nucleic acids exemplified herein.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

As used herein, a “CD7 CAR+/CD7-negative” T cell refers to a T cell expressing a chimeric antigen receptor against human CD7 and having low or no surface expression of endogenous CD7. In some embodiments, the low or no surface expression of endogenous CD7 is due to expression of a PEBL against human CD7 which prevents or hinders endogenous CD7 protein to translocated to the surface of the T cell. In some instances, surface expression of CD7 can be determined using standard methods known to those in the art such as but not limited to immunocytochemistry, flow cytometry, or FACS.

The term “autologous” and its grammatical equivalents as used herein can refer to as originating from the same being. For example, a sample (e.g., cells) can be removed, processed, and given back to the same subject (e.g., patient) at a later time. An autologous process is distinguished from an allogenic process where the donor and the recipient are different subjects.

“Allogeneic” refers to a graft derived from a different animal of the same species.

As used herein, the terms “treat,” “treating,” or “treatment,” refer to counteracting a medical condition (e.g., a condition related to a T cell malignancy) to the extent that the medical condition is improved according to a clinically-acceptable standard.

As used herein, “subject” refers to a mammal (e.g., human, non-human primate, cow, sheep, goat, horse, dog, cat, rabbit, guinea pig, rat, mouse). In certain embodiments, the subject is a human. A “subject in need thereof” refers to a subject (e.g., patient) who has, or is at risk for developing, a disease or condition that can be treated (e.g., improved, ameliorated, prevented) by inducing T cells to exert specific cytotoxicity against malignant T cells.

As defined herein, a “therapeutic amount” refers to an amount that, when administered to a subject, is sufficient to achieve a desired therapeutic effect (treats a condition related to a T cell malignancy) in the subject under the conditions of administration. An effective amount of the agent to be administered can be determined by a clinician of ordinary skill using the guidance provided herein and other methods known in the art, and is dependent on several factors including, for example, the particular agent chosen, the subject's age, sensitivity, tolerance to drugs and overall well-being.

### DETAILED DESCRIPTION OF THE EMBODIMENTS

As described in detail below, the anti-CD7 CAR (also referred to as “CD7 CAR”) can comprise an antigen binding domain targeting CD7 based on the TH69 antibody. In some embodiments, the antigen binding domain of the CD7 CAR is based on the 3A1F antibody. In some embodiments, the antigen binding domain of the CD7 CAR is based on the T3-3A1 antibody. In some embodiments, the CD7 CAR of the present invention comprises an amino acid sequence selected from the group consisting of SEQ ID NO:28, SEQ ID NO:29, SEQ ID NO:30, and SEQ ID NO:31. In some embodiments, the CD7 CAR comprises an amino acid sequence having at least 90% sequence identity to one selected from the group consisting of SEQ ID NO:28, SEQ ID NO:29, SEQ ID NO:30, and SEQ ID NO:31. In some cases, the engineered immune cell of the present invention comprises the CD7 CAR of SEQ ID NO:28. In some cases, the engineered immune cell comprises the CD7 CAR having at least 90% sequence identity to SEQ ID NO:28. In some cases, the engineered immune cell comprises the CD7 CAR of SEQ ID NO:29. In some cases, the engineered immune cell comprises the CD7 CAR having at least 90% sequence identity to SEQ ID NO:30. In some cases, the engineered immune cell comprises the CD7 CAR having at least 90% sequence identity to SEQ ID NO:30. In some cases, the engineered immune cell comprises the CD7 CAR having at least 90% sequence identity to SEQ ID NO:31. In some cases, the engineered immune cell comprises the CD7 CAR having at least 90% sequence identity to SEQ ID NO:31.

In some embodiments, the CD7 PEBL of the present invention comprises an amino acid sequence selected from the group consisting of SEQ ID NO:24, SEQ ID NO:25, SEQ ID NO:26, and SEQ ID NO:27. In some embodiments, the CD7 PEBL of the present invention comprises an amino acid sequence having at least 90% sequence identity to one selected from the group consisting of SEQ ID NO:24, SEQ ID NO:25, SEQ ID NO:26, and SEQ ID NO:27. In some instances, the engineered immune cell of the present invention comprises the CD7 PEBL of SEQ ID NO: 24. In some instances, the engineered immune cell of the present invention comprises the CD7 PEBL having at least 90% sequence identity to SEQ ID NO: 25. In some instances, the engineered immune cell comprises the CD7 PEBL of SEQ ID NO: 24. In some instances, the engineered immune cell comprises the CD7 PEBL having at least 90% sequence identity to SEQ ID NO: 25. In some instances, the engineered immune cell comprises the CD7 PEBL of SEQ ID NO: 26. In some instances, the engineered immune cell comprises the CD7 PEBL having at least 90% sequence identity to SEQ ID NO: 26. In some instances, the engineered immune cell comprises the CD7 PEBL of SEQ ID NO: 27. In some instances, the engineered immune cell comprises the CD7 PEBL having at least 90% sequence identity to SEQ ID NO: 27.

In some embodiments, the engineered immune cell or population of engineered immune cells of the present invention comprises a CD7 PEBL of SEQ ID NO: 24 and a CD7 CAR of SEQ ID NO:28. In some embodiments, the engineered immune cell or population of engineered immune cells comprises a CD7 PEBL having at least 90% sequence identity to SEQ ID NO: 24 and a CD7 CAR having at least 90% sequence identity to SEQ ID NO:28. In some embodiments, the engineered immune cell or population of engineered immune cells comprises a CD7 PEBL of SEQ ID NO: 24 and a CD7 CAR of SEQ ID NO:30. In some embodiments, the engineered immune cell or population of engineered immune cells comprises a CD7 PEBL having at least 90% sequence identity SEQ ID NO: 24 and a CD7 CAR having at least 90% sequence identity SEQ ID NO:30. In some embodiments, the engineered immune cell or population of engineered immune cells comprises a CD7 PEBL of SEQ ID NO: 26 and a CD7 CAR of SEQ ID NO:28. In some embodiments, the engineered immune cell or population of engineered immune cells comprises a CD7 PEBL having at least 90% sequence identity SEQ ID NO: 26 and a CD7 CAR having at least 90% sequence identity SEQ ID NO:28. In some embodiments, the engineered immune cell or population of engineered immune cells comprises a CD7 PEBL of SEQ ID NO: 26 and a CD7 CAR of SEQ ID NO:308. In some embodiments, the engineered immune cell or population of engineered immune cells comprises a CD7 PEBL having at least 90% sequence identity SEQ ID NO: 26 and a CD7 CAR having at least 90% sequence identity SEQ ID NO:30. In some embodiments, the engineered immune cell or population of engineered immune cells comprises a CD7 PEBL of SEQ ID NO: 25 and a CD7 CAR of SEQ ID NO:29. In some embodiments, the engineered immune cell or population of engineered immune cells comprises a CD7 PEBL having at least 90% sequence identity SEQ ID NO: 25 and a CD7 CAR having at least 90% sequence identity SEQ ID NO:29. In some embodiments, the engineered immune cell or population of engineered immune cells comprises a CD7 PEBL of SEQ ID NO: 25 and a CD7 CAR of SEQ ID NO:31. In some embodiments, the engineered immune cell or population of engineered immune cells comprises a CD7 PEBL having at least 90% sequence identity SEQ ID NO: 25 and a CD7 CAR having at least 90% sequence identity SEQ ID NO:31. In some embodiments, the engineered immune cell or population of engineered immune cells comprises a CD7 PEBL of SEQ ID NO: 27 and a CD7 CAR of SEQ ID NO:29. In some embodiments, the engineered immune cell or population of engineered immune cells comprises a CD7 PEBL having at least 90% sequence identity SEQ ID NO: 27 and a CD7 CAR having at least 90% sequence identity SEQ ID NO:29. In some embodiments, the engineered immune cell or population of engineered immune cells comprises a CD7 PEBL of SEQ ID NO: 27 and a CD7 CAR of SEQ ID NO:31. In some embodiments, the engineered immune cell or population of engineered immune cells comprises a CD7 PEBL having at least 90% sequence identity SEQ ID NO: 27 and a CD7 CAR having at least 90% sequence identity SEQ ID NO:31.

In certain embodiments, the engineered immune cell is an engineered T cell. In some embodiments, the engineered immune cell is an engineered CD4+ T cell. In some embodiments, the engineered immune cell is an engineered CD8+ T cell. In some embodiments, the engineered immune cell harboring the bicistronic construct or dual-promoter construct is generated from PBMCs. In some embodiments, the engineered immune cell harboring the bicistronic construct or dual-promoter construct is generated from purified CD4+ T cells. In some embodiments, the engineered immune cell harboring the bicistronic construct or dual-promoter construct is generated from purified CD8+ T cells. In some embodiments, the engineered immune cell harboring the bicistronic construct or dual-promoter construct is generated from a population for cells comprising purified CD4+ T cells and purified CD8+ T cells. In some embodiments, the engineered immune cell harboring the bicistronic construct or dual-promoter construct is generated from a population for cells comprising purified CD3+ T cells.

### Bicistronic Expression Constructs

Provided herein are recombinant bicistronic viral constructs or vectors that contain a polynucleotide encoding a CAR and a polynucleotide encoding a PEBL, as described herein. In some embodiments, the recombinant bicistronic viral construct includes an internal ribosomal entry site (IRES) sequence between the nucleic acid sequence of the CAR and the nucleic acid sequence of the PEBL. In some embodiments, the recombinant bicistronic viral construct includes a ribosomal codon skipping site sequence (also referred to as a sequence encoding a 2A self-cleaving peptide) between the nucleic acid sequence of the CAR and the nucleic acid sequence of the PEBL. In some embodiments of a bicistronic construct, a polynucleotide encoding a CAR is located upstream (at the 5′ end) of an IRES sequence, and a polynucleotide encoding a PEBL is located downstream (at the 3′ end) of the IRES. In some cases, a nucleic acid sequence encoding a CAR is operably linked to an IRES sequence and an IRES sequence is operably linked to a nucleic acid sequence encoding a PEBL. In some cases, a nucleic acid sequence encoding a PEBL is operably linked to an IRES sequence and an IRES sequence is operably linked to a nucleic acid sequence encoding a CAR.

In some embodiments of a bicistronic construct, a polynucleotide encoding a CAR is located upstream (at the 5′ end) of a polynucleotide encoding 2A self-cleaving peptide, and a polynucleotide encoding a PEBL is located downstream (at the 3′ end) of the polynucleotide encoding 2A self-cleaving peptide. In some cases, a nucleic acid sequence encoding a CAR is operably linked to a nucleic acid sequence encoding a 2A self-cleaving peptide, which is operably linked to a nucleic acid sequence encoding a PEBL. In some cases, a nucleic acid sequence encoding a PEBL is operably linked to a nucleic acid sequence encoding a 2A self-cleaving peptide, which is operably linked to a nucleic acid sequence encoding a CAR.

The mechanism of ribosomal codon skipping via a 2A peptide sequence is useful for generating two proteins from one transcript; a normal peptide bond is impaired at the 2A sequence, resulting in two discontinuous protein fragments from one translation event. Self-cleaving 2A peptides (e.g., 2A cleavage sites) are described in Kim et al., PLoS One, 2011, 6(4):e18556.

In some embodiments, the IRES is from an Encephalomyocarditis virus. In some embodiments, the IRES is from an Enterovirus. In some embodiments, the nucleic acid sequence of the IRES sequence is set forth in SEQ ID NO:62 (see, e.g., Table 1).

In some embodiments, the ribosomal codon skipping site is based on a 2A self-cleaving peptide (see, e.g., Table 2). In some embodiments, the 2A self-cleaving peptide is selected from the group consisting of P2A, E2A, F2A, and T2A. In some instances, the amino acid sequence of the P2A peptide comprises the amino acid sequence of SEQ ID NO:67, or an amino acid sequence having at least 90% sequence identify thereto. In some instances, the amino acid sequence of the E2A peptide comprises the amino acid sequence of SEQ ID NO:68, or an amino acid sequence having at least 90% sequence identify thereto. In some instances, the amino acid sequence of the F2A peptide comprises the amino acid sequence of SEQ ID NO:69, or an amino acid sequence having at least 90% sequence identify thereto. In some instances, the amino acid sequence of the T2A peptide comprises the amino acid sequence of SEQ ID NO:70, or an amino acid sequence having at least 90% sequence identify thereto.

In some embodiments, the viral construct (e.g., retroviral construct) comprises a nucleic acid sequence encoding a 2A self-cleaving peptide (e.g., 2A peptide cleavage site) selected from the group consisting of P2A, E2A, F2A, and T2A, wherein the polynucleotide encoding 2A self-cleaving peptide links the nucleic acid sequence encoding the CAR and the nucleic acid sequence encoding the PEBL. In other words, the polynucleotide encoding 2A self-cleaving peptide is between the nucleic acid sequence encoding the CAR and the nucleic acid sequence encoding the PEBL. As described above, in some embodiments, the construct comprises or consisting of from 5′ end to 3′ end: a nucleic acid sequence encoding a CAR, a nucleic acid sequence encoding a P2A self-cleaving peptide, and a nucleic acid sequence encoding a PEBL. In some embodiments, the construct comprises or consisting of from 5′ end to 3′ end: a nucleic acid sequence encoding any CD7 CAR described herein, a nucleic acid sequence encoding a P2A self-cleaving peptide, and a nucleic acid sequence encoding any CD7 PEBL described herein. In some embodiments, the construct comprises or consisting of from 5′ end to 3′ end: a nucleic acid sequence encoding any CD7 CAR described herein, a nucleic acid sequence encoding an E2A self-cleaving peptide, and a nucleic acid sequence encoding any CD7 PEBL described herein. In some embodiments, the construct comprises or consisting of from 5′ end to 3′ end: a nucleic acid sequence encoding any CD7 CAR described herein, a nucleic acid sequence encoding an F2A self-cleaving peptide, and a nucleic acid sequence encoding any CD7 PEBL described herein. In some embodiments, the construct comprises or consisting of from 5′ end to 3′ end: a nucleic acid sequence encoding any CD7 CAR described herein, a nucleic acid sequence encoding a T2A self-cleaving peptide, and a nucleic acid sequence encoding any CD7 PEBL described herein.

In some embodiments, the construct comprises or consisting of from 5′ end to 3′ end: a nucleic acid sequence encoding a PEBL, a nucleic acid sequence encoding a P2A self-cleaving peptide, and a nucleic acid sequence encoding a CAR. In some embodiments, the construct comprises or consisting of from 5′ end to 3′ end: a nucleic acid sequence encoding a PEBL, a nucleic acid sequence encoding an E2A self-cleaving peptide, and a nucleic acid sequence encoding a CAR. In some embodiments, the construct comprises or consisting of from 5′ end to 3′ end: a nucleic acid sequence encoding a PEBL, a nucleic acid sequence encoding an F2A self-cleaving peptide, and a nucleic acid sequence encoding a CAR. In some embodiments, the construct comprises or consisting of from 5′ end to 3′ end: a nucleic acid sequence encoding a PEBL, a nucleic acid sequence encoding a T2A self-cleaving peptide, and a nucleic acid sequence encoding a CAR.

In some embodiments, the nucleic acid sequence encoding the P2A comprises or consisting of a nucleic acid having at least 90% sequence identity to SEQ ID NO:63. In some embodiments, the nucleic acid sequence encoding the P2A comprises or consisting of a nucleic acid of SEQ ID NO:63. In some embodiments, the nucleic acid sequence encoding the E2A comprises or consisting of a nucleic acid having at least 90% sequence identity to SEQ ID NO:64. In some embodiments, the nucleic acid sequence encoding the E2A comprises or consisting of a nucleic acid of SEQ ID NO:64. In some embodiments, the nucleic acid sequence encoding the F2A comprises or consisting of a nucleic acid having at least 90% sequence identity to SEQ ID NO:65. In some embodiments, the nucleic acid sequence encoding the F2A comprises or consisting of a nucleic acid of SEQ ID NO:65. In some embodiments, the nucleic acid sequence encoding the T2A comprises or consisting of a nucleic acid sequence having at least 90% sequence identity to SEQ ID NO:66. In some embodiments, the nucleic acid sequence encoding the T2A comprises or consisting of a nucleic acid of SEQ ID NO:66.

In some embodiments, the nucleic acid sequence encoding the PEBL is disposed (e.g., located) 5′ to the nucleic acid sequence encoding the CAR. In some embodiments, the nucleic acid sequence encoding the CAR is disposed 5′ to the nucleic acid sequence encoding the PEBL. In some embodiments, a bicistronic construct comprises or consists of from 5′ to 3′ end: (a1) SEQ ID NO:4, SEQ ID NO:63, and SEQ ID NO:2; (a2) SEQ ID NO:4, SEQ ID NO:63, and SEQ ID NO:3; (a3) SEQ ID NO:5, SEQ ID NO:63, and SEQ ID NO:2; (a4) SEQ ID NO:5, SEQ ID NO:63, and SEQ ID NO:3; (b1) SEQ ID NO:4, SEQ ID NO:64, and SEQ ID NO: 2; (b2) SEQ ID NO:4, SEQ ID NO: 64, and SEQ ID NO:3; (b3) SEQ ID NO:5, SEQ ID NO: 64, and SEQ ID NO:2; (b4) SEQ ID NO:5, SEQ ID NO: 64, and SEQ ID NO:3; (c1) SEQ ID NO:4, SEQ ID NO:65, and SEQ ID NO:2; (c2) SEQ ID NO:4, SEQ ID NO: 65, and SEQ ID NO:3; (c3) SEQ ID NO:5, SEQ ID NO: 65, and SEQ ID NO:2; (c4) SEQ ID NO:5, SEQ ID NO: 65, and SEQ ID NO:3; (d1) SEQ ID NO:4, SEQ ID NO:66, and SEQ ID NO:2; (d2) SEQ ID NO:4, SEQ ID NO: 66, and SEQ ID NO:3; (d3) SEQ ID NO:5, SEQ ID NO: 66, and SEQ ID NO:2; (d4) SEQ ID NO:5, SEQ ID NO: 66, and SEQ ID NO:3; (e1) SEQ ID NO:2, SEQ ID NO:63, and SEQ ID NO:4; (e2) SEQ ID NO:3, SEQ ID NO:63, and SEQ ID NO:4; (e3) SEQ ID NO:2, SEQ ID NO:63, and SEQ ID NO:5; (e4) SEQ ID NO:3, SEQ ID NO:63, and SEQ ID NO:5; (f1) SEQ ID NO:2, SEQ ID NO:64, and SEQ ID NO:4; (f2) SEQ ID NO:3, SEQ ID NO:64, and SEQ ID NO:4; (f3) SEQ ID NO:2, SEQ ID NO:63, and SEQ ID NO:5; (f4) SEQ ID NO:3, SEQ ID NO:64, and SEQ ID NO:5; (g1) SEQ ID NO:2, SEQ ID NO:65, and SEQ ID NO:4; (g2) SEQ ID NO:3, SEQ ID NO:65, and SEQ ID NO:4; (g3) SEQ ID NO:2, SEQ ID NO:65, and SEQ ID NO:5; (g4) SEQ ID NO:3, SEQ ID NO:65, and SEQ ID NO:5; (h1) SEQ ID NO:2, SEQ ID NO:66, and SEQ ID NO:4; (h2) SEQ ID NO:3, SEQ ID NO:66, and SEQ ID NO:4; (h3) SEQ ID NO:2, SEQ ID NO:66, and SEQ ID NO:5; or (h4) SEQ ID NO:3, SEQ ID NO:66, and SEQ ID NO:5.

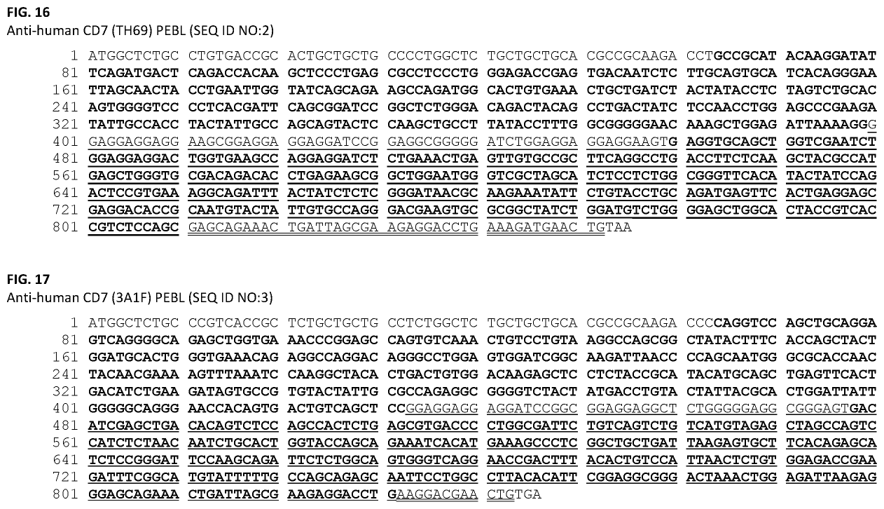

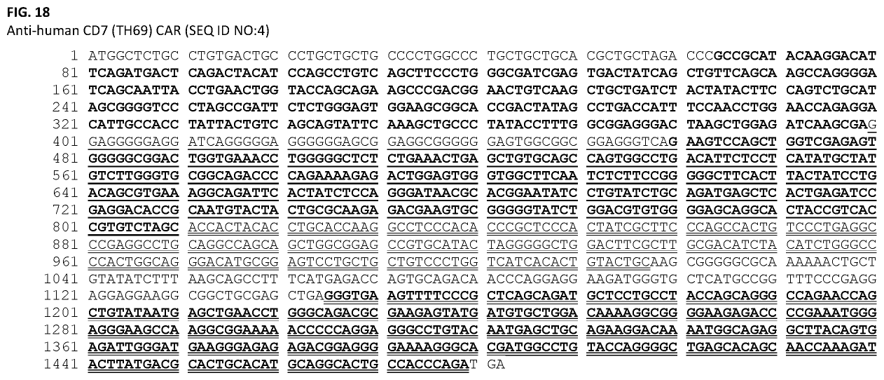

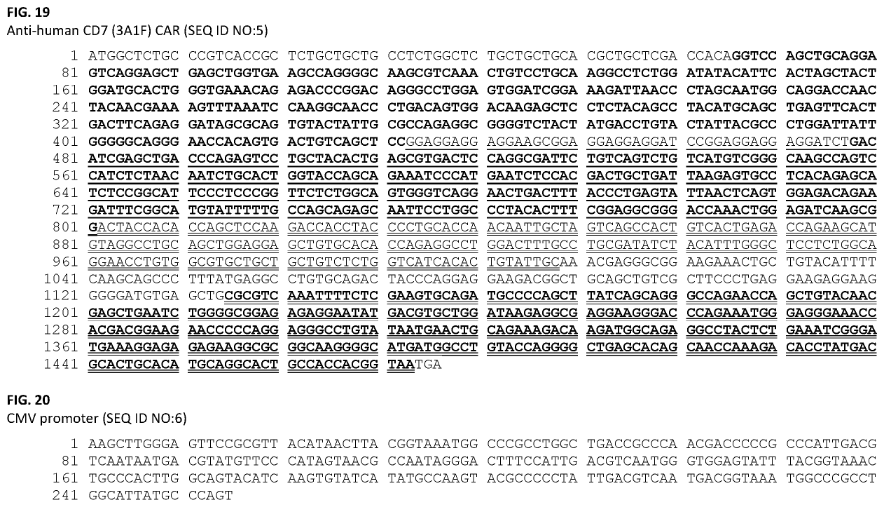

In some embodiments, a bicistronic construct comprises or consists of from 5′ to 3′ end: the sequence of FIG. 16, SEQ ID NO: 63, and the sequence of FIG. 18. In some embodiments, a bicistronic construct comprises or consists of from 5′ to 3′ end: the sequence of FIG. 16, any one of SEQ ID NOS: 63-66, and the sequence of FIG. 19. In some embodiments, a bicistronic construct comprises or consists of from 5′ to 3′ end: the sequence of FIG. 17, any one of SEQ ID NOS: 63-66, and the sequence of FIG. 18. In some embodiments, a bicistronic construct comprises or consists of from 5′ to 3′ end: the sequence of FIG. 17, any one of SEQ ID NOS: 63-66, and the sequence of FIG. 19.

In some embodiments, a bicistronic construct comprises or consists of from 5′ to 3′ end: the sequence of FIG. 18, any one of SEQ ID NOS: 63-66, and the sequence of FIG. 16. In some embodiments, a bicistronic construct comprises or consists of from 5′ to 3′ end: the sequence of FIG. 19, any one of SEQ ID NOS: 63-66, and the sequence of FIG. 16. In some embodiments, a bicistronic construct comprises or consists of from 5′ to 3′ end: the sequence of FIG. 18, any one of SEQ ID NOS: 63-66, and the sequence of FIG. 17. In some embodiments, a bicistronic construct comprises or consists of from 5′ to 3′ end: the sequence of FIG. 19, any one of SEQ ID NOS: 63-66, and the sequence of FIG. 17.

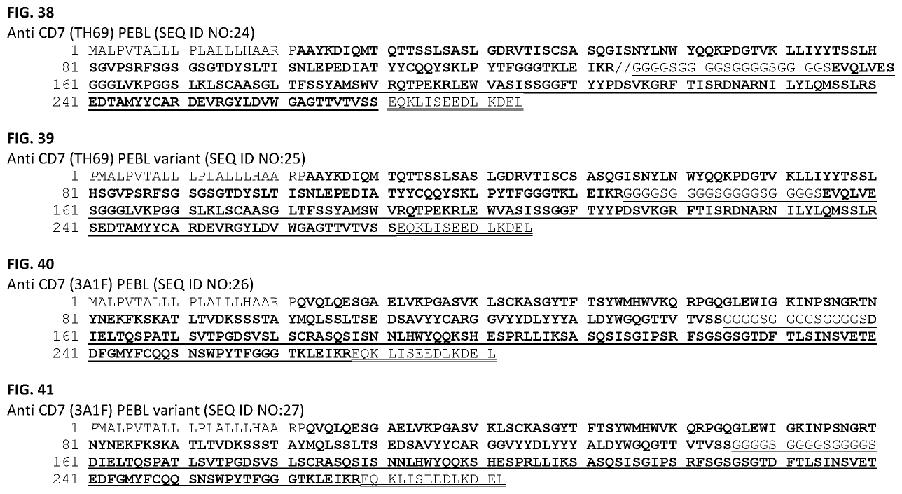

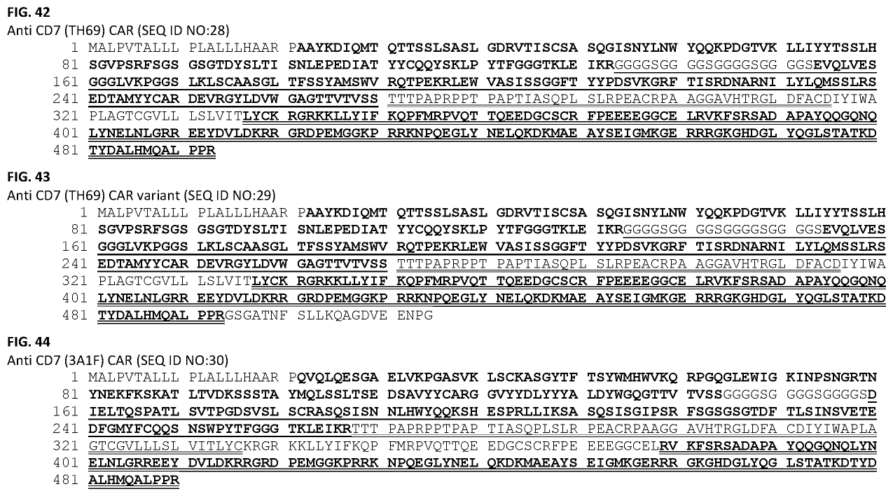

In some embodiments, a bicistronic construct comprises or consists of from 5′ to 3′ end: a polynucleotide encoding a CD7 (TH67) CAR of SEQ ID NO:28, a polynucleotide encoding a P2A peptide of SEQ ID NO:67, and a polynucleotide encoding a CD7 (TH67) PEBL of SEQ ID NO:24. In some embodiments, a bicistronic construct comprises or consists of from 5′ to 3′ end: a polynucleotide encoding a CD7 (TH67) CAR of FIG. 42, a polynucleotide encoding a P2A peptide of SEQ ID NO:67, and a polynucleotide encoding a CD7 (TH67) PEBL of FIG. 38. In some embodiments, a bicistronic construct comprises or consists of from 5′ to 3′ end: a polynucleotide encoding a CD7 (TH67) CAR of SEQ ID NO:28, a polynucleotide encoding a P2A peptide of SEQ ID NO:67, and a polynucleotide encoding a CD7 (3A1F) PEBL of SEQ ID NO:26. In some embodiments, a bicistronic construct comprises or consists of from 5′ to 3′ end: a polynucleotide encoding a CD7 (TH67) CAR of FIG. 42, a polynucleotide encoding a P2A peptide of SEQ ID NO:67, and a polynucleotide encoding a CD7 (3A1F) PEBL of FIG. 40. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:67, and a polynucleotide encoding a CD7 (TH67) PEBL of SEQ ID NO:24. In some embodiments, a bicistronic construct comprises or consists of from 5′ to 3′ end: a polynucleotide encoding a CD7 (3A1F) CAR of FIG. 44, a polynucleotide encoding a P2A peptide of SEQ ID NO:67, and a polynucleotide encoding a CD7 (TH67) PEBL of FIG. 38. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:67, and a polynucleotide encoding a CD7 (3A1F) PEBL of SEQ ID NO:26. In some embodiments, a bicistronic construct comprises or consists of from 5′ to 3′ end: a polynucleotide encoding a CD7 (3A1F) CAR of FIG. 44, a polynucleotide encoding a P2A peptide of SEQ ID NO:67, and a polynucleotide encoding a CD7 (3A1F) PEBL of FIG. 40. In some embodiments, a bicistronic construct comprises or consists of from 5′ to 3′ end: a polynucleotide encoding a CD7 (TH67) CAR of SEQ ID NO:28, a polynucleotide encoding a P2A peptide of SEQ ID NO:68, and a polynucleotide encoding a CD7 (TH67) PEBL of SEQ ID NO:24. In some embodiments, a bicistronic construct comprises or consists of from 5′ to 3′ end: a polynucleotide encoding a CD7 (TH67) CAR of FIG. 42, a polynucleotide encoding a P2A peptide of SEQ ID NO:68, and a polynucleotide encoding a CD7 (TH67) PEBL of FIG. 38. In some embodiments, a bicistronic construct comprises or consists of from 5′ to 3′ end: a polynucleotide encoding a CD7 (TH67) CAR of SEQ ID NO:28, a polynucleotide encoding a P2A peptide of SEQ ID NO:68, and a polynucleotide encoding a CD7 (3A1F) PEBL of SEQ ID NO:26. In some embodiments, a bicistronic construct comprises or consists of from 5′ to 3′ end: a polynucleotide encoding a CD7 (TH67) CAR of FIG. 42, a polynucleotide encoding a P2A peptide of SEQ ID NO:68, and a polynucleotide encoding a CD7 (3A1F) PEBL of FIG. 40. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:68, and a polynucleotide encoding a CD7 (TH67) PEBL of SEQ ID NO:24. In some embodiments, a bicistronic construct comprises or consists of from 5′ to 3′ end: a polynucleotide encoding a CD7 (3A1F) CAR of FIG. 44, a polynucleotide encoding a P2A peptide of SEQ ID NO:68, and a polynucleotide encoding a CD7 (TH67) PEBL of FIG. 38. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:68, and a polynucleotide encoding a CD7 (3A1F) PEBL of SEQ ID NO:26. In some embodiments, a bicistronic construct comprises or consists of from 5′ to 3′ end: a polynucleotide encoding a CD7 (3A1F) CAR of FIG. 44, a polynucleotide encoding a P2A peptide of SEQ ID NO:68, and a polynucleotide encoding a CD7 (3A1F) PEBL of FIG. 40. In some embodiments, a bicistronic construct comprises or consists of from 5′ to 3′ end: a polynucleotide encoding a CD7 (TH67) CAR of SEQ ID NO:28, a polynucleotide encoding a P2A peptide of SEQ ID NO:69, and a polynucleotide encoding a CD7 (TH67) PEBL of SEQ ID NO:24. In some embodiments, a bicistronic construct comprises or consists of from 5′ to 3′ end: a polynucleotide encoding a CD7 (TH67) CAR of FIG. 42, a polynucleotide encoding a P2A peptide of SEQ ID NO:69, and a polynucleotide encoding a CD7 (TH67) PEBL of FIG. 38. In some embodiments, a bicistronic construct comprises or consists of from 5′ to 3′ end: a polynucleotide encoding a CD7 (TH67) CAR of SEQ ID NO:28, a polynucleotide encoding a P2A peptide of SEQ ID NO:69, and a polynucleotide encoding a CD7 (3A1F) PEBL of SEQ ID NO:26. In some embodiments, a bicistronic construct comprises or consists of from 5′ to 3′ end: a polynucleotide encoding a CD7 (TH67) CAR of FIG. 42, a polynucleotide encoding a P2A peptide of SEQ ID NO:69, and a polynucleotide encoding a CD7 (3A1F) PEBL of FIG. 40. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:69, and a polynucleotide encoding a CD7 (TH67) PEBL of SEQ ID NO:24. In some embodiments, a bicistronic construct comprises or consists of from 5′ to 3′ end: a polynucleotide encoding a CD7 (3A1F) CAR of FIG. 44, a polynucleotide encoding a P2A peptide of SEQ ID NO:69, and a polynucleotide encoding a CD7 (TH67) PEBL of FIG. 38. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:69, and a polynucleotide encoding a CD7 (3A1F) PEBL of SEQ ID NO:26. In some embodiments, a bicistronic construct comprises or consists of from 5′ to 3′ end: a polynucleotide encoding a CD7 (3A1F) CAR of FIG. 44, a polynucleotide encoding a P2A peptide of SEQ ID NO:69, and a polynucleotide encoding a CD7 (3A1F) PEBL of FIG. 40. In some embodiments, a bicistronic construct comprises or consists of from 5′ to 3′ end: a polynucleotide encoding a CD7 (TH67) CAR of SEQ ID NO:28, a polynucleotide encoding a P2A peptide of SEQ ID NO:70, and a polynucleotide encoding a CD7 (TH67) PEBL of SEQ ID NO:24. In some embodiments, a bicistronic construct comprises or consists of from 5′ to 3′ end: a polynucleotide encoding a CD7 (TH67) CAR of FIG. 42, a polynucleotide encoding a P2A peptide of SEQ ID NO:70, and a polynucleotide encoding a CD7 (TH67) PEBL of FIG. 38. In some embodiments, a bicistronic construct comprises or consists of from 5′ to 3′ end: a polynucleotide encoding a CD7 (TH67) CAR of SEQ ID NO:28, a polynucleotide encoding a P2A peptide of SEQ ID NO:70, and a polynucleotide encoding a CD7 (3A1F) PEBL of SEQ ID NO:26. In some embodiments, a bicistronic construct comprises or consists of from 5′ to 3′ end: a polynucleotide encoding a CD7 (TH67) CAR of FIG. 42, a polynucleotide encoding a P2A peptide of SEQ ID NO:70, and a polynucleotide encoding a CD7 (3A1F) PEBL of FIG. 40. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:70, and a polynucleotide encoding a CD7 (TH67) PEBL of SEQ ID NO:24. In some embodiments, a bicistronic construct comprises or consists of from 5′ to 3′ end: a polynucleotide encoding a CD7 (3A1F) CAR of FIG. 44, a polynucleotide encoding a P2A peptide of SEQ ID NO:70, and a polynucleotide encoding a CD7 (TH67) PEBL of FIG. 38. In some embodiments, a bicistronic construct comprises or consists of from 5′ to 3′ end: a polynucleotide encoding a CD7 (3A1F) CAR of SEQ ID NO:30, a polynucleotide encoding a P2A peptide of SEQ ID NO:70, and a polynucleotide encoding a CD7 (3A1F) PEBL of SEQ ID NO:26. In some embodiments, a bicistronic construct comprises or consists of from 5′ to 3′ end: a polynucleotide encoding a CD7 (3A1F) CAR of FIG. 44, a polynucleotide encoding a P2A peptide of SEQ ID NO:70, and a polynucleotide encoding a CD7 (3A1F) PEBL of FIG. 40.

In some embodiments, the polynucleotide sequence encoding the PEBL is disposed 5′ (upstream) of an IRES site and the IRES site is disposed 5′ to the polynucleotide sequence encoding the CAR. In some embodiments, the polynucleotide sequence encoding the CAR is disposed 5′ of an IRES site and the IRES site is disposed 5′ to the polynucleotide sequence encoding the PEBL.

In some embodiments, the polynucleotide sequence encoding the PEBL is disposed 5′ (upstream) of the ribosomal codon skipping site and the ribosomal codon skipping site is disposed 5′ to the polynucleotide sequence encoding the CAR. In some embodiments, the polynucleotide sequence encoding the CAR is disposed 5′ of the ribosomal codon skipping site and the ribosomal codon skipping site is disposed 5′ to the polynucleotide sequence encoding the PEBL.

In some aspects, provided herein is a recombinant bicistronic construct comprising at least 90% sequence identity to a nucleic acid sequence of one or more selected from the group consisting of SEQ ID NO:63, SEQ ID NO:64, SEQ ID NO:65, and SEQ ID NO:66. In some embodiments, the recombinant bicistronic construct comprises at least 90% sequence identity to the nucleic acid sequence of SEQ ID NO:63. In some embodiments, the recombinant bicistronic construct comprises at least 90% sequence identity to the nucleic acid sequence of SEQ ID NO:64. In some embodiments, the recombinant bicistronic construct comprises at least 90% sequence identity to the nucleic acid sequence of SEQ ID NO:65. In some embodiments, the recombinant bicistronic construct comprises at least 90% sequence identity to the nucleic acid sequence of SEQ ID NO:66. In some embodiments, the recombinant bicistronic construct comprises an nucleic acid sequence of one selected from the group consisting of SEQ ID NO:63, SEQ ID NO:64, SEQ ID NO:65, and SEQ ID NO:66.

TABLE 1
Nucleic acid sequences of ribosomal codon
skipping peptides and IRES

| | SEQ ID | |
|---|---|---|
| Name | NO | Nucleic Acid Sequence |
| IRES | SEQ ID | CGGGATCAATTCCGCCCCCCCCCTAACGTTACTG |
| | NO: 62 | GCCGAAGCCGCTTGGAATAAGGCCGGTGTGCGTT |
| | | TGTCTATATGTTATTTTCCACCATATTGCCGTCT |
| | | TTTGGCAATGTGAGGGCCCGGAAACCTGGCCCTG |
| | | TCTTCTTGACGAGCATTCCTAGGGGTCTTTCCCC |
| | | TCTCGCCAAAGGAATGCAAGGTCTGTTGAATGTC |
| | | GTGAAGGAAGCAGTTCCTCTGGAAGCTTCTTGAA |
| | | GACAAACAACGTCTGTAGCGACCCTTTGCAGGCA |
| | | GCGGAACCCCCCACCTGGCGACAGGTGCCTCTGC |
| | | GGCCAAAAGCCACGTGTATAAGATACACCTGCAA |
| | | AGGCGGCACAACCCCAGTGCCACGTTGTGAGTTG |
| | | GATAGTTGTGGAAAGAGTCAAATGGCTCTCCTCA |
| | | AGCGTATTCAACAAGGGGCTGAAGGATGCCCAGA |
| | | AGGTACCCCATTGTATGGGATCTGATCTGGGGCC |
| | | TCGGTGCACATGCTTTACATGTGTTTAGTCGAGG |
| | | TTAAAAAAACGTCTAGGCCCCCCGAACCACGGGG |
| | | ACGTGGTTTTCCTTTGAAAAACACGATAATACC |
| P2A | SEQ ID | GCCACAAACTTCTCTCTGCTAAAGCAAGCAGGTG |
| | NO: 63 | ATGTTGAAGAAAACCCCGGGCCT |
| E2A | SEQ ID | CAGTGTACTAATTATGCTCTCTTGAAATTGGCTG |
| | NO: 64 | GAGATGTTGAGAGCAACGGAGGTCCC |
| F2A | SEQ ID | GTGAAACAGACTTTGAATTTTGACCTTCTCAAGT |
| | NO: 65 | TGGCGGGAGACGTGGAGTCCAACCCTGGACCT |
| T2A | SEQ ID | GAGGGCAGGGGAAGTCTTCTAACATGCGGGGACG |
| | NO: 66 | TGGAGGAAAATCCCGGCCCA |

TABLE 2
Amino acid sequences of ribosomal codon
skipping sites

| Name | SEQ ID NO | Amino Acid Sequence |
|---|---|---|
| P2A | SEQ ID NO: 67 | ATNFSLLKQAGDVEENPGP |
| E2A | SEQ ID NO: 68 | QCTNYALLKLAGDVESNPGP |
| F2A | SEQ ID NO: 69 | VKQTLNFDLLKLAGDVESNPGP |
| T2A | SEQ ID NO: 70 | EGRGSLLTCGDVEENPGP |

The present invention provides vectors such as expression vectors in which any of the polynucleotides described herein is inserted. In some embodiments, the vector is derived from retroviruses such as lentiviruses. Such vectors are suitable tools to achieve long-term gene transfer since they allow long-term, stable integration of an exogenous polynucleotide (e.g., transgene) and its propagation in daughter cells. Unlike vectors derived from onco-retroviruses such as murine leukemia viruses, lentiviral vectors can transduce non-proliferating cells. Lentiviral vectors also have low immunogenicity. In other embodiments, the vector is an adenoviral vector. In certain embodiments, the vector is a plasmid.

In some embodiments, the promoter comprises at least 90%, e.g., 90%, 91%, 92%, 93%, 94%, 95%, 96%, 97%, 98%, 99%, 99.9%, or more sequence identity to a CMV promoter. In some embodiments, the promoter comprises a CMV promoter. In some embodiments, the CMV promoter comprises the sequence of SEQ ID NO:6. In some embodiments, any of the constructs described herein comprises or consists of a CMV promoter of SEQ ID NO:6.

In some embodiments, the promoter comprises at least 90%, e.g., 90%, 91%, 92%, 93%, 94%, 95%, 96%, 97%, 98%, 99%, 99.9%, or more sequence identity to an EF1α promoter. In some embodiments, the promoter comprises an EF1α promoter. In some embodiments, the EF1a promoter comprises the sequence of SEQ ID NO:7. In some embodiments, the CMV promoter comprises the sequence of SEQ ID NO:6. In some embodiments, any of the constructs described herein comprises or consists of an EF1α promoter of SEQ ID NO:7.

In some embodiments, the promoter comprises at least 90%, e.g., 90%, 91%, 92%, 93%, 94%, 95%, 96%, 97%, 98%, 99%, 99.9%, or more sequence identity to an EFS promoter. In some embodiments, the promoter comprises an EFS promoter. In some embodiments, the EFS promoter comprises the sequence of SEQ ID NO:8. In some embodiments, the CMV promoter comprises the sequence of SEQ ID NO:6. In some embodiments, any of the constructs described herein comprises or consists of an EFS promoter of SEQ ID NO:8.

In some embodiments, the promoter comprises at least 90%, e.g., 90%, 91%, 92%, 93%, 94%, 95%, 96%, 97%, 98%, 99%, 99.9%, or more sequence identity to a murine stem cell virus (MSCV) promoter. In some embodiments, the promoter comprises a MSCV promoter. In some embodiments, the MSCV promoter comprises the sequence of SEQ ID NO:9. In some embodiments, any of the constructs described herein comprises or consists of a MSCV promoter of SEQ ID NO:9.

In some embodiments, the promoter comprises at least 90%, e.g., 90%, 91%, 92%, 93%, 94%, 95%, 96%, 97%, 98%, 99%, 99.9%, or more sequence identity to a phosphoglycerate kinase (PGK) promoter. In some embodiments, the promoter comprises a PGK promoter. In some embodiments, the PGK promoter comprises the sequence of SEQ ID NO:10. In some embodiments, any of the constructs described herein comprises or consists of a PGK promoter of SEQ ID NO:10.

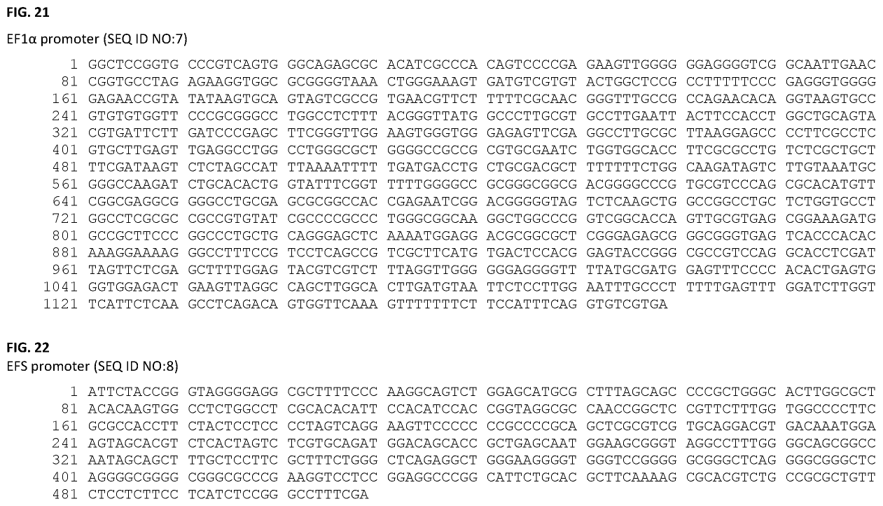

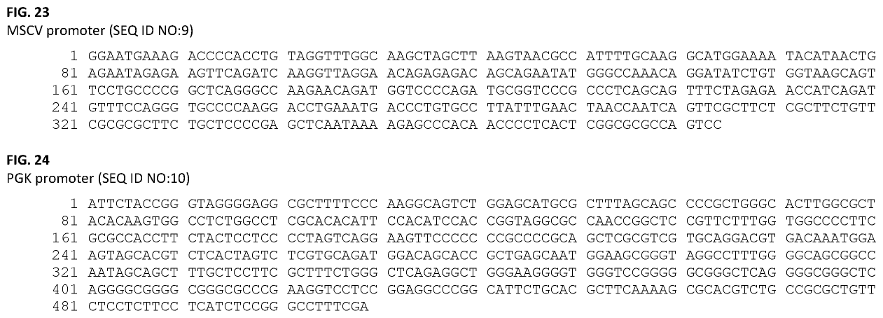

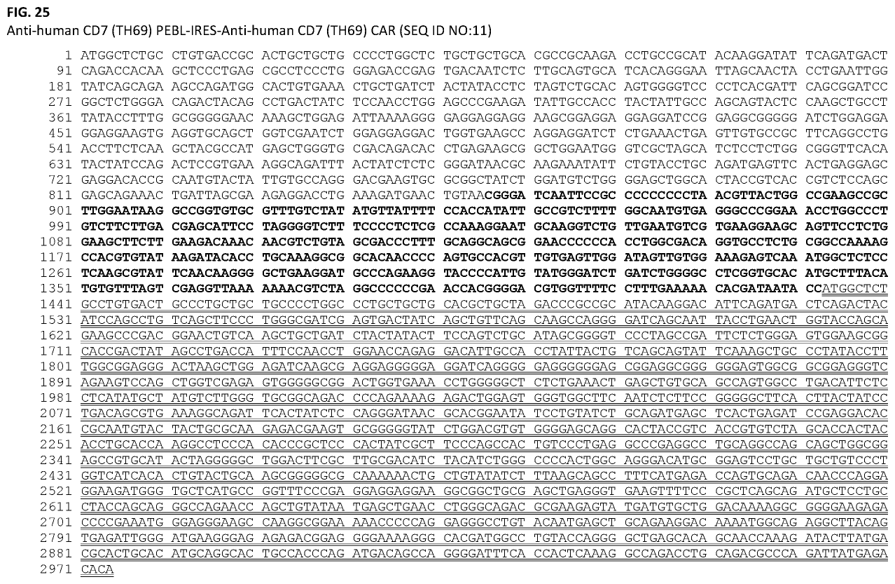

In some embodiments, the bicistronic vector comprises or consists of the nucleic acid sequence of SEQ ID NO:11. An exemplary embodiment of such a sequence is depicted in FIG. 25. In some embodiments, the bicistronic vector comprises at least 90%, e.g., 90%, 91%, 92%, 93%, 94%, 95%, 96%, 97%, 98%, 99%, 99.9%, or more sequence identity to the sequence of SEQ ID NO:11. The bicistronic vector comprises a nucleic acid sequence comprising from 5′ end to 3′ end: a nucleic acid sequence encoding a CD7 PEBL, an IRES sequence, and a nucleic acid sequence encoding a CD7 CAR, and optionally at the 5′ end, a promoter selected from the group consisting of a CMV promoter (e.g., SEQ ID NO:6 or FIG. 20), EF1a promoter (e.g., SEQ ID NO:7 or FIG. 21), EFS promoter (e.g., SEQ ID NO:8 or FIG. 22), MSCV promoter (e.g., SEQ ID NO:9 or FIG. 23), and PGK promoter (e.g., SEQ ID NO:10 or FIG. 24).

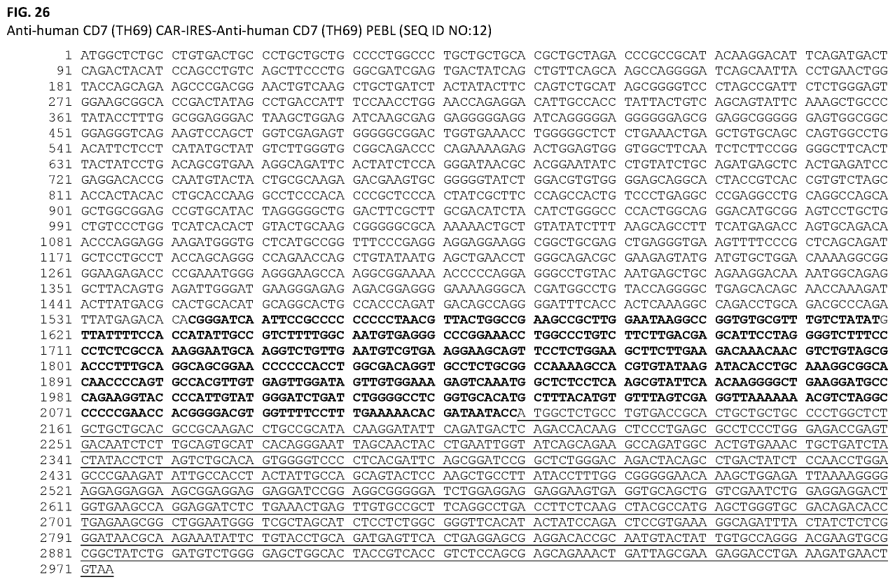

In some embodiments, the bicistronic vector comprises the nucleic acid sequence of SEQ ID NO:12. In some embodiments, the bicistronic vector comprises at least 90%, e.g., 90%, 91%, 92%, 93%, 94%, 95%, 96%, 97%, 98%, 99%, 99.9%, or more sequence identity to the sequence of SEQ ID NO:12. An exemplary embodiment of such a sequence is depicted in FIG. 26. The bicistronic vector comprises a nucleic acid sequence comprising from 5′ end to 3′ end: a nucleic acid sequence encoding a CD7 CAR, an IRES sequence, and a nucleic acid sequence encoding a CD7 PEBL, optionally at the 5′ end, a promoter selected from the group consisting of a CMV promoter, EF1a promoter, EFS promoter, MSCV promoter, and PGK promoter.

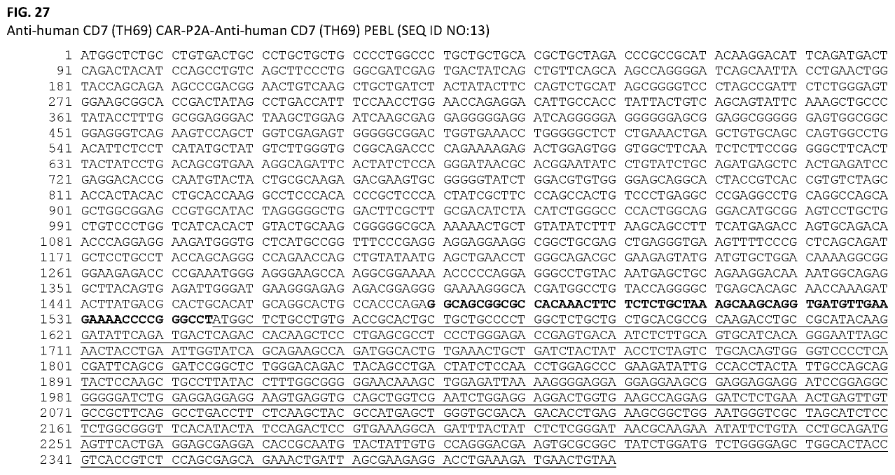

In some embodiments, the bicistronic vector comprises the nucleic acid sequence of SEQ ID NO:13. An exemplary embodiment of such a sequence is depicted in FIG. 27. In some embodiments, the bicistronic vector comprises at least 90%, e.g., 90%, 91%, 92%, 93%, 94%, 95%, 96%, 97%, 98%, 99%, 99.9%, or more sequence identity to the sequence of SEQ ID NO:13. The bicistronic vector comprises a nucleic acid sequence comprising from 5′ end to 3′ end: a nucleic acid sequence encoding a CD7 CAR, a nucleic acid sequence encoding a P2A peptide, and a nucleic acid sequence encoding a CD7 PEBL, optionally at the 5′ end, a promoter selected from the group consisting of a CMV promoter, EF1a promoter, EFS promoter, MSCV promoter, and PGK promoter.

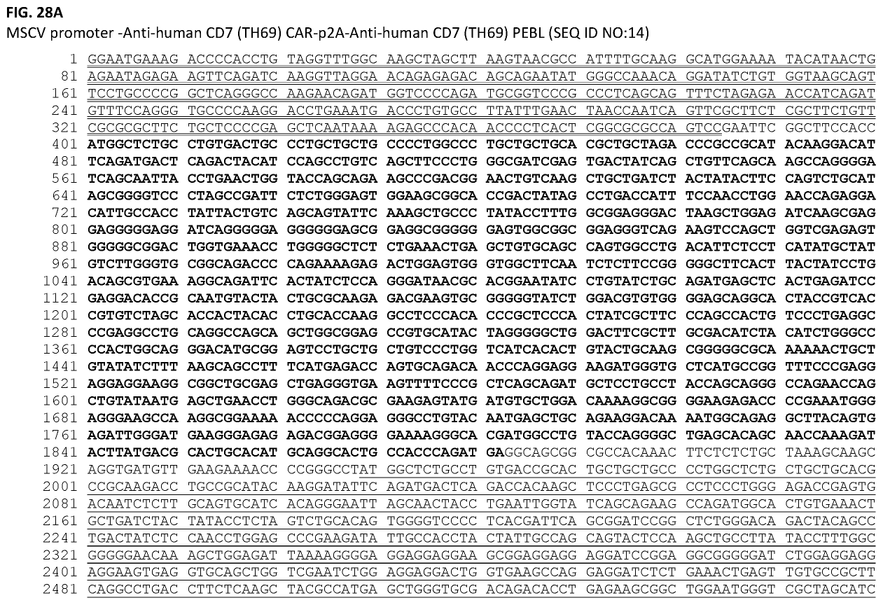

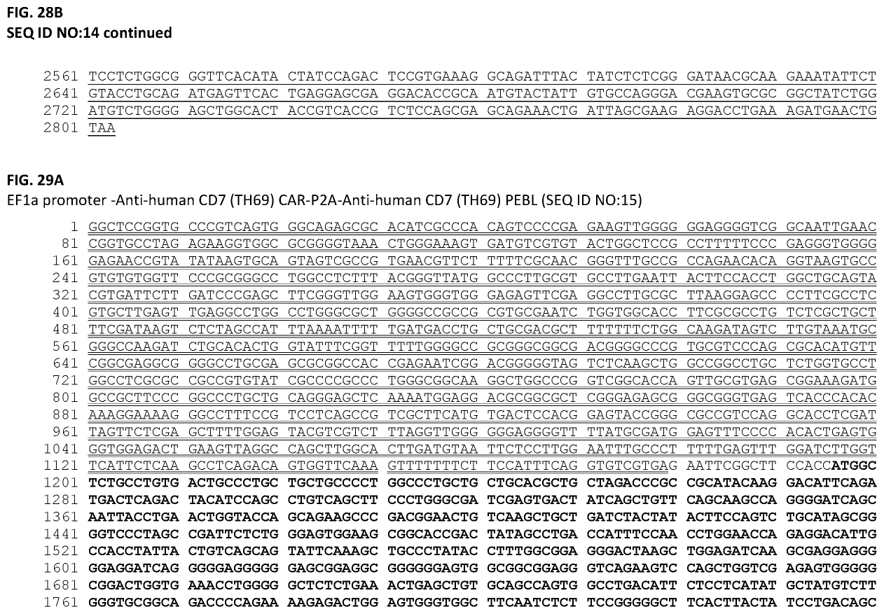

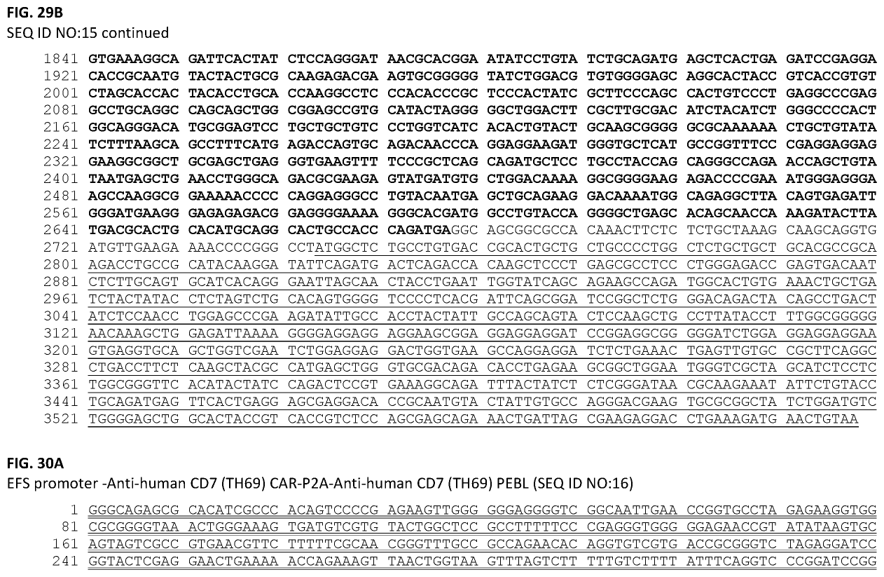

In some embodiments, the bicistronic vector comprises the nucleic acid sequence of SEQ ID NO:14. In some embodiments, the bicistronic vector comprises at least 90%, e.g., 90%, 91%, 92%, 93%, 94%, 95%, 96%, 97%, 98%, 99%, 99.9%, or more sequence identity to the sequence of SEQ ID NO:14. The bicistronic vector comprises a nucleic acid sequence comprising from 5′ end to 3′ end: a promoter, a nucleic acid sequence encoding a CD7 CAR, a P2A sequence, and a nucleic acid sequence encoding a CD7 PEBL. In some instances the bicistronic vector comprises a nucleic acid sequence comprising from 5′ end to 3′ end: a MSCV promoter, a nucleic acid sequence encoding a CD7 CAR, a nucleic acid sequence encoding a P2A peptide, and a nucleic acid sequence encoding a CD7 PEBL. An exemplary embodiment of such a sequence is depicted in FIG. 28A-FIG. 28B.

In some embodiments, the bicistronic vector comprises the nucleic acid sequence of SEQ ID NO:15. In some embodiments, the bicistronic vector comprises at least 90%, e.g., 90%, 91%, 92%, 93%, 94%, 95%, 96%, 97%, 98%, 99%, 99.9%, or more sequence identity to the sequence of SEQ ID NO:15. In some instances the bicistronic vector comprises a nucleic acid sequence comprising from 5′ end to 3′ end: an EF1α promoter, a nucleic acid sequence encoding a CD7 CAR, a nucleic acid sequence encoding a P2A peptide, and a nucleic acid sequence encoding a CD7 PEBL. An exemplary embodiment of such a sequence is depicted in FIG. 29A-FIG. 29B.

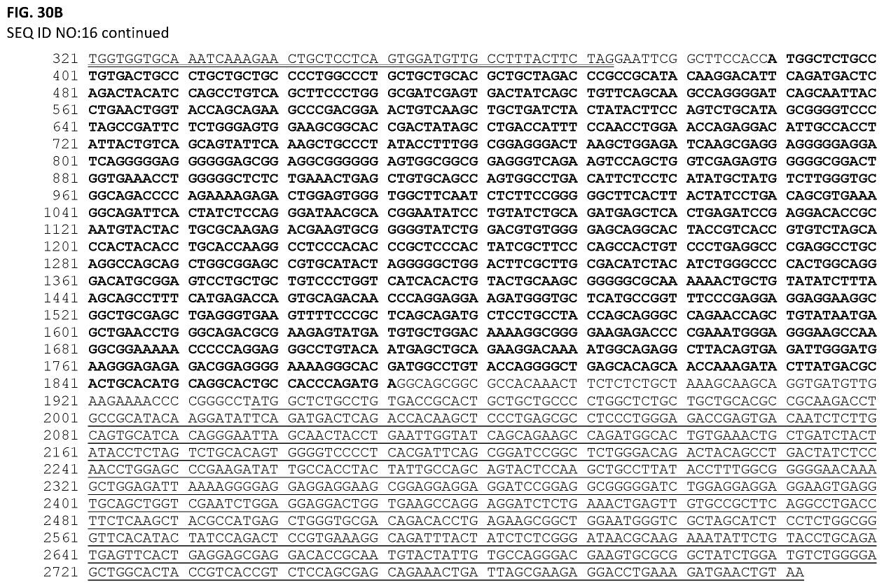

In some embodiments, the bicistronic vector comprises the nucleic acid sequence of SEQ ID NO:16. In some embodiments, the bicistronic vector comprises at least 90%, e.g., 90%, 91%, 92%, 93%, 94%, 95%, 96%, 97%, 98%, 99%, 99.9%, or more sequence identity to the sequence of SEQ ID NO:16. In some instances the bicistronic vector comprises a nucleic acid sequence comprising from 5′ end to 3′ end: an EFS promoter, a nucleic acid sequence encoding a CD7 CAR, a nucleic acid sequence encoding a P2A peptide, and a nucleic acid sequence encoding a CD7 PEBL. An exemplary embodiment of such a sequence is depicted in FIG. 30A-FIG. 30B.

### Dual Promoter Retroviral Constructs

Provided herein are recombinant retroviral constructs (or vectors) for simultaneous expression of a CAR and a PEBL in a cell such as a T cell. In some embodiments, the retroviral constructs include a promoter operably linked to a polynucleotide encoding any of the CARs described herein and a promoter operably linked to a polynucleotide encoding any of the PEBLs described herein. In some embodiments, the promoter for the CAR and the promoter for the PEBL share less than 90% sequence identity, e.g., less than 90% identity, less than 80% identity, less than 75% sequence identity, less 70% sequence identity, less than 65% sequence identity, less than 60% sequence identity, less than 55% sequence identity, and the like. In some embodiments, the promoter for the CAR and the promoter for the PEBL share 80% sequence identity or less, e.g., 80% identity, 75% sequence identity, 70% sequence identity, 65% sequence identity, 60% sequence identity, 55% sequence identity, and the like. In some embodiments, the promoter for the CAR and the promoter for the PEBL share at least 50% sequence identity, e.g., 50% sequence identity, 55% sequence identity, 60% sequence identity, 65% sequence identity, 70% sequence identity, 75% sequence identity, 80% sequence identity, 85% sequence identity, 90% sequence identity, 95% sequence identity, or more sequence identity.

In some embodiments, the promoter for the CAR (referred to as the first promoter) is different than the promoter for the PEBL (referred to as the second promoter). The first promoter and the second promoter can have the same sequence. In other instances, the first promoter and the second promoter have different sequences.

In some embodiments, the first promoter and/or second promoter comprises at least 90%, e.g., 90%, 91%, 92%, 93%, 94%, 95%, 96%, 97%, 98%, 99%, 99.9%, or more sequence identity to a CMV promoter. In some embodiments, the first promoter and/or second promoter comprises a CMV promoter. In some embodiments, the CMV promoter comprises the sequence of SEQ ID NO:6.

In some embodiments, the first promoter and/or second promoter comprises at least 90%, e.g., 90%, 91%, 92%, 93%, 94%, 95%, 96%, 97%, 98%, 99%, 99.9%, or more sequence identity to an EF1α promoter. In some embodiments, the first promoter and/or second promoter comprises an EF1α promoter. In some embodiments, the EF1α promoter comprises the sequence of SEQ ID NO:7.

In some embodiments, the first promoter and/or second promoter comprises at least 90%, e.g., 90%, 91%, 92%, 93%, 94%, 95%, 96%, 97%, 98%, 99%, 99.9%, or more sequence identity to an EFS promoter. In some embodiments, the first promoter and/or second promoter comprises an EFS promoter. In some embodiments, the EFS promoter comprises the sequence of SEQ ID NO:8.

In some embodiments, the first promoter and/or second promoter comprises at least 90%, e.g., 90%, 91%, 92%, 93%, 94%, 95%, 96%, 97%, 98%, 99%, 99.9%, or more sequence identity to a murine stem cell virus (MSCV) promoter. In some embodiments, the first promoter and/or second promoter comprises a MSCV promoter. In some embodiments, the MSCV promoter comprises the sequence of SEQ ID NO:9.

In some embodiments, the first promoter and/or second promoter comprises at least 90%, e.g., 90%, 91%, 92%, 93%, 94%, 95%, 96%, 97%, 98%, 99%, 99.9%, or more sequence identity to a phosphoglycerate kinase (PGK) promoter. In some embodiments, the first promoter and/or second promoter comprises a PGK promoter. In some embodiments, the PGK promoter comprises the sequence of SEQ ID NO:10.

In some embodiments, the retroviral constructs from 5′ to 3′ include the first promoter operably linked to the polynucleotide encoding the CAR and the second promoter operably linked to the polynucleotide encoding the PEBL. In various embodiments, the retroviral constructs from 5′ to 3′ include the second promoter operably linked to the polynucleotide encoding the PEBL and the first promoter operably linked to the polynucleotide encoding the CAR.

In some embodiments, the first promoter is located upstream of the second promoter. In some embodiments, the first promoter is a CMV promoter and the second promoter is an EFS promoter. In some embodiments, the first promoter is a CMV promoter and the second promoter is an EF1α promoter. In some embodiments, the first promoter is a CMV promoter and the second promoter is a PGK promoter. In some embodiments, the first promoter is a CMV promoter and the second promoter is a MSCV promoter. In some embodiments, the first promoter is a CMV promoter and the second promoter is a CMV promoter. In some embodiments, the first promoter is a MSCV promoter and the second promoter is an EFS promoter. In some embodiments, the first promoter is a MSCV promoter and the second promoter is an EF1α promoter. In some embodiments, the first promoter is a MSCV promoter and the second promoter is a PGK promoter. In some embodiments, the first promoter is a MSCV promoter and the second promoter is a CMV promoter. In some embodiments, the first promoter is a MSCV promoter and the second promoter is a MSCV promoter. In some embodiments, the first promoter is a PGK promoter and the second promoter is an EFS promoter. In some embodiments, the first promoter is a PGK promoter and the second promoter is an EF1α promoter. In some embodiments, the first promoter is a PGK promoter and the second promoter is a MSCV promoter. In some embodiments, the first promoter is a PGK promoter and the second promoter is a CMV promoter. In some embodiments, the first promoter is a PGK promoter and the second promoter is a PGK promoter. In some embodiments, the first promoter is an EF1α promoter and the second promoter is an MSCV promoter. In some embodiments, the first promoter is an EF1α promoter and the second promoter is an PGK promoter. In some embodiments, the first promoter is an EF1α promoter and the second promoter is an EFS promoter. In some embodiments, the first promoter is an EF1α promoter and the second promoter is a CMV promoter. In some embodiments, the first promoter is an EF1α promoter and the second promoter is an EF1α promoter. In some embodiments, the first promoter is an EFS promoter and the second promoter is an MSCV promoter. In some embodiments, the first promoter is an EFS promoter and the second promoter is an EF1α promoter. In some embodiments, the first promoter is an EFS promoter and the second promoter is an PGK promoter. In some embodiments, the first promoter is an EFS promoter and the second promoter is a CMV promoter. In some embodiments, the first promoter is an EFS promoter and the second promoter is an EFS promoter.

In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:17. In some embodiments, the retroviral construct of the present invention comprises the nucleic acid sequence of SEQ ID NO:17. In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:18. In some embodiments, the retroviral construct of the present invention comprises the nucleic acid sequence of SEQ ID NO:18. In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:19. In some embodiments, the retroviral construct of the present invention comprises the nucleic acid sequence of SEQ ID NO:19. In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:20. In some embodiments, the retroviral construct of the present invention comprises the nucleic acid sequence of SEQ ID NO:20. In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:21. In some embodiments, the retroviral construct of the present invention comprises the nucleic acid sequence of SEQ ID NO:21. In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:22. In some embodiments, the retroviral construct of the present invention comprises the nucleic acid sequence of SEQ ID NO:22. In some embodiments, the retroviral construct of the present invention comprises a nucleic acid sequence having at least 90%, e.g., 90%, 91%, 92%, 93%, 94%, 95%, 96%, 97%, 98%, 99%, 99.9%, or more sequence identity to SEQ ID NO:23. In some embodiments, the retroviral construct of the present invention comprises the nucleic acid sequence of SEQ ID NO:23.

Antibodies that Bind CD7

In certain embodiments, the anti-CD7 scFv based on the TH69 antibody comprises a variable heavy chain (heavy chain variable region or VH) and a variable light chain (light chain variable region or VL) having an amino acid sequence that each have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NOS:32 and 33, respectively. The heavy chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH sequence of SEQ ID NO:32. The light chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VL sequence of SEQ ID NO:33. In some instances, the heavy chain variable region comprises at least one (e.g., 1, 2, 3, 4, 5, 6, 7, 8, 9, 10, or more) amino acid substitution in the sequence set forth in SEQ ID NO:32. In certain instances, the heavy chain variable region comprises 10 or fewer amino acid (e.g., 0, 1, 2, 3, 4, 5, 6, 7, 8, 9, or 10) substitutions in the sequence set forth in SEQ ID NO:32. In some instances, the light chain variable region comprises at least one (e.g., 1, 2, 3, 4, 5, 6, 7, 8, 9, 10, or more) amino acid substitution in the sequence set forth in SEQ ID NO:33. In certain instances, the light chain variable region comprises 10 or fewer amino acid (e.g., 0, 1, 2, 3, 4, 5, 6, 7, 8, 9, or 10) substitutions in the sequence set forth in SEQ ID NO:33. Any of the amino acid substitutions described herein can be conservative or non-conservative substitutions.

In some embodiments, the anti-CD7 scFv comprises a VL CDR1 of SEQ ID NO:44 (SASQGISNYLN), a VL CDR2 of SEQ ID NO:45 (YTSSLHS), and a VL CDR3 of SEQ ID NO:46 (QQYSKLPYT). In some embodiments, the anti-CD7 scFv comprises a VH CDR1 of SEQ ID NO:47 (SYAMS), a VH CDR2 of SEQ ID NO:48 (SISSGGFTYYPDSVKG), and a VH CDR3 of SEQ ID NO:49 (DEVRGYLDV). In some embodiments, the anti-CD7 scFv comprises a VL CDR1 of SEQ ID NO:44, a VL CDR2 of SEQ ID NO:45, a VL CDR3 of SEQ ID NO:46, a VH CDR1 of SEQ ID NO:47, a VH CDR2 of SEQ ID NO:48, and a VH CDR3 of SEQ ID NO:49.

In some embodiments, the nucleic acid sequence encoding the VH comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the nucleic acid sequence set forth in SEQ ID NO:38. In other embodiments, the nucleic acid sequence encoding the VL comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the nucleic acid sequence set forth in SEQ ID NO:39.

In certain embodiments, the anti-CD7 scFv based on the 3A1F antibody comprises a variable heavy chain (heavy chain variable region or VH) and a variable light chain (light chain variable region or VL) having a sequence that each have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NOS:34 and 35, respectively. The heavy chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH sequence of SEQ ID NO:34. The light chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VL sequence of SEQ ID NO:35. In some instances, the heavy chain variable region comprises at least one (e.g., 1, 2, 3, 4, 5, 6, 7, 8, 9, 10, or more) amino acid substitution in the sequence set forth in SEQ ID NO:34. In certain instances, the heavy chain variable region comprises 10 or fewer amino acid (e.g., 0, 1, 2, 3, 4, 5, 6, 7, 8, 9, 10) substitutions in the sequence set forth in SEQ ID NO:34. In some cases, the light chain variable region comprises at least one (e.g., 1, 2, 3, 4, 5, 6, 7, 8, 9, or more) amino acid substitution in the sequence set forth in SEQ ID NO:35. In certain cases, the heavy chain variable region comprises 10 or fewer amino acid (e.g., 0, 1, 2, 3, 4, 5, 6, 7, 8, 9, 10) substitutions in the sequence set forth in SEQ ID NO:35. Any of the amino acid substitutions described herein can be conservative or non-conservative substitutions.

In some embodiments, the anti-CD7 scFv comprises a VL CDR1 of SEQ ID NO:50 (RASQSISNNLH), a VL CDR2 of SEQ ID NO:51 (SASQSIS), and a VL CDR3 of SEQ ID NO:52 (QQSNSWPYT). In some embodiments, the anti-CD7 scFv comprises a VH CDR1 of SEQ ID NO:53 (SYWMH), a VH CDR2 of SEQ ID NO:54 (KINPSNGRTNYNEKFKS), and a VH CDR3 of SEQ ID NO:55 (GGVYYDLYYYALDY). In various embodiments, the anti-CD7 scFv comprises a VL CDR1 of SEQ ID NO:50, a VL CDR2 of SEQ ID NO:51, a VL CDR3 of SEQ ID NO:52, a VH CDR1 of SEQ ID NO:53, a VH CDR2 of SEQ ID NO:54, and a VH CDR3 of SEQ ID NO:55.

In some embodiments, the nucleic acid sequence encoding the VH comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the nucleic acid sequence set forth in SEQ ID NO:40. In other embodiments, the nucleic acid sequence encoding a VL comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the nucleic acid sequence set forth in SEQ ID NO:41.

In certain embodiments, the anti-CD7 scFv based on the T3-3A1 antibody comprises a variable heavy chain (heavy chain variable region or VH) and a variable light chain (light chain variable region or VL) having a sequence that each have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NOS:36 and 37, respectively. The heavy chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH sequence of SEQ ID NO:36. The light chain variable region can comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VL sequence of SEQ ID NO:37. In some instances, the heavy chain variable region comprises at least one (e.g., 1, 2, 3, 4, 5, 6, 7, 8, 9, 10, or more) amino acid substitution in the sequence set forth in SEQ ID NO:36. In certain instances, the heavy chain variable region comprises 13 or fewer amino acid (e.g., 0, 1, 2, 3, 4, 5, 6, 7, 8, 9, 10, 11, 12, or 13) substitutions in the sequence set forth in SEQ ID NO:36. In some cases, the light chain variable region comprises at least one (e.g., 1, 2, 3, 4, 5, 6, 7, 8, 9, 10, or more) amino acid substitution in the sequence set forth in SEQ ID NO:37. In certain cases, the heavy chain variable region comprises 5 or fewer amino acid (e.g., 0, 1, 2, 3, 4, 5, 6, 7, 8, 9, 10, or 11) substitutions in the sequence set forth in SEQ ID NO:37. Any of the amino acid substitutions described herein can be conservative or non-conservative substitutions.

In some embodiments, the anti-CD7 scFv comprises a VL CDR1 of SEQ ID NO:56 (RASKSVSASGYSYMH), a VL CDR2 of SEQ ID NO:57 (LASNLES), and a VL CDR3 of SEQ ID NO:58 (QHSRELPYT). In some embodiments, the anti-CD7 scFv comprises a VH CDR1 of SEQ ID NO:59 (SFGMH), a VH CDR2 of SEQ ID NO:60 (YISSGSSTLHYADTVKG), and a VH CDR3 of SEQ ID NO:61 (WGNYPHYAMDY). In various embodiments, the anti-CD7 scFv comprises a VL CDR1 of SEQ ID NO:56, a VL CDR2 of SEQ ID NO:57, a VL CDR3 of SEQ ID NO:58, a VH CDR1 of SEQ ID NO:59, a VH CDR2 of SEQ ID NO:60, and a VH CDR3 of SEQ ID NO:61.

In some embodiments, the nucleic acid sequence encoding the VH comprises at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to the nucleic acid sequence set forth in SEQ ID NO:42. In other embodiments, the nucleic acid sequence encoding the VL comprises at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to the nucleic acid sequence set forth in SEQ ID NO:43.

In some embodiments, the scFv of the present invention comprises a variable heavy chain sequence having at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to a variable heavy chain sequence of an anti-CD7 antibody. In some embodiments, the scFv of the present invention comprises a variable light chain sequence having at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to a variable light chain sequence of an anti-CD7 antibody. For instance, the anti-CD7 antibody can be any such recognized by one skilled in the art.

TABLE 3
Amino acid sequences of VII regions and VL
regions of anti-CD7 scFvs

| Anti- | Compo- | |
|---|---|---|
| body | nent | Amino Acid Sequence |
| TH69 | VH | EVQLVESGGGLVKPGGSLKLSCAASGLTFSSYAMS |
| | | WVRQTPEKRLEWVASISSGGFTYYPDSVKGRFTIS |
| | | RDNARNILYLQMSSLRSEDTAMYYCARDEVRGYLD |
| | | VWGAGTTVTVSS (SEQ ID NO: 32) |
| | VL | AAYKDIQMTQTTSSLSASLGDRVTISCSASQGISN |
| | | YLNWYQQKPDGTVKLLIYYTSSLHSGVPSRFSGSG |
| | | SGTDYSLTISNLEPEDIATYYCQQYSKLPYTFGGG |
| | | TKLEIKR (SEQ ID NO: 33) |
| 3A1F | VH | QVQLQESGAELVKPGASVKLSCKASGYTFTSYWMH |
| | | WVKQRPGQGLEWIGKINPSNGRTNYNEKFKSKATL |
| | | TVDKSSSTAYMQLSSLTSEDSAVYYCARGGVYYDL |
| | | YYYALDYWGQGTTVTVSS (SEQ ID NO: 34) |
| | VL | DIELTQSPATLSVTPGDSVSLSCRASQSISNNLHW |
| | | YQQKSHESPRLLIKSASQSISGIPSRFSGSGSGTD |
| | | FTLSINSVETEDFGMYFCQQSNSWPYTFGGGTKLE |
| | | IKR (SEQ ID NO: 35) |
| T3-3A1 | VH | DVQLVESGGGLVQPGGSRKLSCAASGFTFSSFGMH |
| | | WVRQAPEKGLEWVAYISSGSSTLHYADTVKGRFTI |
| | | SRDNPKNTLFLQMTSLRSEDTAMYYCARWGNYPHY |
| | | AMDYWGQGTSVTVSS (SEQ ID NO: 36) |
| | VL | DIVMTQSPASLAVSLGQRATISCRASKSVSASGYS |
| | | YMHWYQQKPGQPPKLLIYLASNLESGVPARFSGSG |
| | | SGTDFTLNIHPVEEEDAVTYYCQHSRELPYTFGGG |
| | | TKLEIK (SEQ ID NO: 37) |

TABLE 4
Nucleic acid sequences of VH regions and VL
regions of anti-CD7 scFvs

| Anti- | Compo- | |
|---|---|---|
| body | nent | Nucleic Acid Sequence |
| TH69 | VH | GAGGTGCAGCTGGTCGAATCTGGAGGAGGACTGGT |
| | | GAAGCCAGGAGGATCTCTGAAACTGAGTTGTGCCG |
| | | CTTCAGGCCTGACCTTCTCAAGCTACGCCATGAGC |
| | | TGGGTGCGACAGACACCTGAGAAGCGGCTGGAATG |
| | | GGTCGCTAGCATCTCCTCTGGCGGGTTCACATACT |
| | | ATCCAGACTCCGTGAAAGGCAGATTTACTATCTCT |
| | | CGGGATAACGCAAGAAATATTCTGTACCTGCAGAT |
| | | GAGTTCACTGAGGAGCGAGGACACCGCAATGTACT |
| | | ATTGTGCCAGGGACGAAGTGCGCGGCTATCTGGAT |
| | | GTCTGGGGAGCTGGCACTACCGTCACCGTCTCCAG |
| | | C |
| | | (SEQ ID NO: 38) |
| | VL | GCCGCATACAAGGATATTCAGATGACTCAGACCAC |
| | | AAGCTCCCTGAGCGCCTCCCTGGGAGACCGAGTGA |
| | | CAATCTCTTGCAGTGCATCACAGGGAATTAGCAAC |
| | | TACCTGAATTGGTATCAGCAGAAGCCAGATGGCAC |
| | | TGTGAAACTGCTGATCTACTATACCTCTAGTCTGC |
| | | ACAGTGGGGTCCCCTCACGATTCAGCGGATCCGGC |
| | | TCTGGGACAGACTACAGCCTGACTATCTCCAACCT |
| | | GGAGCCCGAAGATATTGCCACCTACTATTGCCAGC |
| | | AGTACTCCAAGCTGCCTTATACCTTTGGCGGGGGA |
| | | ACAAAGCTGGAGATTAAAAGG |
| | | (SEQ ID NO: 39) |
| 3A1F | VH | CAGGTCCAGCTGCAGGAGTCAGGAGCTGAGCTGGT |
| | | GAAGCCAGGGGCAAGCGTCAAACTGTCCTGCAAGG |
| | | TCCTCGGATATACATTCACTAGCTACTGGATGCAC |
| | | TGGGTGAAACAGAGACCCGGACAGGGCCTGGAGTG |
| | | GATCGGAAAGATTAACCCTAGCAATGGCAGGACCA |
| | | ACTACAACGAAAAGTTTAAATCCAAGGCAACCCTG |
| | | ACAGTGGACAAGAGCTCCTCTACAGCCTACATGCA |
| | | GCTGAGTTCACTGACTTCAGAGGATAGCGCAGTGT |
| | | ACTATTGCGCCAGAGGCGGGGTCTACTATGACCTG |
| | | TACTATTACGCCCTGGATTATTGGGGGCAGGGAAC |
| | | CACAGTGACTGTCAGCTCC |
| | | (SEQ ID NO: 40) |
| | VL | GACATCGAGCTGACCCAGAGTCCTGCTACACTGAG |
| | | CGTGACTCCAGGCGATTCTGTCAGTCTGTCATGTC |
| | | GGGCAAGCCAGTCCATCTCTAACAATCTGCACTGG |
| | | TACCAGCAGAAATCCCATGAATCTCCACGACTGCT |
| | | GATTAAGAGTGCCTCACAGAGCATCTCCGGCATTC |
| | | CCTCCCGGTTCTCTGGCAGTGGGTCAGGAACTGAC |
| | | TTTACCCTGAGTATTAACTCAGTGGAGACAGAAGA |
| | | TTTCGGCATGTATTTTTGCCAGCAGAGCAATTCCT |
| | | GGCCCTACACTTTCGGAGGCGGGACCAAACTGGAG |
| | | ATCAAGCGG |
| | | (SEQ ID NO: 41) |
| T3-3A1 | VH | GATGTGCAGCTGGTGGAGTCTGGGGGAGGCTTAGT |
| | | GCAGCCTGGAGGGTCCCGGAAACTCTCCTGTGCAG |
| | | CCTCTGGATTCACTTTCAGTAGCTTTGGAATGCAC |
| | | TGGGTTCGTCAGGCTCCAGAGAAGGGGCTGGAGTG |
| | | GGTCGCATACATTAGTAGTGGCAGTAGTACCCTCC |
| | | ACTATGCAGACACAGTGAAGGGCCGATTCACCATC |
| | | TCCAGAGACAATCCCAAGAACACCCTGTTCCTGCA |
| | | AATGACCAGTCTAAGGTCTGAGGACACGGCCATGT |
| | | ATTACTGTGCAAGATGGGGTAACTACCCTCACTAT |
| | | GCTATGGACTACTGGGGTCAAGGAACCTCAGTCAC |
| | | CGTCTCCTCA |
| | | SEQ ID NO: 42) |
| | VL | GACATTGTGATGACCCAGTCTCCTGCTTCCTTAGC |
| | | TGTATCTCTGGGGCAGAGGGCCACCATCTCATGCA |
| | | GGGCCAGCAAAAGTGTCAGTGCATCTGGCTATAGT |
| | | TATATGCACTGGTACCAACAGAAACCAGGACAGCC |
| | | ACCCAAACTCCTCATCTATCTTGCATCCAACCTAG |
| | | AATCTGGGGTCCCTGCCAGGTTCAGTGGCAGTGGG |
| | | TCTGGGACAGACTTCACCCTCAACATCCATCCTGT |
| | | GGAGGAGGAGGATGCTGTAACCTATTACTGTCAGC |
| | | ACAGTAGGGAGCTTCCGTACACGTTCGGAGGGGGG |
| | | ACCAAGCTGGAAATAAAA |
| | | (SEQ ID NO: 43) |

### Downregulation of Intracellular CD7 Via CD7 PEBL

As described herein, T cell cytotoxicity was shown to be markedly increased when anti-CD7 CAR was used in combination with downregulation of CD7 expression on the effector T cells. As demonstrated herein, downregulation (e.g., elimination, reduction, and/or relocalization) of CD7 prevented the fratricidal effect exerted by the corresponding anti-CD7 CAR, allowing greater T cell recovery after CAR expression as compared to cells that retained the target antigen (e.g., CD7), and a more effective cytotoxicity against T leukemia/lymphoma cells. As those of skill in the art would appreciate, downregulation of CD7 expression on the effector T cells can be achieved according to a variety of known methods including, for example, protein expression blockers (PEBLs) against CD7 (as described in WO2016/126213), RNAi against CD7, or gene editing methods such as, e.g., meganucleases, TALEN, CRISPR/Cas9, and zinc finger nucleases. The present invention describes PEBLs that bind target antigens and sequester the target antigens to the cytoplasm of a cell. The target antigens are synthesized and bind to the PEBLs intracellularly.

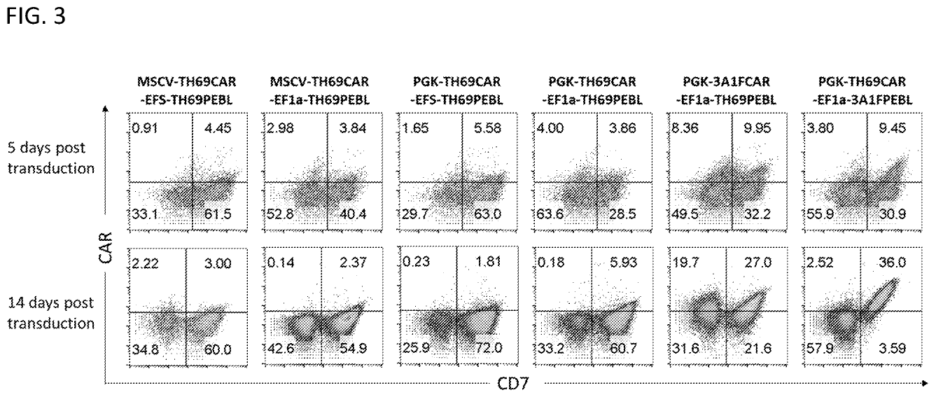

In certain embodiments, provided herein is a polynucleotide comprising a nucleic acid sequence encoding a PEBL comprising a target-binding molecule (e.g., a CD7 antigen binding domain) linked to a localizing domain. In some instances, the PEBL comprises from the N-terminus to the C-terminus: a CD7 antigen binding domain, an optional domain linker, and a cellular localizing domain. In some embodiments, the PEBL further comprises a signal peptide fused N-terminal to the CD7 antigen binding domain. In some embodiments, the CD7 antigen binding domain comprises a VL domain, a domain linker, and a VH domain. Exemplary embodiments of a PEBL are shown in FIG. 3E and FIG. 17 of US 2018/0179280, which is herein incorporated by reference.

As used herein, “linked” in the context of the protein expression blocker refers to a gene encoding a target-binding molecule directly in frame (e.g., without a linker) adjacent to one or more genes encoding one or more localizing domains. Alternatively, the gene encoding a target-binding molecule may be connected to one or more gene encoding one or more localizing domains through a linker sequence, e.g., as described in WO2016/126213. As would be appreciated by those of skill in the art, such linker sequences as well as variants of such linker sequences are known in the art. Methods of designing constructs that incorporate linker sequences as well as methods of assessing functionality are readily available to those of skill in the art.

In some embodiments, the localizing domain of the PEBL comprises an endoplasmic reticulum (ER) or Golgi retention sequence; or a proteosome localizing sequence. In certain embodiments, the localizing domain comprises an endoplasmic reticulum (ER) retention peptide of Table 5. In certain embodiments, the localizing domain comprises a proteasome localizing sequence set forth in Table 5. The localizing domain can direct the PEBL to a specific cellular compartment, such as the Golgi or endoplasmic reticulum, the proteasome, or the cell membrane, depending on the application.

In some embodiments, proteasome localization is achieved by linking the scFv sequence to a tripartite motif containing 21 (TRIM21) targeting domain sequence and coexpressing the sequence encoding the human TRIM21 E3 ubiquitin ligase protein. TRIM21 binds with high affinity to the Fc domains of antibodies and can recruit the ubiquitin-proteosome complex to degrade molecules (e.g., proteins and peptides) bound to the antibodies. The TRIM21 targeting domain sequence encodes amino acid sequences selected from the group of human immunoglobulin G (IgG) constant regions (Fc) genes such as IgG1, IgG2, or IgG4 and is used to form a fusion protein comprising scFv and Fc domains. In this embodiment, the exogenously expressed TRIM21 protein binds the scFv-Fc fusion protein bound to the target protein (e.g., CD7) and directs the complex to the proteasome for degradation.

Details of the amino acid sequence of the human TRIM21 E3 ligase protein can be found, for example, in NCBI Protein database under NCBI Ref. Seq. No. NP_003132.2. Details of the nucleic acid sequence encoding the human TRIM21 E3 ligase protein can be found, for example, in NCBI Protein database under NCBI Ref. Seq. No. NM_003141.3.

In some embodiments, the PEBL also includes a hinge domain and transmembrane domain sequence derived from CD8a, CD80, 4-1BB, CD28, CD34, CD4, FcεRIγ, CD16, OX40, CD3ζ, CD3ε, CD3γ, CD3δ, TCRα, CD32, CD64, VEGFR2, FAS, or FGFR2B. In some embodiments, the PEBL comprises a hinge and transmembrane domain selected from the group consisting of a hinge and transmembrane domain of CD8α, a hinge and transmembrane domain of CD80, a hinge and transmembrane domain of 4-1BB, a hinge and transmembrane domain of CD28, a hinge and transmembrane domain of CD34, a hinge and transmembrane domain of CD4, a hinge and transmembrane domain of FcεRIγ, a hinge domain and transmembrane domain of CD16, a hinge and transmembrane domain of OX40, a hinge and transmembrane domain of CD3ζ, a hinge and transmembrane domain of CD3ε, a hinge and transmembrane domain of CD3γ, a hinge and transmembrane domain of CD3δ, a hinge and transmembrane domain of TCRα, a hinge and transmembrane domain of CD32, a hinge and transmembrane domain of CD64, a hinge and transmembrane domain of VEGFR2, a hinge and transmembrane domain of FAS, and a hinge and transmembrane domain of FGFR2B.

In certain embodiments, the PEBL comprises one or more of the components set forth in Table 5.

TABLE 5
Amino acid sequence information for
select components of a CD7 PEBL

| | SEQ ID | |
|---|---|---|
| Component | NO | Amino Acid Sequence |
| ER locali- | SEQ ID | EQKLISEEDLKDEL |
| zation | NO: 71 | |
| domain | | |
| KDEL | | |
| tethered | | |
| to scFv | | |
| with myc | | |
| (“myc | | |
| KDEL”) | | |
| Localiza- | SEQ ID | GGGGSGGGGSGGGGSGGGG |
| tion domain | NO: 72 | SAEKDEL |
| “link(20) | | |
| AEKDEL” | | |
| KDEL | SEQ ID | KDEL |
| domain | NO: 73 | |
| KKXX domain | SEQ ID | KKXX where X is any |
| | NO: 74 | amino acid |
| KXD/E | | KXD or KXE where X |
| domain | | is any amino acid |
| YQRL domain | SEQ ID | YQRL |
| | NO: 75 | |
| PEST motif | SEQ ID | PEST |
| | NO: 76 | |
| Localiza- | SEQ ID | TTTPAPRPPTPAPTIASQP |
| tion domain | NO: 77 | LSLRPEACRPAAGGAVHTR |
| “mb DEKKMP” | | GLDFACDIYIWAPLAGTCG |
| domain | | VLLLSLVITLYKYKSRRSF |
| | | IDEKKMP |
| CD8α hinge | SEQ ID | TTTPAPRPPTPAPTIASQP |
| and trans- | NO: 78 | LSLRPEACRPAAGGAVHTR |
| membrane | | GLDFACDIYIWAPLAGTCG |
| domain | | VLLLSLVITLY |
| VH-VL | SEQ ID | GGGGSGGGGSGGGGSGGGGS |
| linker | NO: 79 | |
| CD8α | SEQ ID | MALPVTALLLPLALLLHAARP |
| signal | NO: 80 | |
| peptide | | |

In some embodiments, the CD7 PEBL contains CD7 antigen binding domain comprising an amino acid sequence of SEQ ID NO:32, an amino acid sequence of SEQ ID NO:33, and a VH-VL linker. The VH-VL linker can be a (G₄S)ₙ linker where n can range from 1 to 6, e.g., 1, 2, 3, 4, 5, or 6. In one embodiment, the CD7 PEBL comprises an amino acid sequence of SEQ TD NO:32, an amino acid sequence of SEQ TD NO:33, and an amino acid sequence of SEQ ID NO:79. In some embodiments, the CD7 PEBL comprises an amino acid sequence having at least 9000 sequence identity or at least 9500 sequence identity to SEQ ID NO:32, the amino acid sequence of SEQ ID NO:33, and the amino acid sequence of SEQ TD NO:79. In certain embodiments, the CD7 PEBL comprises an amino acid sequence of SEQ ID NO:32, an amino acid sequence having at least 9000 sequence identity or at least 9500 sequence identity to SEQ ID NO: 33, and an amino acid sequence of SEQ ID NO:79. In other embodiments, the anti-CD7 protein expression blocker comprises an amino acid sequence having at least 90% sequence identity or at least 95% sequence identity to SEQ ID NO:32, an amino acid sequence having at least 90% sequence identity or at least 95% sequence identity to SEQ ID NO:33, and an amino acid sequence of SEQ ID NO:79.

In some embodiments, the CD7 PEBL contains CD7 antigen binding domain comprising an amino acid sequence of SEQ ID NO:34, an amino acid sequence of SEQ ID NO:35, and a VH-VL linker. The VH-VL linker can be a (G₄S)ₙ linker where n can range from 1 to 6, e.g., 1, 2, 3, 4, 5, or 6. In one embodiment, the CD7 PEBL comprises an amino acid sequence of SEQ ID NO:34, an amino acid sequence of SEQ ID NO:35, and an amino acid sequence of SEQ ID NO:79. In some embodiments, the CD7 PEBL comprises an amino acid sequence having at least 95% sequence identity to SEQ ID NO:34, the amino acid sequence of SEQ ID NO:35, and the amino acid sequence of SEQ ID NO:79. In certain embodiments, the CD7 PEBL comprises an amino acid sequence of SEQ ID NO:34, an amino acid sequence having at least 95% sequence identity to SEQ ID NO:35, and an amino acid sequence of SEQ ID NO:79. In other embodiments, the CD7 PEBL comprises an amino acid sequence having at least 90% sequence identity or at least 95% sequence identity to SEQ ID NO:34, an amino acid sequence having at least 90% sequence identity or at least 95% sequence identity to SEQ ID NO:35, and an amino acid sequence of SEQ ID NO:79.

In some embodiments, the CD7 PEBL contains CD7 antigen binding domain comprising an amino acid sequence of SEQ ID NO:36, an amino acid sequence of SEQ ID NO:37, and a VH-VL linker. The VH-VL linker can be a (G₄S)ₙ linker where n can range from 1 to 5, e.g., 1, 2, 3, 4, 5, or 6. In one embodiment, the CD7 PEBL comprises an amino acid sequence of SEQ ID NO:36, an amino acid sequence of SEQ ID NO:37, and an amino acid sequence of SEQ ID NO:79. In some embodiments, the CD7 PEBL comprises an amino acid sequence having at least 90% sequence identity or at least 95% sequence identity to SEQ ID NO:36, the amino acid sequence of SEQ ID NO:37, and the amino acid sequence of SEQ ID NO:79. In certain embodiments, the CD7 PEBL comprises an amino acid sequence of SEQ ID NO:36, an amino acid sequence having at least 90% sequence identity or at least 95% sequence identity to SEQ ID NO:37, and an amino acid sequence of SEQ ID NO:79. In other embodiments, the CD7 PEBL comprises an amino acid sequence having at least 90% sequence identity or at least 95% sequence identity to SEQ ID NO:36, an amino acid sequence having at least 90% sequence identity or at least 95% sequence identity to SEQ ID NO:37, and an amino acid sequence of SEQ ID NO:79.

In some instance, CD7 PEBL also comprises a localization domain selected from any one sequence set forth in SEQ ID NOS:72-77. In some cases, the CD7 PEBL also comprises a CD8a signal peptide such as but not limited to the CD8a signal peptide set forth in SEQ ID NO:80. In other cases, the anti-CD7 protein expression blocker also comprises CD8a hinge and transmembrane domains such as but not limited to the CD8a hinge and transmembrane domains set forth in SEQ ID NO:78.

In one embodiment, the CD7 PEBL encoded by the bicistronic vector described herein comprises the sequence of SEQ ID NO:24 and a proline at the N-terminus. In some embodiments, the CD7 PEBL comprises the sequence of SEQ ID NO:25. The N-terminal proline residue arises from the 2A cleavage. In some embodiments, the CD7 PEBL encoded by the bicistronic vector described herein comprises the sequence of SEQ ID NO:26 and a proline at the N-terminus. In some embodiments, the CD7 PEBL comprises the sequence of SEQ ID NO:27.

In some embodiments, an engineered immune cell of the present invention comprises a CD7 PEBL encoded by a bicistronic vector such that the CD7 PEBL comprises the sequence of SEQ ID NO:24 and a proline at the N-terminus or the sequence of SEQ ID NO:25. In some embodiments, the engineered immune cell is a CD4+ T cell comprising a CD7 PEBL encoded by a bicistronic vector such that the CD7 PEBL comprises the sequence of SEQ ID NO:24 and a proline at the N-terminus or the sequence of SEQ ID NO:25. In some embodiments, the engineered immune cell is a CD8+ T cell comprising a CD7 PEBL encoded by the bicistronic vector wherein the CD7 PEBL comprises the sequence of SEQ ID NO:24 and a proline at the N-terminus or the sequence of SEQ ID NO:25. In some embodiments, the engineered immune cell is a CD3+ T cell comprising a CD7 PEBL encoded by the bicistronic vector wherein the CD7 PEBL comprises the sequence of SEQ ID NO:24 and a proline at the N-terminus or the sequence of SEQ ID NO:25.

In some embodiments, an engineered immune cell of the present invention comprises a CD7 PEBL encoded by a bicistronic vector such that the CD7 PEBL comprises the sequence of SEQ ID NO:26 and a proline at the N-terminus or the sequence of SEQ ID NO:27. In some embodiments, the engineered immune cell is a CD4+ T cell comprising a CD7 PEBL encoded by the bicistronic vector wherein the CD7 PEBL comprises the sequence of SEQ ID NO:26 and a proline at the N-terminus or the sequence of SEQ ID NO:27. In some embodiments, the engineered immune cell is a CD8+ T cell comprising a CD7 PEBL encoded by the bicistronic vector wherein the CD7 PEBL comprises the sequence of SEQ ID NO:26 and a proline at the N-terminus or the sequence of SEQ ID NO:27. In some embodiments, the engineered immune cell is a CD3+ T cell comprising a CD7 PEBL encoded by the bicistronic vector wherein the CD7 PEBL comprises the sequence of SEQ ID NO:26 and a proline at the N-terminus or the sequence of SEQ ID NO:27.

In some embodiments, the CD7 PEBL encoded by the dual promoter vector described herein comprises the sequence of SEQ ID NO:24. In some embodiments, the CD7 PEBL encoded by the dual promoter vector described herein binds to CD7 and comprises at least 90% sequence identity to SEQ ID NO:24. In some embodiments, the CD7 PEBL encoded by the dual promoter vector described herein comprises the sequence of SEQ ID NO:26. In some embodiments, the CD7 PEBL encoded by the dual promoter vector described herein binds to CD7 and comprises at least 90% sequence identity to SEQ ID NO:26.

In some embodiments, the polynucleotide encoding the CD7 PEBL comprises one or more nucleic acid sequences set forth in Table 6.

In some embodiments, the VH domain of the anti-CD7 scFv of the PEBL comprises the nucleotide sequence of SEQ ID NO:38 and the VL domain of the anti-CD7 scFv of the PEBL comprises the nucleotide sequence of SEQ ID NO:39. In certain embodiments, the VH domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:38 and the VL domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:39. In some embodiments, the VH domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:38 and the VL domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:39, or a codon optimized variant thereof.

In some embodiments, the VH domain of the anti-CD7 scFv of the PEBL comprises the nucleotide sequence of SEQ ID NO:40 and the VL domain of the anti-CD7 scFv of the PEBL comprises the nucleotide sequence of SEQ ID NO:41. In certain embodiments, the VH domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:40 and the VL domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:41. In some embodiments, the VH domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:40 and the VL domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:41, or a codon optimized variant thereof.

In some embodiments, the VH domain of the anti-CD7 scFv of the PEBL comprises the nucleotide sequence of SEQ ID NO:42 and the VL domain of the anti-CD7 scFv of the PEBL comprises the nucleotide sequence of SEQ ID NO:43. In certain embodiments, the VH domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:42 and the VL domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:43. In some embodiments, the VH domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:42 and the VL domain of the anti-CD7 scFv of the PEBL comprises the nucleotide sequence having at least 90% sequence identity (e.g., 90%, 91%, 92%, 93%, 94%, 95%, 96%, 97%, 98%, 99%, or more sequence identity) to SEQ ID NO:43, or a codon optimized variant thereof.

TABLE 6
Nucleic acid sequence information for select
components of a CD7 PEBL

| | SEQ ID | |
|---|---|---|
| Component | NO | Sequence |
| CD8α | SEQ ID | ATGGCTCTGCCTGTGACCGCACTGCTGCTGCCC |
| signal | NO: 81 | CTGGCTCTGCTGCTGCACGCCGCAAGACCT |
| peptide | | |
| VH-VL | SEQ ID | GGAGGAGGAGGAAGCGGAGGAGGAGGATCCGGA |
| Linker | NO: 82 | GGCGGGGGATCTGGAGGAGGAGGAAGT |
| ER | SEQ ID | GAGCAGAAACTGATTAGCGAAGAGGACCTGAAA |
| localiza- | NO: 83 | GATGAACTG |
| tion | | |
| domain | | |
| KDEL | | |
| tethered | | |
| to scFv | | |
| with myc | | |
| (“myc | | |
| KDEL”) | | |

In certain aspects of the present invention, the PEBL can bind to a molecule that is expressed on the surface of a cell including, but not limited to members of the CD1 family of glycoproteins, CD2, CD3, CD4, CD5, CD7, CD8, CD25, CD28, CD30, CD38, CD45, CD45RA, CD45RO, CD52, CD56, CD57, CD99, CD127, and CD137.

In some embodiments, the CD7 PEBL comprises a nucleic acid sequence having at least 85% (e.g., 85%, 86%, 87%, 88%, 89%, 90%, or more) sequence identity to SEQ ID NO:2 and binds to CD7. In some embodiments, the CD7 PEBL comprises a nucleic acid sequence having at least 90% (e.g., 90%, 91%, 92%, 94%, 94%, 95%, 96%, 97%, 98%, 99%, or more) sequence identity to SEQ ID NO:2 and binds to CD7. In some embodiments, the CD7 PEBL comprises a nucleic acid sequence having at least 90% (e.g., 90%, 91%, 92%, 94%, 94%, 95%, 96%, 97%, 98%, 99%, or more) sequence identity to SEQ ID NO:2.

In some embodiments, the CD7 PEBL comprises a nucleic acid sequence having at least 85% (e.g., 85%, 86%, 87%, 88%, 89%, 90%, or more) sequence identity to SEQ ID NO:3 and binds to CD7. In some embodiments, the CD7 PEBL comprises a nucleic acid sequence having at least 90% (e.g., 90%, 91%, 92%, 94%, 94%, 95%, 96%, 97%, 98%, 99%, or more) sequence identity to SEQ ID NO:3 and binds to CD7. In some embodiments, the CD7 PEBL comprises a nucleic acid sequence having at least 90% (e.g., 90%, 91%, 92%, 94%, 94%, 95%, 96%, 97%, 98%, 99%, or more) sequence identity to SEQ ID NO:3.

In some embodiments, an engineered immune cell of the present invention comprises a CD7 PEBL encoded by a bicistronic construct comprising a nucleic acid sequence of the CD7 PEBL having at least 90% (e.g., 90%, 91%, 92%, 94%, 94%, 95%, 96%, 97%, 98%, 99%, or more) sequence identity to SEQ ID NO:2. In some embodiments, the engineered immune cell is a CD4+ T cell comprising a CD7 PEBL encoded by the bicistronic vector construct comprising a nucleic acid sequence of the CD7 PEBL having at least 90% sequence identity to SEQ ID NO:2. In some embodiments, the engineered immune cell is a CD8+ T cell comprising a CD7 PEBL encoded by the bicistronic construct comprising a nucleic acid sequence of the CD7 PEBL having at least 90% sequence identity to SEQ ID NO:2. In some embodiments, the engineered immune cell is a CD3+ T cell comprising a CD7 PEBL encoded by the bicistronic construct comprising a nucleic acid sequence of the CD7 PEBL having at least 90% sequence identity to SEQ ID NO:2. Also, provided herein is a population comprising such cells.

In some embodiments, an engineered immune cell of the present invention comprises a CD7 PEBL encoded by a bicistronic construct comprising a nucleic acid sequence of the CD7 PEBL having at least 90% (e.g., 90%, 91%, 92%, 94%, 94%, 95%, 96%, 97%, 98%, 99%, or more) sequence identity to SEQ ID NO:3. In some embodiments, the engineered immune cell is a CD4+ T cell comprising a CD7 PEBL encoded by the bicistronic vector construct comprising a nucleic acid sequence of the CD7 PEBL having at least 90% sequence identity to SEQ ID NO:3. In some embodiments, the engineered immune cell is a CD8+ T cell comprising a CD7 PEBL encoded by the bicistronic construct comprising a nucleic acid sequence of the CD7 PEBL having at least 90% sequence identity to SEQ ID NO:3. In some embodiments, the engineered immune cell is a CD3+ T cell comprising a CD7 PEBL encoded by the bicistronic construct comprising a nucleic acid sequence of the CD7 PEBL having at least 90% sequence identity to SEQ ID NO:3. Also, provided herein is a population comprising such cells.

Chimeric Antigen Receptors that Bind CD7

In some embodiments, the CAR of the present invention comprises intracellular signaling domains of 4-1BB and CD3ζ, and an antigen binding domain (e.g., a single chain variable fragment or scFv) that specifically binds CD7. The CD7 CAR of the present invention is sometimes referred to herein as “anti-CD7-41BB-CD3C”. In some embodiments, the CAR also includes a CD8a hinge domain and transmembrane domain, such as but not limited the amino acid sequence of SEQ ID NO:84.

As those skilled in the art would appreciate, in certain embodiments, any of the amino acid sequences of the various components disclosed herein (e.g., scFv, intracellular signaling domain, linker, and combinations thereof) can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the specific corresponding sequences disclosed herein. For example, in certain embodiments, the intracellular signaling domain 4-1BB can have at least 90% sequence identity, at least 91% sequence identity, at least 92% sequence identity, at least 93% sequence identity, at least 94% sequence identity, at least 95% sequence identity, at least 95% sequence identity, at least 96% sequence identity, at least 97% sequence identity, at least 98% sequence identity, at least 99% sequence identity, or 100% sequence identity to SEQ ID NO:85, as long as it possesses the desired function. In certain embodiments, the intracellular signaling domain of 4-1BB comprises the amino acid sequence set forth in SEQ ID NO:85.

As another example, in certain embodiments, the intracellular signaling domain 4-1BB can be replaced by another intracellular signaling domain from a co-stimulatory molecule such as CD28, OX40, ICOS, CD27, GITR, HVEM, TIM1, LFA1, or CD2. In some embodiments, the intracellular signaling domain of the CAR can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the intracellular signaling domain of CD28, OX40, ICOS, CD27, GITR, HVEM, TIM1, LFA1, or CD2.

As another example, in certain instances, the intracellular signaling domain of 4-1BB can also include another intracellular signaling domain (or a portion thereof) from a co-stimulatory molecule such as CD28, OX40, ICOS, CD27, GITR, HVEM, TIM1, LFA1, or CD2. In some embodiments, the additional intracellular signaling domain can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the intracellular signaling domain of CD28, OX40, ICOS, CD27, GITR, HVEM, TIM1, LFA1, or CD2. In other embodiments, the additional intracellular signaling domain comprises at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one or more intracellular signaling domain fragment(s) of CD28, OX40, ICOS, CD27, GITR, HVEM, TIM1, LFA1, or CD2.

As another example, in certain embodiments, the intracellular signaling domain CD3ζ can have at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to SEQ ID NO:86, as long as it possesses the desired function. In certain embodiments, the intracellular signaling domain of CD3ζ comprises the amino acid sequence set forth in SEQ ID NO:86.

In some instances, the intracellular signaling domain comprises an immunoreceptor tyrosine-based activation motif (ITAM) or a portion thereof, as long as it possesses the desired function. The intracellular signaling domain of the CAR can include a sequence having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to an ITAM. In certain embodiments, the intracellular signaling domain can have at least 95% sequence identity, at least 96% sequence identity, at least 97% sequence identity, at least 98% sequence identity, at least 99% sequence identity, or 100% sequence identity to FcεRIγ, CD4, CD7, CD8, CD28, OX40 or H2-Kb, as long as it possesses the desired function.

In certain embodiments, the anti-CD7 CAR further comprises a hinge domain and/or a transmembrane domain. Hinge and transmembrane domains suitable for use in the present invention are known in the art, and provided in, e.g., publication WO2016/126213, incorporated by reference in its entirety. In some embodiments, the hinge and transmembrane domains of the anti-CD7 CAR includes a signaling domain (e.g., hinge and transmembrane domains) from CD80, 4-1BB, CD28, CD34, CD4, FcεRIγ, CD16, OX40, CD3ζ, CD3ε, CD3γ, CD3δ, TCRα, CD32, CD64, VEGFR2, FAS, FGFR2B, or another transmembrane protein.

In certain embodiments, the anti-CD7 CAR further comprises a CD8c signal peptide. A schematic of the anti-CD7 CAR comprising the embodiments described herein is shown in FIG. 17 of US 2018/0179280.

In some embodiments, the chimeric antigen receptor (CAR) can bind to a molecule that is expressed on the surface of a cell including, but not limited to members of the CD1 family of glycoproteins, CD2, CD3, CD4, CD5, CD7, CD8, CD25, CD28, CD30, CD38, CD45, CD45RA, CD45RO, CD52, CD56, CD57, CD99, CD127, and CD137.

In certain embodiments, an isolated polynucleotide of the present invention comprises a nucleic acid sequence that encodes a CAR according to Table 7. In some embodiments, the polynucleotide comprises a nucleic acid sequence that encodes a component of the CAR according to Table 7.

TABLE 7
Amino acid sequence information for select
components of a CD7 CAR

| | SEQ ID | |
|---|---|---|
| Component | NO | Amino Acid Sequence |
| CD8α hinge and | SEQ ID | TTTPAPRPPTPAPTIASQPLSLRPEA |
| transmembrane | NO: 84 | CRPAAGGAVHTRGLDFACDIYIWAPL |
| domain | | AGTCGVLLLSLVITLY |
| Intracellular | SEQ ID | KRGRKKLLYIFKQPFMRPVQTTQEED |
| signaling | NO: 85 | GCSCRFPEEEEGGCEL |
| domain of | | |
| 4-1BB | | |
| Intracellular | SEQ ID | RVKFSRSADAPAYQQGQNQLYNELNL |
| signaling | NO: 86 | GRREEYDVLDKRRGRDPEMGGKPRRK |
| domain CD3ζ | | NPQEGLYNELQKDKMAEAYSEIGMKG |
| | | ERRRGKGHDGLYQGLSTATKDTYDAL |
| | | HMQALPPR |

In some embodiments, the CD7 CAR comprises a CD7 antigen binding domain, a 4-1BB intracellular signaling domain, a CD3ζ intracellular signaling domain, and CD8 hinge and transmembrane domain. In some embodiments, the CD7 antigen binding domain comprises a VH domain and a VL domain, and a VH-VL linker, such as but not limited to a (G₄S)ₙ linker where n can range from 1 to 6, e.g., 1, 2, 3, 4, 5, or 6. In some embodiments, the CD7 CAR comprises from N-terminus to C-terminus: a CD8 signal peptide, a CD7 antigen binding domain, a CD8 hinge and transmembrane domain, a 4-1BB intracellular signaling domain, and a CD3ζ intracellular signaling domain.

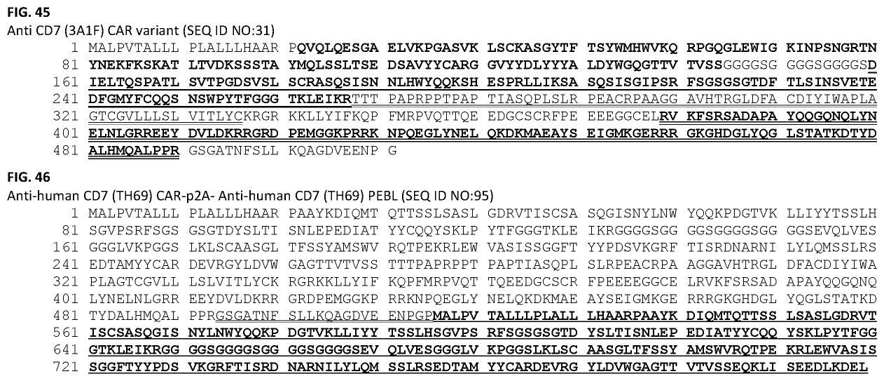

In some embodiments, the CD7 CAR encoded by the bicistronic vector described herein comprises the amino acid sequence of SEQ ID NO:28. In some embodiments, the CD7 CAR encoded by the bicistronic vector described herein comprises the amino acid sequence of SEQ ID NO:29. In some embodiments, the CD7 CAR encoded by the bicistronic vector described herein comprises the amino acid sequence of SEQ ID NO:30. In some embodiments, the CD7 CAR encoded by the bicistronic vector described herein comprises the amino acid sequence of SEQ ID NO:31. Exemplary embodiments of CD7 CARs of the present invention are depicted in FIGS. 42-45.

In some embodiments, an engineered immune cell of the present invention comprises a CD7 CAR encoded by a bicistronic vector such that the CD7 CAR comprises the sequence of SEQ ID NO:28 and additional amino acid residues at the N-terminus produced by cleavage of the 2A self-cleaving peptide, or the CD7 CAR comprises the sequence of SEQ ID NO:29. In some embodiments, the engineered immune cell is a CD4+ T cell comprising a CD7 CAR encoded by a bicistronic vector such that the CD7 CAR comprises the sequence of SEQ ID NO:28 and additional amino acid residues at the N-terminus produced by cleavage of the 2A self-cleaving peptide, or the CD7 CAR comprises the sequence of SEQ ID NO:29. In some embodiments, the engineered immune cell is a CD8+ T cell comprising a CD7 CAR encoded by a bicistronic vector such that the CD7 CAR comprises the sequence of SEQ ID NO:28 and additional amino acid residues at the N-terminus produced by cleavage of the 2A self-cleaving peptide, or the CD7 CAR comprises the sequence of SEQ ID NO:29. In some embodiments, the engineered immune cell is a CD3+ T cell comprising a CD7 CAR encoded by a bicistronic vector such that the CD7 CAR comprises the sequence of SEQ ID NO:28 and additional amino acid residues at the N-terminus produced by cleavage of the 2A self-cleaving peptide, or the CD7 CAR comprises the sequence of SEQ ID NO:29. Also, provided herein are populations comprising such cells.

In some embodiments, an engineered immune cell of the present invention comprises a CD7 CAR encoded by a bicistronic vector such that the CD7 CAR comprises the sequence of SEQ ID NO:30 and additional amino acid residues at the N-terminus produced by cleavage of the 2A self-cleaving peptide, or the CD7 CAR comprises the sequence of SEQ ID NO:31. In some embodiments, the engineered immune cell is a CD4+ T cell comprising a CD7 CAR encoded by a bicistronic vector such that the CD7 CAR comprises the sequence of SEQ ID NO:30 and additional amino acid residues at the N-terminus produced by cleavage of the 2A self-cleaving peptide, or the CD7 CAR comprises the sequence of SEQ ID NO:31. In some embodiments, the engineered immune cell is a CD8+ T cell comprising a CD7 CAR encoded by a bicistronic vector such that the CD7 CAR comprises the sequence of SEQ ID NO:30 and additional amino acid residues at the N-terminus produced by cleavage of the 2A self-cleaving peptide, or the CD7 CAR comprises the sequence of SEQ ID NO:31. In some embodiments, the engineered immune cell is a CD3+ T cell comprising a CD7 CAR encoded by a bicistronic vector such that the CD7 CAR comprises the sequence of SEQ ID NO:30 and additional amino acid residues at the N-terminus produced by cleavage of the 2A self-cleaving peptide, or the CD7 CAR comprises the sequence of SEQ ID NO:31. Also, provided herein are populations of such cells.

In some embodiments, the CD7 CAR encoded by the dual promoter vector described herein comprises the amino acid sequence of SEQ ID NO:28. In some embodiments, an engineered immune cell of the present invention comprises a CD7 CAR encoded by a dual promoter vector such that the CD7 CAR comprises the sequence of SEQ ID NO:28. In some embodiments, the engineered immune cell is a CD4+ T cell comprising a CD7 CAR encoded by a dual promoter vector such that the CD7 CAR comprises the sequence of SEQ ID NO:28. In some embodiments, the engineered immune cell is a CD8+ T cell comprising a CD7 CAR encoded by a dual promoter vector such that the CD7 CAR comprises the sequence of SEQ ID NO:28. In some embodiments, the engineered immune cell is a CD3+ T cell comprising a CD7 CAR encoded by a dual promoter vector such that the CD7 CAR comprises the sequence of SEQ ID NO:28. In some embodiments, the CD7 CAR encoded by the bicistronic vector described herein comprises the amino acid sequence of SEQ ID NO:30. In some embodiments, an engineered immune cell of the present invention comprises a CD7 CAR encoded by a dual promoter vector such that the CD7 CAR comprises the sequence of SEQ ID NO:30. In some embodiments, the engineered immune cell is a CD4+ T cell comprising a CD7 CAR encoded by a dual promoter vector such that the CD7 CAR comprises the sequence of SEQ ID NO:30. In some embodiments, the engineered immune cell is a CD8+ T cell comprising a CD7 CAR encoded by a dual promoter vector such that the CD7 CAR comprises the sequence of SEQ TD NO:30. In some embodiments, the engineered immune cell is a CD3+ T cell comprising a CD7 CAR encoded by a dual promoter vector such that the CD7 CAR comprises the sequence of SEQ ID NO:30. Also, provided herein are populations of such cells.

In certain embodiments, an isolated polynucleotide of a CD7 CAR of the present invention comprises one or more nucleic acid sequences of Table 8. In some embodiments, the nucleic acid sequence comprises a sequence encoding one or more components of the CAR as set forth in Table 8.

TABLE 8
Nucleic acid sequence information for select
components of a CD7 CAR

| | SEQ ID | |
|---|---|---|
| Component | NO | Nucleic Acid Sequence |
| CD8α hinge | SEQ ID | ACCACTACACCTGCACCAAGGCCTCCCAC |
| and trans- | NO: 96 | ACCCGCTCCCACTATCGCTTCCCAGCCAC |
| membrane | | GTTCCCTGAGGCCCGAGGCCTGCAGGCCA |
| domain | | GCAGCTGGCGGAGCCGTGCATACTAGGGG |
| | | GCTGGACTTCGCTTGCGACATCTACATCT |
| | | GGGCCCCACTGGCAGGGACATGCGGAGTC |
| | | CTGCTGCTGTCCCTGGTCATCACACTGTA |
| | | CTGC |
| Intracel- | SEQ ID | AAGCGGGGGCGCAAAAAACTGCTGTATAT |
| lular | NO: 97 | CTTTAAGCAGCCTTTCATGAGACCAGTGC |
| signaling | | AGACAACCCAGGAGGAAGATGGGTGCTCA |
| domain of | | TGCCGGTTTCCCGAGGAGGAGGAAGGCGG |
| 4-1BB | | CTGCGAGCTG |
| Intracel- | SEQ ID | GGGTGAAGTTTTCCCGCTCAGCAGATGCT |
| lular | NO: 98 | CCTGCCTACCAGCAGGGCCAGAACCAGCT |
| signaling | | GTATAATGAGCTGAACCTGGGCAGACGCG |
| domain of | | AAGAGTATGATGTGCTGGACAAAAGGCGG |
| CD3ζ | | GGAAGAGACCCCGAAATGGGAGGGAAGCC |
| | | AAGGCGGAAAAACCCCCAGGAGGGCCTGT |
| | | ACAATGAGCTGCAGAAGGACAAAATGGCA |
| | | GAGGCTTACAGTGAGATTGGGATGAAGGG |
| | | AGAGAGACGGAGGGGAAAAGGGCACGATG |
| | | GCCTGTACCAGGGGCTGAGCACAGCAACC |
| | | AAAGATACTTATGACGCACTGCACATGCA |
| | | GGCACTGCCACCCAGA |

In some embodiments, the polynucleotide encoding the CD7 CAR comprises a nucleic acid sequence for an antigen binding domain that binds CD7, a nucleic acid sequence for a CD8a hinge and transmembrane domain, a nucleic acid sequence for an intracellular signaling domain of 4-1BB, and intracellular signaling domain of CD3ζ. In certain embodiments, the polynucleotide also includes a nucleic acid sequence for a CD8 signal peptide.

In certain embodiments, the antigen binding domain is a anti-CD7 scFv. In some embodiments, the VH sequence of the scFv comprises a nucleic acid sequence having at least 90% sequence identity (e.g., 90%, 91%, 92%, 93%, 94%, 95%, 96%, 97%, 98%, 99%, or more sequence identity) to the sequence of SEQ ID NO:38 and the VL sequence comprises a nucleic acid sequence having at least 90% sequence identity (e.g., 90%, 91%, 92%, 93%, 94%, 95%, 96%, 97%, 98%, 99%, or more sequence identity) to the sequence of SEQ ID NO:39.

In some embodiments, the VH sequence of the scFv comprises a nucleic acid sequence having at least 90% sequence identity (e.g., 90%, 91%, 92%, 93%, 94%, 95%, 96%, 97%, 98%, 99%, or more sequence identity) to the sequence of SEQ ID NO:40 and the VL sequence comprises a nucleic acid sequence having at least 90% sequence identity (e.g., 90%, 91%, 92%, 93%, 94%, 95%, 96%, 97%, 98%, 99%, or more sequence identity) to the sequence of SEQ ID NO:41. In some embodiments, the VH sequence of the scFv comprises a nucleic acid sequence having at least 90% sequence identity (e.g., 90%, 91%, 92%, 93%, 94%, 95%, 96%, 97%, 98%, 99%, or more sequence identity) to the sequence of SEQ ID NO:42 and the VL sequence comprises a nucleic acid sequence having at least 90% sequence identity (e.g., 90%, 91%, 92%, 93%, 94%, 95%, 96%, 97%, 98%, 99%, or more sequence identity) to the sequence of SEQ ID NO:43.

In some embodiments, the polynucleotide encoding the CD7 CAR comprises from the 5′ end to the 3′ end: a nucleic acid sequence for an antigen binding domain that binds CD7, SEQ ID NO:96, SEQ ID NO:97, and SEQ ID NO:98. In some embodiments, the polynucleotide encoding the CD7 CAR comprises from the 5′ end to the 3′ end: SEQ ID NO:81, a nucleic acid sequence for an antigen binding domain that binds CD7, SEQ ID NO:96, SEQ ID NO:97, and SEQ ID NO:98.

In some embodiments, the CD7 CAR comprises a nucleic acid sequence having at least 85% (e.g., 85%, 86%, 87%, 88%, 89%, 90%, or more) sequence identity to SEQ ID NO:4 and binds to CD7. In some embodiments, the CD7 CAR comprises a nucleic acid sequence having at least 90% (e.g., 90%, 91%, 92%, 94%, 94%, 95%, 96%, 97%, 98%, 99%, or more) sequence identity to SEQ ID NO:3 and binds to CD7. In some embodiments, the CD7 PEBL comprises a nucleic acid sequence having at least 90% (e.g., 90%, 91%, 92%, 94%, 94%, 95%, 96%, 97%, 98%, 99%, or more) sequence identity to SEQ ID NO:4.

In some embodiments, the CD7 CAR comprises a nucleic acid sequence having at least 85% (e.g., 85%, 86%, 87%, 88%, 89%, 90%, or more) sequence identity to SEQ ID NO:5 and binds to CD7. In some embodiments, the CD7 CAR comprises a nucleic acid sequence having at least 90% (e.g., 90%, 91%, 92%, 94%, 94%, 95%, 96%, 97%, 98%, 99%, or more) sequence identity to SEQ ID NO:3 and binds to CD7. In some embodiments, the CD7 PEBL comprises a nucleic acid sequence having at least 90% (e.g., 90%, 91%, 92%, 94%, 94%, 95%, 96%, 97%, 98%, 99%, or more) sequence identity to SEQ ID NO:5.

In some embodiments, an engineered immune cell of the present invention comprises a CD7 CAR encoded by a bicistronic construct or a dual promoter construct comprising a nucleic acid sequence of the CD7 CAR having at least 90% (e.g., 90%, 91%, 92%, 94%, 94%, 95%, 96%, 97%, 98%, 99%, or more) sequence identity to SEQ ID NO:4. In some embodiments, the engineered immune cell is a CD4+ T cell comprising a CD7 CAR encoded by the bicistronic vector construct or a dual promoter construct comprising a nucleic acid sequence of the CD7 PEBL having at least 90% sequence identity to SEQ ID NO:4. In some embodiments, the engineered immune cell is a CD8+ T cell comprising a CD7 PEBL encoded by the bicistronic construct comprising a nucleic acid sequence of the CD7 PEBL having at least 90% sequence identity to SEQ ID NO:4. In some embodiments, the engineered immune cell is a CD3+ T cell comprising a CD7 PEBL encoded by the bicistronic construct comprising a nucleic acid sequence of the CD7 PEBL having at least 90% sequence identity to SEQ ID NO:4. Also, provided herein is a population comprising such cells.

In some embodiments, an engineered immune cell of the present invention comprises a CD7 CAR encoded by a bicistronic construct or a dual promoter construct comprising a nucleic acid sequence of the CD7 CAR having at least 90% (e.g., 90%, 91%, 92%, 94%, 94%, 95%, 96%, 97%, 98%, 99%, or more) sequence identity to SEQ ID NO:5. In some embodiments, the engineered immune cell is a CD4+ T cell comprising a CD7 CAR encoded by the bicistronic vector construct comprising a nucleic acid sequence of the CD7 PEBL having at least 90% sequence identity to SEQ ID NO:5. In some embodiments, the engineered immune cell is a CD8+ T cell comprising a CD7 PEBL encoded by the bicistronic construct comprising a nucleic acid sequence of the CD7 PEBL having at least 90% sequence identity to SEQ ID NO:5. In some embodiments, the engineered immune cell is a CD3+ T cell comprising a CD7 PEBL encoded by the bicistronic construct comprising a nucleic acid sequence of the CD7 PEBL having at least 90% sequence identity to SEQ ID NO:5. Also, provided herein is a population comprising such cells.

### Engineered Immune Cells Expressing Bicistronic Vectors

In certain embodiments, provided is an engineered immune cell comprising a bicistronic construct comprising: (i) a polynucleotide encoding a chimeric antigen receptor (CAR), wherein the CAR comprises intracellular signaling domains of 4-1BB and CD3ζ, and an antigen binding domain that specifically binds CD7; (ii) a polynucleotide encoding a target-binding molecule linked to a localizing domain, wherein the target-binding molecule is an antigen binding domain that binds CD7, and the localizing domain comprises an endoplasmic reticulum retention sequence; and (iii) a nucleic acid sequence encoding a 2A self-cleaving peptide or an IRES sequence, as exemplified herein.

In certain embodiments, the antigen binding domain that binds CD7 in the context of the CAR, as well as in the context of the antigen binding domain against CD7 comprises: a VH sequence set forth in SEQ ID NO:32 and a VL sequence set forth in SEQ ID NO:33; a VH sequence set forth in SEQ ID NO:34 and a VL sequence set forth in SEQ ID NO:35; or a VH sequence set forth in SEQ ID NO:36 and a VL sequence set forth in SEQ ID NO:37. As described herein, in certain embodiments, the antigen binding domain comprises a VH and a VL having sequence that each comprise at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the VH and VL sequences set forth in SEQ ID NOS:32 and 33, respectively; SEQ ID SEQ ID NOS:34 and 35, respectively; or SEQ ID NOS:36 and 37, respectively. In certain embodiments, the antigen binding domain that binds CD7 in the context of the CAR can be different from the antibody that binds CD7 in the context of the target-binding molecule (the protein expression blocker or PEBL), as described herein.

In some embodiments, the engineered immune cell comprising a bicistronic construct comprising a nucleic acid construct comprising from the 5′ end to 3′ end: a polynucleotide encoding a target-binding molecule linked to a localizing domain wherein the target-binding molecule binds CD7 (e.g., a CD7 PEBL), an IRES sequence, and a polynucleotide encoding a chimeric antigen receptor against CD7 (e.g., a CD7 CAR). In some instances, the engineered immune cell comprises a nucleic acid construct comprising SEQ ID NO:11. In some embodiments, provided herein is an engineered CD4+ T cell or a population thereof comprising a nucleic acid construct comprising SEQ ID NO:11. In other embodiments, provided herein is an engineered CD8+ T cell or a population thereof comprising a nucleic acid construct comprising SEQ ID NO:11. In some embodiments, provided herein is an engineered CD3+ T cell or a population thereof comprising a nucleic acid construct comprising SEQ ID NO:11.

In some embodiments, the engineered immune cell comprising a bicistronic construct comprising a nucleic acid construct comprising from the 5′ end to 3′ end: a polynucleotide encoding a chimeric antigen receptor against CD7, a IRES sequence, and a polynucleotide encoding a target-binding molecule linked to a localizing domain wherein the target-binding molecule binds CD7 (e.g., a PEBL against CD7). In some instances, the engineered immune cell comprises a nucleic acid construct comprising SEQ ID NO:12 or the sequence depicted in FIG. 26. In one embodiment, provided herein is an engineered CD4+ T cell or a population thereof comprising a nucleic acid construct comprising SEQ ID NO:12 or the sequence depicted in FIG. 26. In some embodiments, provided herein is an engineered CD8+ T cell or a population thereof comprising a nucleic acid construct comprising SEQ ID NO:12 or the sequence depicted in FIG. 26. In some embodiments, provided herein is an engineered CD3+ T cell or a population thereof comprising a nucleic acid construct comprising SEQ ID NO:12 or the sequence depicted in FIG. 26.

In some embodiments, the engineered immune cell comprising a bicistronic construct comprising a nucleic acid construct comprising from the 5′ end to 3′ end: a polynucleotide encoding a chimeric antigen receptor against CD7 (e.g., a CD7 CAR), a nucleic acid sequence encoding a 2A self-cleaving peptide, and a polynucleotide encoding a target-binding molecule linked to a localizing domain wherein the target-binding molecule binds CD7 (e.g., a CD7 PEBL). In some instances, the engineered immune cell comprises a nucleic acid construct comprising SEQ ID NO:13 or the sequence depicted in FIG. 27. In one embodiment, provided herein is an engineered CD4+ T cell or a population thereof comprising a nucleic acid construct comprising SEQ ID NO:13 or the sequence depicted in FIG. 27. In some embodiments, provided herein is an engineered CD8+ T cell or a population thereof comprising a nucleic acid construct comprising SEQ ID NO:13 or the sequence depicted in FIG. 27. In some embodiments, provided herein is an engineered CD3+ T cell or a population thereof comprising a nucleic acid construct comprising SEQ ID NO:13 or the sequence depicted in FIG. 27.

In some embodiments, the engineered immune cell comprising a bicistronic construct comprising a nucleic acid construct comprising from the 5′ end to 3′ end: a polynucleotide encoding a target-binding molecule linked to a localizing domain wherein the target-binding molecule binds CD7, a nucleic acid sequence encoding a 2A self-cleaving peptide, and a polynucleotide encoding a chimeric antigen receptor against CD7.

In some embodiments, the engineered immune cell comprising a bicistronic construct comprising a nucleic acid construct comprising from the 5′ end to 3′ end: a promoter, a polynucleotide encoding a chimeric antigen receptor against CD7 (e.g., a CD7 CAR), a nucleic acid sequence encoding a 2A self-cleaving peptide, and a polynucleotide encoding a target-binding molecule linked to a localizing domain wherein the target-binding molecule binds CD7 (e.g., a CD7 PEBL). In some instances, the engineered immune cell comprises a nucleic acid construct comprising at least 85% sequence identity to any one of the nucleic acid sequences of SEQ ID NOS:14-16. In some instances, the engineered immune cell comprises a nucleic acid construct comprising any one of the nucleic acid sequences of SEQ ID NOS:14-16. In some embodiments, the engineered immune cell comprises a nucleic acid construct comprising at least 85% (e.g., 85%, 86%, 87%, 88%, 89%, 90%, 91%, 92%, 94%, 94%, 95%, 96%, 97%, 98%, 99%, or more) sequence identity to SEQ ID NO:14. In some embodiments, the engineered immune cell comprises a nucleic acid construct comprising at least 85% (e.g., 85%, 86%, 87%, 88%, 89%, 90%, 91%, 92%, 94%, 94%, 95%, 96%, 97%, 98%, 99%, or more) sequence identity to SEQ ID NO:15. In some embodiments, the engineered immune cell comprises a nucleic acid construct comprising at least 85% (e.g., 85%, 86%, 87%, 88%, 89%, 90%, 91%, 92%, 94%, 94%, 95%, 96%, 97%, 98%, 99%, or more) sequence identity to SEQ ID NO:16.

In some embodiments, the engineered immune cell comprising a bicistronic construct comprising a nucleic acid construct comprising from the 5′ end to 3′ end: a promoter, a polynucleotide encoding a chimeric antigen receptor against CD7, a nucleic acid sequence encoding a 2A self-cleaving peptide, and a polynucleotide encoding a target-binding molecule linked to a localizing domain wherein the target-binding molecule binds CD7. In some instances, the promoter is selected from a MSCV promoter, PGK promoter, EF1α promoter, and EFS promoter. In some instances, the engineered immune cell comprises a polynucleotide comprising SEQ ID NO:14 or the sequence as depicted in FIGS. 28A-28B. In one embodiment, provided herein is an engineered CD4+ T cell or a population thereof comprising a nucleic acid construct comprising SEQ ID NO:14 or the sequence depicted in FIGS. 28A-B. In some embodiments, provided herein is an engineered CD8+ T cell or a population thereof comprising a nucleic acid construct comprising SEQ ID NO:14 or the sequence depicted in FIGS. 28A-B. In some embodiments, provided herein is an engineered CD3+ T cell or a population thereof comprising a nucleic acid construct comprising SEQ ID NO:14 or the sequence depicted in FIGS. 28A-B.

In some instances, the engineered immune cell comprises a polynucleotide comprising SEQ ID NO:15. In one embodiment, provided herein is an engineered CD4+ T cell or a population thereof comprising a nucleic acid construct comprising SEQ ID NO:15 or the sequence depicted in FIGS. 29A-B. In some embodiments, provided herein is an engineered CD8+ T cell or a population thereof comprising a nucleic acid construct comprising SEQ ID NO:15 or the sequence depicted in FIGS. 29A-B. In one embodiment, provided herein is an engineered CD4+ T cell or a population thereof comprising a nucleic acid construct comprising SEQ ID NO:15 or the sequence depicted in FIGS. 29A-B. In some embodiments, provided herein is an engineered CD3+ T cell or a population thereof comprising a nucleic acid construct comprising SEQ ID NO:15 or the sequence depicted in FIGS. 29A-B.

In some instances, the engineered immune cell comprises a polynucleotide comprising SEQ ID NO:16 or the sequence depicted in FIGS. 30A-B. In one embodiment, provided herein is an engineered CD4+ T cell or a population thereof comprising a nucleic acid construct comprising SEQ ID NO:16 or the sequence depicted in FIGS. 30A-B. In some embodiments, provided herein is an engineered CD8+ T cell or a population thereof comprising a nucleic acid construct comprising SEQ ID NO:16 or the sequence depicted in FIGS. 30A-B. In some embodiments, provided herein is an engineered CD3+ T cell or a population thereof comprising a nucleic acid construct comprising SEQ ID NO:16 or the sequence depicted in FIGS. 30A-B.

In some embodiments, the engineered immune cells described herein or a population thereof comprise at least 10% CD7 CAR+/CD7-negative T cells, at least 15% CD7 CAR+/CD7− negative T cells, at least 20% CD7 CAR+/CD7-negative T cells, at least 25% CD7 CAR+/CD7-negative T cells, at least 30% CD7 CAR+/CD7-negative T cells, at least 35% CD7 CAR+/CD7-negative T cells, at least 40% CD7 CAR+/CD7-negative T cells, at least 45% CD7 CAR+/CD7-negative T cells, at least 50% CD7 CAR+/CD7-negative T cells, at least 55% CD7 CAR+/CD7-negative T cells, at least 60% CD7 CAR+/CD7-negative T cells, at least 65% CD7 CAR+/CD7-negative T cells, at least 70% CD7 CAR+/CD7-negative T cells, at least 75% CD7 CAR+/CD7-negative T cells, at least 80% CD7 CAR+/CD7-negative T cells, at least 85% CD7 CAR+/CD7-negative T cells, at least 90% CD7 CAR+/CD7-negative T cells, at least 95% CD7 CAR+/CD7-negative T cells, at least 96% CD7 CAR+/CD7-negative T cells, at least 97% CD7 CAR+/CD7-negative T cells, at least 98% CD7 CAR+/CD7-negative T cells, at least 99% CD7 CAR+/CD7-negative T cells, or 100% CD7 CAR+/CD7-negative T cells. In some embodiments, the engineered immune cells outlined herein include a population of substantially purified CD7 CAR/CD7-negative T cells wherein such cells express any one of the bicistronic constructs described.

### Engineered Immune Cells Expressing Dual Promoter Vectors

In some embodiments, provided is an engineered immune cell comprising a recombinant retroviral vector comprising (a) a first promoter operably linked to a first polynucleotide encoding any of the CARs described herein, and (b) a second promoter operably linked to a second polynucleotide encoding any of the PEBLs described herein. In some embodiments, the engineered immune cell comprises any of the recombinant retroviral vectors described herein containing a promoter driving CAR expression and another promoter driving PEBL expression.

In some embodiments, the engineered immune cell comprises a recombinant retroviral vector comprising a nucleic acid sequence having at least 85% (e.g., 85%, 86%, 87%, 88%, 89%, 90%, 91%, 92%, 94%, 94%, 95%, 96%, 97%, 98%, 99%, or more) sequence identity to SEQ ID NO:17. In some embodiments, the engineered immune cell comprises a recombinant retroviral vector comprising a nucleic acid sequence having at least 85% (e.g., 85%, 86%, 87%, 88%, 89%, 90%, 91%, 92%, 94%, 94%, 95%, 96%, 97%, 98%, 99%, or more) sequence identity to SEQ ID NO:18. In some embodiments, the engineered immune cell comprises a recombinant retroviral vector comprising a nucleic acid sequence having at least 85% (e.g., 85%, 86%, 87%, 88%, 89%, 90%, 91%, 92%, 94%, 94%, 95%, 96%, 97%, 98%, 99%, or more) sequence identity to SEQ ID NO:19. In some embodiments, the engineered immune cell comprises a recombinant retroviral vector comprising a nucleic acid sequence having at least 85% (e.g., 85%, 86%, 87%, 88%, 89%, 90%, 91%, 92%, 94%, 94%, 95%, 96%, 97%, 98%, 99%, or more) sequence identity to SEQ ID NO:20. In some embodiments, the engineered immune cell comprises a recombinant retroviral vector comprising a nucleic acid sequence having at least 85% (e.g., 85%, 86%, 87%, 88%, 89%, 90%, 91%, 92%, 94%, 94%, 95%, 96%, 97%, 98%, 99%, or more) sequence identity to SEQ ID NO:21. In some embodiments, the engineered immune cell comprises a recombinant retroviral vector comprising a nucleic acid sequence having at least 85% (e.g., 85%, 86%, 87%, 88%, 89%, 90%, 91%, 92%, 94%, 94%, 95%, 96%, 97%, 98%, 99%, or more) sequence identity to SEQ ID NO:22. In some embodiments, the engineered immune cell comprises a recombinant retroviral vector comprising a nucleic acid sequence having at least 85% (e.g., 85%, 86%, 87%, 88%, 89%, 90%, 91%, 92%, 94%, 94%, 95%, 96%, 97%, 98%, 99%, or more) sequence identity to SEQ ID NO:23. In some instances, the engineered immune cell is an engineered CD4+ T cell or a population thereof or a population comprising such. In some instances, the engineered immune cell is an engineered CD8+ T cell or a population thereof or a population comprising such.

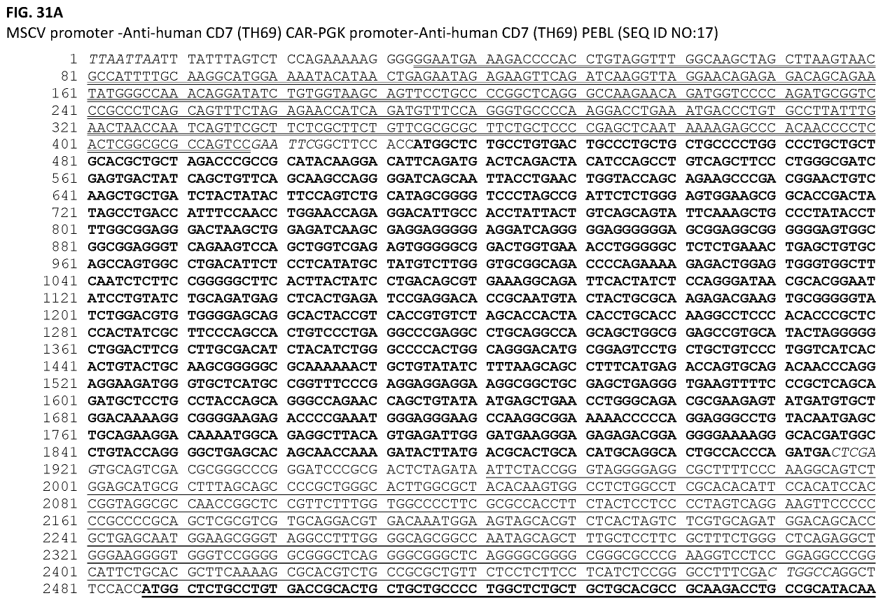

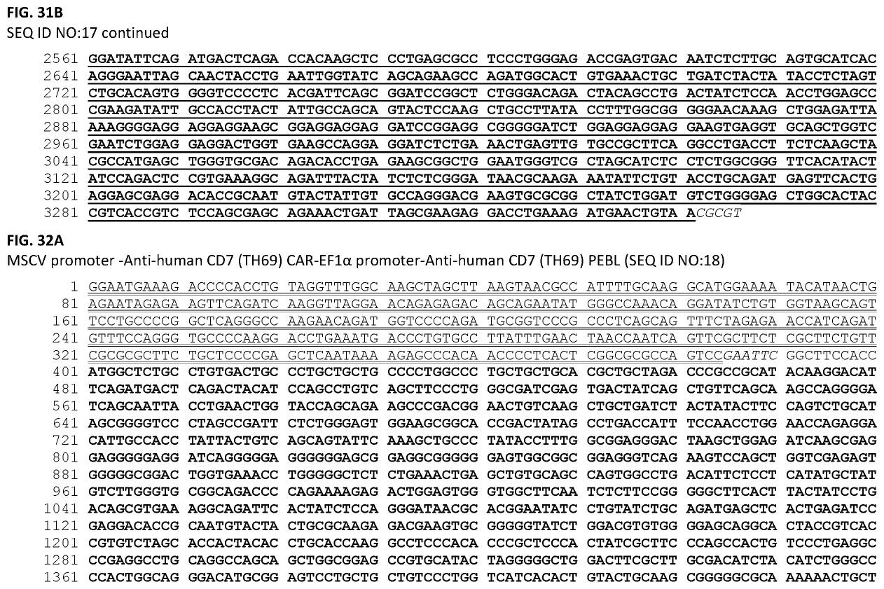

In some instances, the engineered immune cell comprises a polynucleotide comprising SEQ ID NO:17 or the sequence depicted in FIGS. 31A-B. In one embodiment, provided herein is an engineered CD4+ T cell or a population thereof comprising a nucleic acid construct comprising SEQ ID NO:17 or the sequence depicted in FIGS. 31A-B. In some embodiments, provided herein is an engineered CD8+ T cell or a population thereof comprising a nucleic acid construct comprising SEQ ID NO:17 or the sequence depicted in FIGS. 31A-B. In some embodiments, provided herein is an engineered CD3+ T cell or a population thereof comprising a nucleic acid construct comprising SEQ ID NO:17 or the sequence depicted in FIGS. 31A-B.

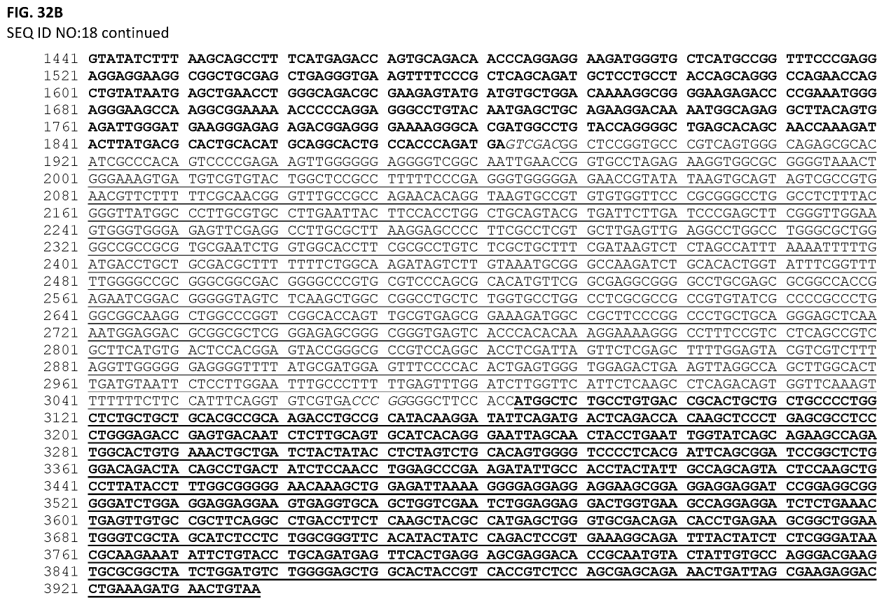

In some instances, the engineered immune cell comprises a polynucleotide comprising SEQ ID NO:18. In one embodiment, provided herein is an engineered CD4+ T cell or a population thereof comprising a nucleic acid construct comprising SEQ ID NO:18 or the sequence depicted in FIGS. 32A-B. In some embodiments, provided herein is an engineered CD8+ T cell or a population thereof comprising a nucleic acid construct comprising SEQ ID NO:18 or the sequence depicted in FIGS. 32A-B. In some embodiments, provided herein is an engineered CD3+ T cell or a population thereof comprising a nucleic acid construct comprising SEQ ID NO:18 or the sequence depicted in FIGS. 32A-B.

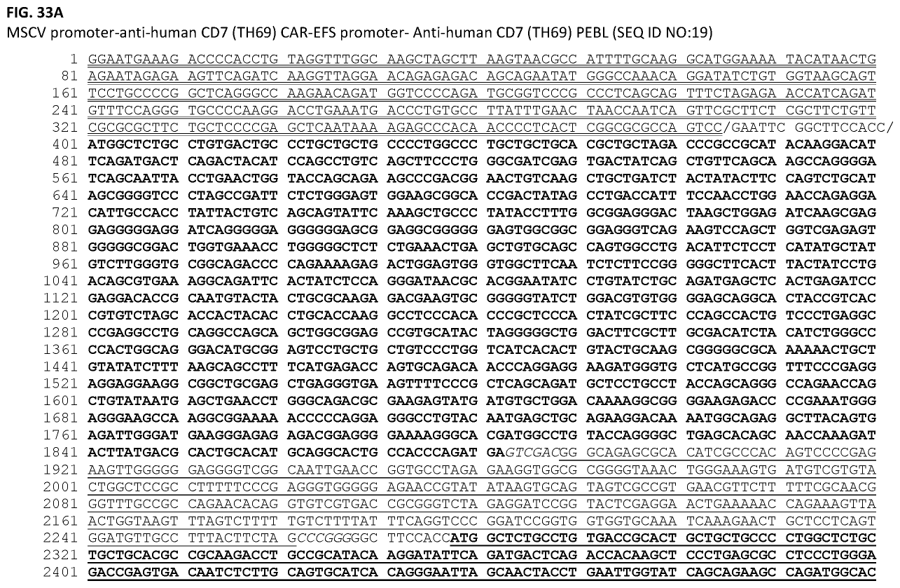

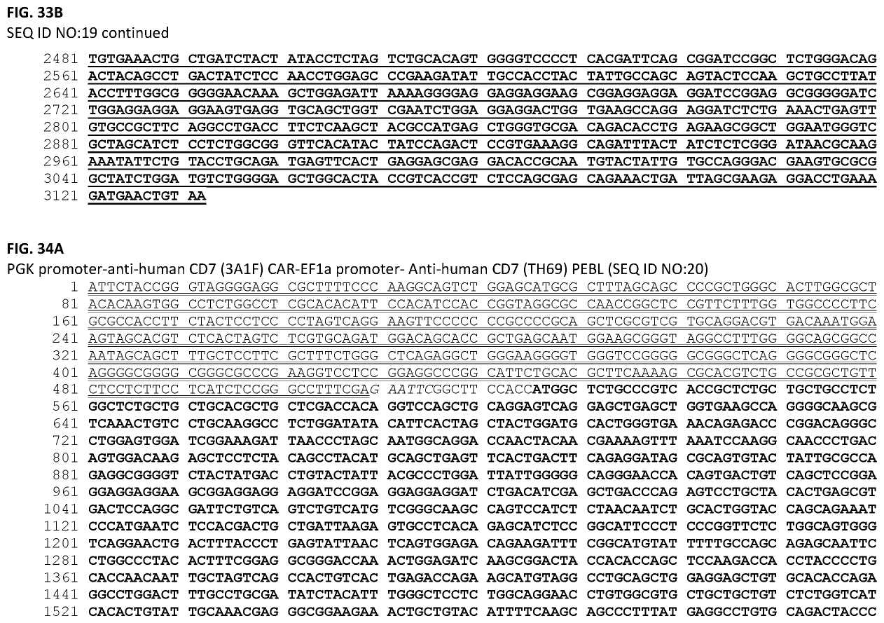

In some instances, the engineered immune cell comprises a polynucleotide comprising SEQ ID NO:19 or the sequence depicted in FIGS. 33A-B. In one embodiment, provided herein is an engineered CD4+ T cell or a population thereof comprising a nucleic acid construct comprising SEQ ID NO:19 or the sequence depicted in FIGS. 33A-B. In some embodiments, provided herein is an engineered CD8+ T cell or a population thereof comprising a nucleic acid construct comprising SEQ ID NO:19 or the sequence depicted in FIGS. 33A-B. In some embodiments, provided herein is an engineered CD3+ T cell or a population thereof comprising a nucleic acid construct comprising SEQ ID NO:19 or the sequence depicted in FIGS. 33A-B.

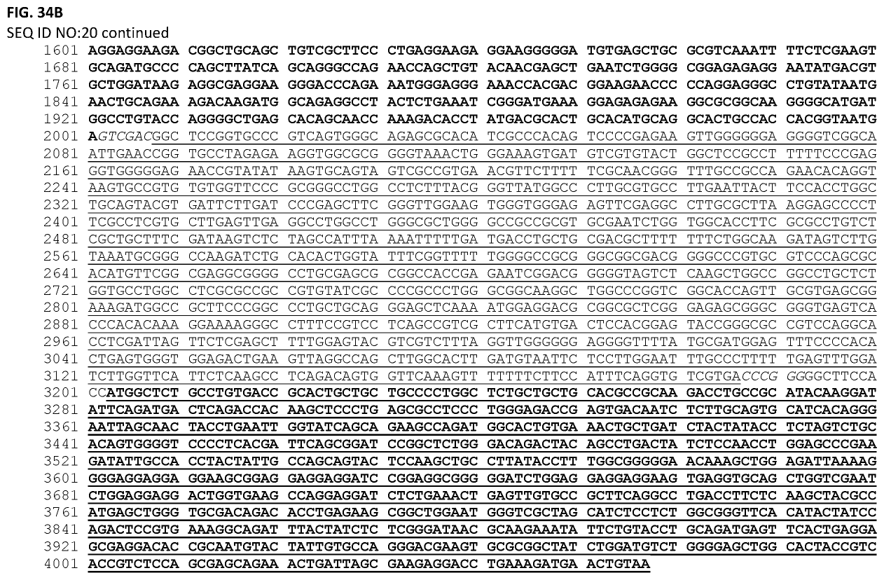

In some instances, the engineered immune cell comprises a polynucleotide comprising SEQ ID NO:20 or the sequence depicted in FIGS. 34A-B. In one embodiment, provided herein is an engineered CD4+ T cell or a population thereof comprising a nucleic acid construct comprising SEQ ID NO:20 or the sequence depicted in FIGS. 34A-B. In some embodiments, provided herein is an engineered CD8+ T cell or a population thereof comprising a nucleic acid construct comprising SEQ ID NO:20 or the sequence depicted in FIGS. 34A-B. In some embodiments, provided herein is an engineered CD3+ T cell or a population thereof comprising a nucleic acid construct comprising SEQ ID NO:20 or the sequence depicted in FIGS. 34A-B.

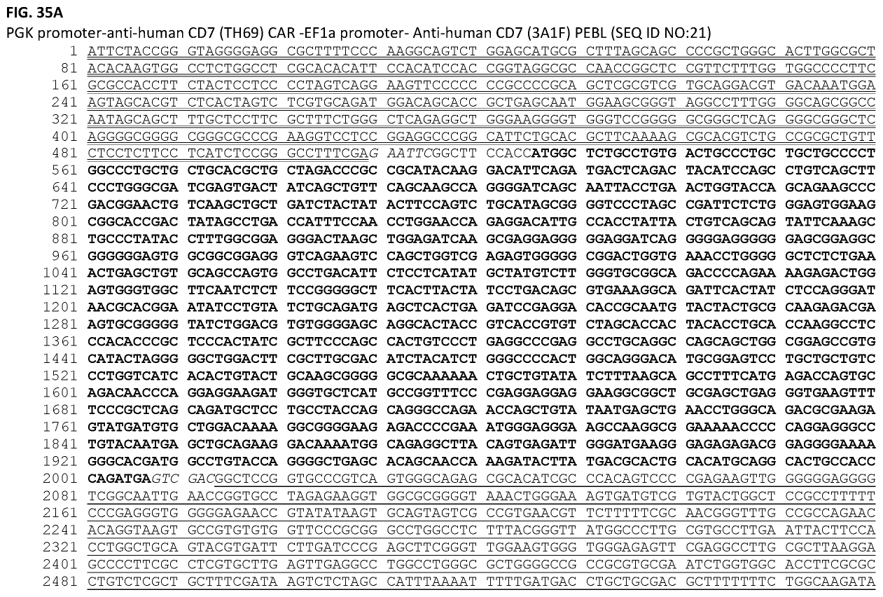

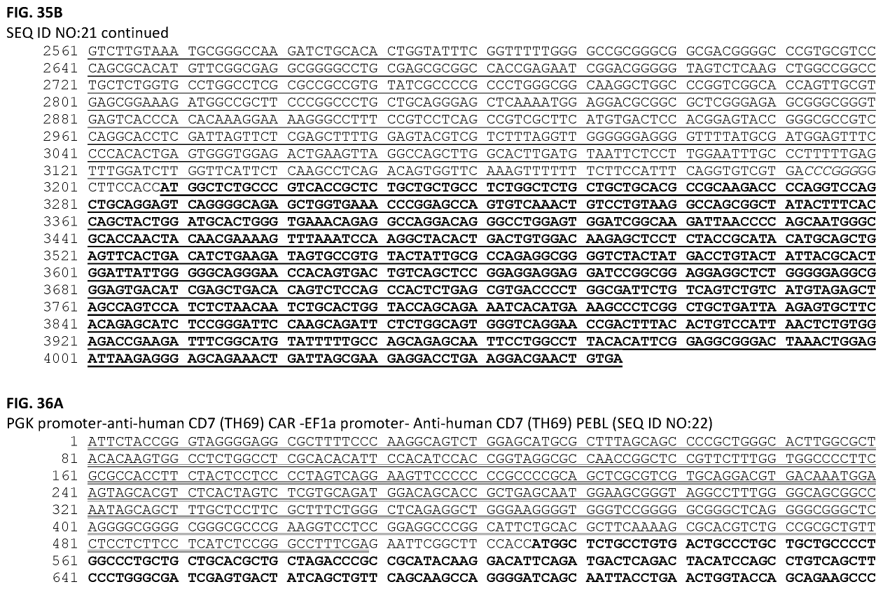

In some instances, the engineered immune cell comprises a polynucleotide comprising SEQ ID NO:21 or the sequence depicted in FIGS. 35A-B. In one embodiment, provided herein is an engineered CD4+ T cell or a population thereof comprising a nucleic acid construct comprising SEQ ID NO:21 or the sequence depicted in FIGS. 35A-B. In some embodiments, provided herein is an engineered CD8+ T cell or a population thereof comprising a nucleic acid construct comprising SEQ ID NO:21 or the sequence depicted in FIGS. 35A-B. In some embodiments, provided herein is an engineered CD3+ T cell or a population thereof comprising a nucleic acid construct comprising SEQ ID NO:21 or the sequence depicted in FIGS. 35A-B.

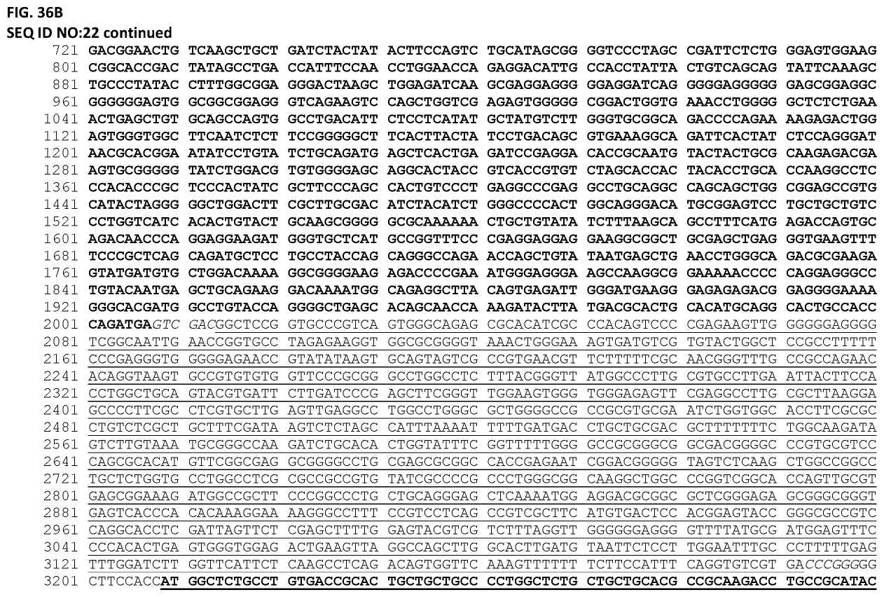

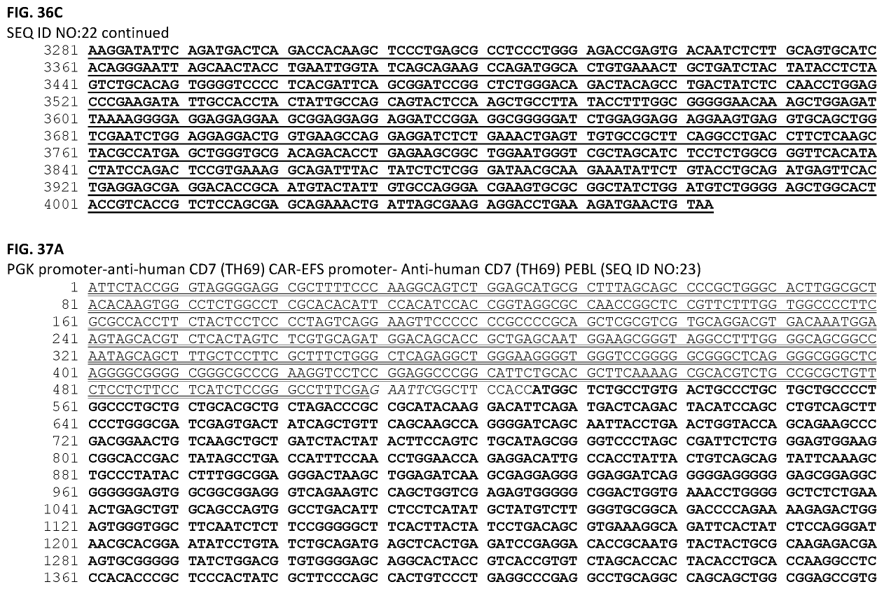

In some instances, the engineered immune cell comprises a polynucleotide comprising SEQ ID NO:22 or the sequence depicted in FIGS. 36A-B. In one embodiment, provided herein is an engineered CD4+ T cell or a population thereof comprising a nucleic acid construct comprising SEQ ID NO:22 or the sequence depicted in FIGS. 36A-B. In some embodiments, provided herein is an engineered CD8+ T cell or a population thereof comprising a nucleic acid construct comprising SEQ ID NO:22 or the sequence depicted in FIGS. 36A-B. In some embodiments, provided herein is an engineered CD3+ T cell or a population thereof comprising a nucleic acid construct comprising SEQ ID NO:22 or the sequence depicted in FIGS. 36A-B.

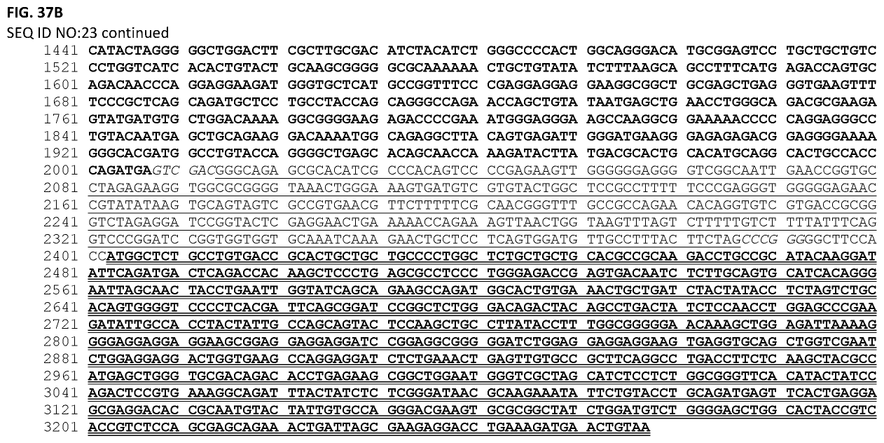

In some instances, the engineered immune cell comprises a polynucleotide comprising SEQ ID NO:23 or the sequence depicted in FIGS. 37A-B. In one embodiment, provided herein is an engineered CD4+ T cell or a population thereof comprising a nucleic acid construct comprising SEQ ID NO:23 or the sequence depicted in FIGS. 37A-B. In some embodiments, provided herein is an engineered CD8+ T cell or a population thereof comprising a nucleic acid construct comprising SEQ ID NO:23 or the sequence depicted in FIGS. 37A-B. In some embodiments, provided herein is an engineered CD3+ T cell or a population thereof comprising a nucleic acid construct comprising SEQ ID NO:23 or the sequence depicted in FIGS. 37A-B.

In some embodiments, the engineered immune cells described herein or a population thereof comprise at least 10% CD7 CAR+/CD7-negative T cells, at least 15% CD7 CAR+/CD7-negative T cells, at least 20% CD7 CAR+/CD7-negative T cells, at least 25% CD7 CAR+/CD7-negative T cells, at least 30% CD7 CAR+/CD7-negative T cells, at least 35% CD7 CAR+/CD7-negative T cells, at least 40% CD7 CAR+/CD7-negative T cells, at least 45% CD7 CAR+/CD7-negative T cells, at least 50% CD7 CAR+/CD7-negative T cells, at least 55% CD7 CAR+/CD7-negative T cells, at least 60% CD7 CAR+/CD7-negative T cells, at least 65% CD7 CAR+/CD7-negative T cells, at least 70% CD7 CAR+/CD7-negative T cells, at least 75% CD7 CAR+/CD7-negative T cells, at least 80% CD7 CAR+/CD7-negative T cells, at least 85% CD7 CAR+/CD7-negative T cells, at least 90% CD7 CAR+/CD7-negative T cells, at least 95% CD7 CAR+/CD7-negative T cells, at least 96% CD7 CAR+/CD7-negative T cells, at least 97% CD7 CAR+/CD7-negative T cells, at least 98% CD7 CAR+/CD7-negative T cells, at least 99% CD7 CAR+/CD7-negative T cells, or 100% CD7 CAR+/CD7-negative T cells. In some embodiments, the engineered immune cells outlined herein include a population of substantially purified CD7 CAR/CD7-negative T cells wherein such cells express any one of the dual promoter constructs described.

CD7 CAR+ Engineered Immune Cells with Reduced Expression of Endogenous CD7

In some embodiments, the engineered immune cells described herein express a CD7 CAR and have reduced or no endogenous CD7 expression compared to a non-engineered immune cell. Such engineered immune cells express a CD7 PEBL that minimizes or eliminates endogenous expression of CD7 on the surface of the immune cell. In some embodiments, reduced expression of CD7 refers to a downregulation or partial downregulation of surface CD7 by the cell. In some cases, reduced expression includes an at least 5% (e.g., at least 5%, 6%, 7%, 8%, 9%, 10%, 11%, 12%, 13%, 14%, 15%, 16%, 17%, 18%, 19%, 20%, 24%, 25%, 28%, 40%, 35%, 40%, 45%, 50%, 55%, 60%, 65%, 70%, 75%, 80%, 85%, 86%, 88%, 90%, 91%, 92%, 93%, 94%, 95%, 96%, 97%, 98%, 99% or 100%) reduction in expression level compared to the expression level of a comparable wild-type or non-engineered cell. In some embodiments, engineered immune cells outlined herein include a population of substantially purified CD7 CAR/CD7-negative T cells.

In some embodiments, the engineered immune cells described herein express a CD7 PEBL having at least 90% sequence identity to SEQ ID NO:24. In some embodiments, the engineered immune cells express a CD7 PEBL having at least 90% sequence identity to SEQ ID NO:25. In some embodiments, the engineered immune cells express a CD7 PEBL having at least 90% sequence identity to SEQ ID NO:26. In some embodiments, the engineered immune cells express a CD7 PEBL having at least 90% sequence identity to SEQ ID NO:27.

In some embodiments, the engineered immune cells described herein express a CD7 CAR having at least 90% sequence identity to SEQ ID NO:28. In some embodiments, the engineered immune cells express a CD7 CAR having at least 90% sequence identity to SEQ ID NO:29. In some embodiments, the engineered immune cells express a CD7 CAR having at least 90% sequence identity to SEQ ID NO:30. In some embodiments, the engineered immune cells express a CD7 CAR having at least 90% sequence identity to SEQ ID NO:31.

In some embodiments, the engineered immune cells described herein express a CD7 PEBL having at least 90% sequence identity to SEQ ID NO:24 and a CD7 CAR having at least 90% sequence identity to SEQ ID NO:28. In some embodiments, the engineered immune cells described herein express a CD7 PEBL having at least 90% sequence identity to SEQ ID NO:24 and a CD7 CAR having at least 90% sequence identity to SEQ ID NO:30.

In some embodiments, the engineered immune cells express a CD7 PEBL having at least 90% sequence identity to SEQ ID NO:25 and express a CD7 CAR having at least 90% sequence identity to SEQ ID NO:29. In some embodiments, the engineered immune cells express a CD7 PEBL having at least 90% sequence identity to SEQ ID NO:25 and express a CD7 CAR having at least 90% sequence identity to SEQ ID NO:31.

In some embodiments, the engineered immune cells express a CD7 PEBL having at least 90% sequence identity to SEQ ID NO:26 and a CD7 CAR having at least 90% sequence identity to SEQ ID NO:28. In some embodiments, the engineered immune cells express a CD7 PEBL having at least 90% sequence identity to SEQ ID NO:26 and a CD7 CAR having at least 90% sequence identity to SEQ ID NO:30.

In some embodiments, the engineered immune cells express a CD7 PEBL having at least 90% sequence identity to SEQ ID NO:27 and express a CD7 CAR having at least 90% sequence identity to SEQ ID NO:29. In some embodiments, the engineered immune cells express a CD7 PEBL having at least 90% sequence identity to SEQ ID NO:27 and express a CD7 CAR having at least 90% sequence identity to SEQ ID NO:31.

In certain embodiments, the engineered immune cell is an engineered T cell, an engineered natural killer (NK) cell, an engineered NK/T cell, an engineered monocyte, an engineered macrophage, or an engineered dendritic cell. In some embodiments, the engineered immune cell is an engineered CD4+ T cell. In some embodiments, the engineered immune cell is an engineered CD8+ T cell. In some embodiments, the engineered immune cell is an engineered CD3+ T cell. Also provided is a population of any one of the engineered cells described herein.

In some embodiments, provided herein is a population of engineered immune cells (e.g., CD3+ T cells, CD4+ T cells, or CD8+ T cells) comprising at least about 50% (e.g., about 50%, 55%, 58%, 60%, 62%, 64%, 66%, 68%, 70%, 71%, 73%, 75%, 77%, 78%, 79%, 80%, 81%, 82%, 83%, 84% 85%, 86%, 87%, 88%, 90%, 91%, 92%, 94%, 94%, 95%, 96%, 97%, 98%, 99%, or more) CD7 CAR-positive, endogenous CD7-negative cells. In some embodiments, provided herein is a population of engineered immune cells comprising at least about 50% (e.g., about 50%, 55%, 58%, 60%, 62%, 64%, 66%, 68%, 70%, 71%, 73%, 75%, 77%, 78%, 79%, 80%, 81%, 82%, 83%, 84% 85%, 86%, 87%, 88%, 90%, 91%, 92%, 94%, 94%, 95%, 96%, 97%, 98%, 99%, or more) CD7 CAR-positive, endogenous CD7-negative CD4+ T cells. In some embodiments, provided herein is a population of engineered immune cells comprising at least about 50% (e.g., about 50%, 55%, 58%, 60%, 62%, 64%, 66%, 68%, 70%, 71%, 73%, 75%, 77%, 78%, 79%, 80%, 81%, 82%, 83%, 84% 85%, 86%, 87%, 88%, 90%, 91%, 92%, 94%, 94%, 95%, 96%, 97%, 98%, 99%, or more) CD7 CAR-positive, endogenous CD7-negative CD8+ T cells. Such a population of cells can be produced from peripheral blood mononuclear cells (PBMC), purified CD4+ T cells, purified CD8+ T cells, or a population comprising purified CD4+ T cells and purified CD8+ T cells.

In some embodiments, the engineered immune cells described herein are cultured to generate a highly pure population of CD7 CAR-T cells that have reduced expression of endogenous CD7. The level of purity can be at least about 75% (e.g., about 75%, 77%, 78%, 79%, 80%, 81%, 82%, 83%, 84% 85%, 86%, 87%, 88%, 90%, 91%, 92%, 94%, 94%, 95%, 96%, 97%, 98%, 99%, or more) CD7 CAR-T cells with no surface expression of CD7. The expression level CD7 can be determined according to standard methods known to those in the art including, but not limited to immunocytochemistry, flow cytometry, and FACS analysis.

In some embodiments, the engineered immune cells of the present invention include CD45RO+ cells. In some embodiments, the engineered immune cells include CCR7-negative cells. In some embodiments, the engineered immune cells include central memory T cells. In some embodiments, the engineered immune cells include effector memory T cells. In some embodiments, the engineered immune cells include effector T cells. In some embodiments, the engineered immune cells include naive T cells.

In some instances, a population of engineered immune cells comprises effector memory T cells, central memory T cells, effector T cells, and naive T cells. In some embodiments, the population of engineered immune cells comprises a higher percentage of effector memory T cells and central memory T cells than effector T cells and naive T cells. In some embodiments, the population of engineered immune cells comprises about 40% or more effector memory T cells.

In some instances, a population of engineered immune cells comprises PD1-negative cells. In some instances, a population of engineered immune cells comprises TIM-1-negative cells. In some embodiments, the population comprises about 60% or more PD1-negative, TIM-1-negative cells. In some embodiments, the population comprises about 4% to about 20% PD1 positive, TIM-1 positive cells.

In some embodiments, the engineered immune cells generate an immune response and secrete interferon-γ. The engineered immune cells induce T-cell mediated cytotoxicity in response to a cancer cell such as a CD7 expressing cancer cell.

In some embodiments, cells described herein comprising a bicistronic expression vector can be used to generate a population of CD7 CAR+/CD7-neg T cells. The CD7 CAR+/CD7-neg T cells can be expanded and enriched over time. The CD7 CAR+/CD7-neg T cells can be generated from cells including, but not limited to, bulk PBMCs, purified T cells comprising CD4+ and CD8+ T cells, and purified CD3+ T cells. The CD7 CAR+/CD7-neg T cells can be used to produce different subsets of T cells including T_{EM} cells, T_{CM} cells, Teff cells, and naïve T cells.

In another aspect, also provided is a method for producing the engineered immune cell (e.g., engineered CD3+ T cell, engineered CD4+ T cell, and engineered CD8+ T cell) having any of the embodiments described herein, the method comprising introducing into an immune cell any of the bicistronic constructs or dual promoter constructs of the present invention. In some embodiments, the engineered immune cells are derived from immune cells obtained from a subject that will receive the engineered immune cells as a therapy. In some embodiments, the engineered immune cells are derived from immune cells obtained from a donor and the resulting engineered immune cells are administered to a subject as a therapy.

In various aspects, also provided is a kit for producing an engineered immune cell described herein. The present kit can be used to produce, e.g., allogeneic or autologous T cells having anti-CD7 CAR-mediated cytotoxic activity. In some embodiments, the kit is useful for producing allogeneic effector T cells having anti-CD7 CAR-mediated cytotoxic activity. In certain embodiments, the kit is useful for producing autologous effector T cells having anti-CD7 CAR-mediated cytotoxic activity.

Accordingly, provided herein is a kit comprising any one of the bicistronic constructs or dual promoter constructs described herein.

In certain embodiments, the bicistronic construct further comprise sequences (e.g., plasmid or vector sequences) that allow, e.g., cloning and/or expression. For example, the nucleotide sequence can be provided as part of a plasmid for ease of cloning into other plasmids and/or vectors (expression vectors or viral expression vectors) for, e.g., transfection, transduction, or electroporation into a cell (e.g., an immune cell).

Typically, the kits are compartmentalized for ease of use and can include one or more containers with reagents. In certain embodiments, all of the kit components are packaged together. Alternatively, one or more individual components of the kit can be provided in a separate package from the other kits components. The kits can also include instructions for using the kit components.

### Administering Engineered Immune Cells

In other aspects, also provided is a method of treating cancer in a subject in need thereof, comprising administering a therapeutic amount of an engineered immune cell having any of the embodiments described herein to the subject, thereby treating cancer in a subject in need thereof.

In certain embodiments, the method comprises administering a therapeutic amount of an engineered immune cell comprising a bicistronic viral construct comprising a polynucleotide comprising a nucleic acid sequence encoding a CAR and a polynucleotide comprising a nucleic acid sequence encoding a PEBL. In various embodiments, the method comprises administering a therapeutic amount of any one of the engineered immune cells described herein comprising a recombinant retroviral vector comprising: (a) a first promoter operably linked to a first polynucleotide encoding a CD7 chimeric antigen receptor (CD7 CAR) as outlined herein; and (b) a second promoter operably linked to a second polynucleotide encoding a CD7 protein expression blocker (CD7 PEBL) as outlined herein.

In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:11 or the sequence depicted in FIG. 25 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:12 or the sequence depicted in FIG. 26 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:13 or the sequence depicted in FIG. 27 is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:14 or the sequence depicted in FIGS. 28A-28B is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:15 or the sequence depicted in FIGS. 29A-29B is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:16 or the sequence depicted in FIGS. 30A-30B is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:17 or the sequence depicted in FIGS. 31A-31B is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:18 or the sequence depicted in FIGS. 32A-B is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:19 or the sequence depicted in FIGS. 33A-B is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:20 or the sequence depicted in FIGS. 34A-B is administered to a subject having cancer. In some embodiments, a therapeutic amount of an engineered immune cell or a population thereof (e.g., engineered CD3+ T cell, engineered CD4+ T cell, or engineered CD8+ T cell) comprising a nucleic acid construct comprising SEQ ID NO:21 or the sequence depicted in FIGS. 35A-B is administered to a subject having cancer. In some embodiments, a therapeutic amount of an engineered immune cell (e.g., engineered CD3+ T cell, engineered CD4+ T cell, or engineered CD8+ T cell) or a population thereof comprising a nucleic acid construct comprising SEQ ID NO:22 or the sequence depicted in FIGS. 36A-B is administered to a subject having cancer. In some embodiments, a therapeutic amount of an engineered immune cell (e.g., engineered CD3+ T cell, engineered CD4+ T cell, or engineered CD8+ T cell) or a population thereof comprising a nucleic acid construct comprising SEQ ID NO:23 or the sequence depicted in FIGS. 37A-B is administered to a subject having cancer.

In some embodiments, a therapeutic amount of a population of engineered immune cells (e.g., engineered CD3+ T cells, engineered CD4+ T cells, or engineered CD8+ T cells) comprising a CD7 PEBL of SEQ ID NO:25 is administered to a subject with cancer. In some embodiments, a therapeutic amount of a population of engineered immune cells (e.g., engineered CD3+ T cells, engineered CD4+ T cells, or engineered CD8+ T cells) comprising a CD7 PEBL of SEQ ID NO:27 is administered to a subject with cancer.

In some embodiments, a therapeutic amount of a population of engineered immune cells (e.g., engineered CD3+ T cells, engineered CD4+ T cells, or engineered CD8+ T cells) comprising a CD7 CAR of SEQ ID NO:29 is administered to a subject with cancer. In some embodiments, a therapeutic amount of a population of engineered immune cells (e.g., engineered CD3+ T cells, engineered CD4+ T cells, or engineered CD8+ T cells) comprising a CD7 CAR of SEQ ID NO:31 is administered to a subject with cancer.

In some embodiments, a therapeutic amount of a population of engineered immune cells (e.g., engineered CD3+ T cells, engineered CD4+ T cells, or engineered CD8+ T cells) comprising a CD7 PEBL of SEQ ID NO:25 and a CD7 CAR of SEQ ID NO:29 is administered to a subject with cancer. In some embodiments, a therapeutic amount of a population of engineered immune cells (e.g., engineered CD3+ T cells, engineered CD4+ T cells, or engineered CD8+ T cells) comprising a CD7 PEBL of SEQ ID NO:27 and a CD7 CAR of SEQ ID NO:29 is administered to a subject with cancer.

In some embodiments, a therapeutic amount of a population of engineered immune cells (e.g., engineered CD3+ T cells, engineered CD4+ T cells, or engineered CD8+ T cells) comprising a CD7 PEBL of SEQ ID NO:25 and a CD7 CAR of SEQ ID NO:31 is administered to a subject with cancer. In some embodiments, a therapeutic amount of a population of engineered immune cells (e.g., engineered CD3+ T cells, engineered CD4+ T cells, or engineered CD8+ T cells) comprising a CD7 PEBL of SEQ ID NO:27 and a CD7 CAR of SEQ ID NO:31 is administered to a subject with cancer.

In some embodiments, a therapeutic amount of a population of engineered immune cells (e.g., engineered CD3+ T cells, engineered CD4+ T cells, or engineered CD8+ T cells) is administered to a subject with cancer, wherein the engineered immune cells comprise SEQ ID NO:95.

In certain embodiments, the cancer is a T cell malignancy, e.g., T cell leukemia or T cell lymphoma, such a T-cell acute lymphoblastic leukemia, T-cell prolymphocytic leukemia, T-cell large granular lymphocytic leukemia, enteropathy-associated T-cell lymphoma, hepatosplenic T-cell lymphoma, subcutaneous panniculitis-like T-cell lymphoma, mycosis fungoides, Sezary syndrome, primary cutaneous gamma-delta T-cell lymphoma, peripheral T-cell lymphoma not otherwise specified, angioimmunoblastic T-cell lymphoma, anaplastic large cell lymphoma. In certain embodiments, the T cell malignancy is early T-cell progenitor acute lymphoblastic leukemia (ETP-ALL).

In some embodiments, the engineered immune cell is autologous to the subject in need of treatment, e.g., cancer treatment. In other embodiments, the engineered immune cell is allogenic to the subject in need of treatment.

In certain embodiments, the engineered immune cell is administered into the subject by intravenous infusion, intra-arterial infusion, direct injection into tumor and/or perfusion of tumor bed after surgery, implantation at a tumor site in an artificial scaffold, intrathecal administration, and intraocular administration.

In certain embodiments, the engineered immune cell is administered by infusion into the subject. Methods of infusing immune cells (e.g., allogeneic or autologous immune cells) are known in the art. A sufficient number of cells are administered to the recipient in order to ameliorate the symptoms of the disease. Typically, dosages of 10⁷ to 10¹⁰ cells are infused in a single setting, e.g., dosages of 10⁹ cells. Infusions are administered either as a single 10⁹ cell dose or divided into several 10⁹ cell dosages. The frequency of infusions can be daily, every 2 to 30 days or even longer intervals if desired or indicated. The quantity of infusions is generally at least 1 infusion per subject and preferably at least 3 infusions, as tolerated, or until the disease symptoms have been ameliorated. The cells can be infused intravenously at a rate of 50-250 ml/hr. Other suitable modes of administration include intra-arterial infusion, intraperitoneal infusion, direct injection into tumor and/or perfusion of tumor bed after surgery, implantation at the tumor site in an artificial scaffold, intrathecal administration. Methods of adapting the present invention to such modes of delivery are readily available to one skilled in the art.

In certain embodiments, the method of treating cancer according to the present invention is combined with at least one other known cancer therapy, e.g., radiotherapy, chemotherapy, or other immunotherapy.

In other aspects, also provided is use of an engineered immune cell having any of the embodiments described herein for treating cancer, comprising administering a therapeutic amount of the engineered immune cell to a subject in need thereof. In certain embodiments, the cancer is a T cell malignancy. In certain embodiments, the T cell malignancy is early T-cell progenitor acute lymphoblastic leukemia (ETP-ALL).

In certain embodiments, the engineered immune cell is administered into the subject by intravenous infusion, intra-arterial infusion, intraperitoneal infusion, direct injection into tumor and/or perfusion of tumor bed after surgery, implantation at a tumor site in an artificial scaffold, and intrathecal administration.

### EXAMPLES

### Example 1: Using Bicistronic Expression Vectors for Blockade of CD7 Expression in Chimeric Antigen-Receptor T-Cells

This example illustrates blockade of CD7 expression in anti-CD7 CAR-T cells using bicistronic expression constructs.

### Methods

### Cell Culture

293T cells (ATCC CRL-3216) were maintained in DMEM (Gibco) with 10% FBS (Hyclone), 100 U/mL penicillin and 100 ug/mL streptomycin (Gibco). Jurkat clone E6-1 cells (ATCC TIB-152) and NALM6 clone G5 cells (CRL-3273) were maintained in RPMI1640 (Gibco) with 10% FBS (Hyclone), 100 U/mL penicillin, 100 ug/mL streptomycin (Gibco) and 1× GlutaMAX (Gibco).

### Lentivirus Production

293T cells were cotransfected with lentiviral transfer vectors and Virapower packaging plasmids mix (Invitrogen) at a ratio of 1:3 using Lipofectamine 2000 (Invitrogen). Transfection medium was replaced with fresh DMEM (Gibco) with 10% FBS (Hyclone) 6 hours post transfection. 48 h later, the virus supernatant was collected, passed through a 0.45p M filter and then concentrated 100× using Lenti-X Concentrator (Clontech). Concentrated lentivirus stock was stored at −150° C. until use.

### Retrovirus Production

293T cells were cotransfected with retroviral transfer vectors and pEQ and pRDF packaging plasmids using X-tremeGENE 9 DNA transfection reagent (Roche). Transfection medium was replaced with fresh DMEM (Gibco) with 10% FBS (Hyclone) 6 hours post transfection. 24 h and 48 h later, the virus supernatant was collected and passed through a 0.45p M filter. Retrovirus was used fresh or stored at −150° C. until use.

### Lentivirus Titration on 293T Cells

293T cells were transduced with varying volumes of lentiviruses in the presence of 5 μg/mL polybrene (Sigma). After 15 h overnight culture, transduction medium was removed and cells were treated with 10 U/mL DNaseI (New England Biolabs) in fresh culture media for 15 min at 37° C. The media was then replaced with fresh DMEM with 10% FBS for further culture. Transduced cells were harvested for analysis at ≥72 h post transduction. Virus titers were determined using flow cytometry and RT-qPCR.

Transducing unit (TU) titers were calculated from flow cytometry data using the equation: [TU/mL=(Number of 293T cells per sample×% CAR+ cells)÷Virus volume in mL]. TU titers were calculated using samples within the linear range of % CAR+ cells and virus volume.

Integration unit (IU) titers were calculated from RT-qPCR data on genomic DNA using the equation: [IU/ml=(Number of 293T cells per sample×number of proviral gene copies per genome)÷Virus volume in mL]. IU titers were calculated using samples within the linear range of proviral gene copy number and virus volume.

### Lentiviral Transduction of Jurkat Cells

Lentiviruses were directly added to Jurkat cells with or without 8 μg/mL polybrene (Sigma). A complete media change was performed two days later to remove lentiviruses from cultures. Transduced cells were harvested for analysis at ≥2 days post transduction.

### Primary T Cell Culture

Frozen human primary peripheral blood mononuclear cells (PBMCs) (ATCC Cat #PCS-800-011 and Stemcell Technologies Cat #70025) were thawed, recovered overnight, and maintained at 1 million cells/mL in either RPMI1640 (Gibco) with 10% FBS (Hyclone), 100 U/mL penicillin, 100 ug/mL streptomycin (Gibco) and 1× GlutaMAX (Gibco), TexMACS medium (Miltenyi Biotec) supplemented with 3% human AB serum (Sigma), or serum-free TexMACS medium. Culture media was supplemented with 120 IU/mL Interleukin-2 (Miltenyi Biotec) every 2 to 3 days.

PBMCs were either cultured in bulk without further selection or purified for T cells after overnight recovery. CD4+ and CD8+ T cells were isolated using CD4 Microbeads (Miltenyi Biotec) and CD8 Microbeads (Miltenyi Biotec). CD3+ T cells were isolated using CD3 Microbeads (Miltenyi Biotec).

T cells were activated with either T Cell TransAct (Miltenyi Biotec) or Dynabeads Human T-Activator CD3/CD28 for T Cell Expansion and Activation (Gibco) according to manufacturers' recommendations. Dynabeads were added at a bead to cell ratio of 1:1. Bead depletion was performed after 4 days.

Lentiviral Transduction of Primary T Cells

Primary T cells were transduced at 1 to 4 days post activation. Static transduction was performed where lentiviruses were directly added to T cells. A complete media change was performed two days later to remove lentiviruses from cultures. Transduced cells were analysed by flow cytometry at ≥3 days post transduction.

### Retroviral Transduction of Primary T Cells

Retronectin-based retroviral transduction was performed on primary T cells. Non-treated tissue culture plates were coated with 2.5 μg/cm2 of RetroNectin Recombinant Human Fibronectin Fragment (Takara) according to manufacturer's recommendations. Retrovirus supernatants were added to retronectin-coated plates and centrifuged at 1000×g for 2 h at 32° C. Virus supernatants were then removed from the wells. Wells were rinsed with culture media before adding T cells. Transduced cells were analysed by flow cytometry at ≥3 days post transduction.

### Flow Cytometry

Antibody staining and washes are performed with staining buffer (1×PBS pH 7.4, 0.2% BSA, 0.02% sodium azide). Cells were incubated with antibodies on ice for 15 min and washed 3 times. The following antibodies were used for staining: anti-mouse F(ab′)₂-biotin (Jackson Immunoresearch 115-066-072), streptavidin-APC (Jackson Immunoresearch 016-130-084), anti-human CD7-PE (BD 555361), anti-human CD3 eFluor780 (eBioscience 47-0037-42). Cells stained with anti-mouse F(ab′)₂-biotin were blocked with 3 g of mouse IgG1 isotype control antibody (BioXCell) for 5 min before adding the remaining antibodies. DAPI was used at 1 g/mL for live/dead discrimination. Stained cells were collected on an Invitrogen Attune NxT flow cytometer and analyzed with FlowJo v10 software.

### Real-Time Quantitative PCR (RT-qPCR)

Genomic DNA was extracted from cells using the DNeasy Blood and Tissue Kit (Qiagen) and RNase A (Qiagen). Total RNA was extracted from cells using the MN NucleoSpin RNA Kit (Macherey-Nagel), and cDNA was synthesized using the Maxima First Strand cDNA Synthesis Kit (Thermo Scientific). All kits were used according to manufacturers' recommendations. RT-qPCR was performed using iTaq Universal SYBR Green Supermix (Bio-Rad) on a CFX96 Touch™ Real-Time PCR Detection System (Bio-Rad).

Primers Used were:

| | |
|---|---|
| RPPH1-F: | |
| 5′-GAGGGAAGCTCATCAGTGGG-3′ | (SEQ ID NO: 87) |
| RPPH1-R: | |
| 5′-CATCTCCTGCCCAGTCTGAC-3′ | (SEQ ID NO: 88) |
| WPRE-F: | |
| 5′-CCTTTCCGGGACTTTCGCTTT-3′ | (SEQ ID NO: 89) |
| WPRE-R: | |
| 5′-GCAGAATCCAGGTGGCAACA-3′ | (SEQ ID NO: 90) |
| TH69CD7CAR-F: | |
| 5′-GCAGCCTTTCATGAGACCAG-3′ | (SEQ ID NO: 91) |
| TH69CD7CAR-R: | |
| 5′-TGCCCAGGTTCAGCTCATTA-3′ | (SEQ ID NO: 92) |
| TH69CD7PEBL-F: | |
| 5′-ACCTGCCGCATACAAGGATA-3′ | (SEQ ID NO: 93) |
| TH69CD7PEBL-R: | |
| 5′-CCACTGTGCAGACTAGAGGT-3′ | (SEQ ID NO: 94) |

All assay primers had primer efficiencies between 90% and 110%.

Fold changes of all genes were normalized to a housekeeping gene using the equation: [Fold change=2\hat{ }−(Ct (target gene)−Ct (housekeeping gene))]. Copy number of target genes was normalized to the genomic copy number of RNaseP in 293T cells.

### Western Blot

Cells were lysed with RIPA buffer (Pierce) and protease inhibitor (Pierce). Protein quantitation of cell lysates was performed using a Bradford Coomassie protein assay kit (Thermo Scientific), according to manufacturer's recommendations. Western blots were performed using an automated western blot system, Simple Western Wes (ProteinSimple), with a 12-230 kDa Wes Separation Module. The following primary antibodies were used: anti-β-Actin, clone 13E5 (Cell Signaling Technology); anti-Myc-Tag, clone 71D10 (Cell Signaling Technology); and anti-CD3ζ polyclonal antibody, Cat #ab226475 (Abcam). Secondary antibodies from Wes Anti-Rabbit Detection Module were used. The data was analysed with Compass for Simple Western software.

### IFNγ Secretion

Effector CAR-T cells were resuspended at a cell density of 10⁶ cells/mL, and 100,000 CAR-T cells were plated per well in a 96-well round-bottom plate. Target cells were cocultured with effector CAR-T cells at various effector:target (E:T) ratios for 24 h. After 24 h, the cells were spun down and supernatants were collected and stored at −150° C. The supernatants were evaluated for IFNγ secretion using the ELISA MAX Standard Set Human IFN-γ kit (Biolegend) according to manufacturer's recommendations.

### Cytotoxicity Assay

Target cells were resuspended at a cell density of 10⁶ cells/mL and loaded with 0.4 μg/ml of calcein red-orange AM (Invitrogen) for 10 min. Loaded cells were then washed thrice to remove excess calcein. 100,000 target cells were plated per well in a 96-well round-bottom plate. Target cells were cocultured with effector CAR-T cells at various effector:target (E:T) ratios for 4 h. After 4 h, DAPI was added to all wells, and cells were collected on the flow cytometer. The number of remaining live target cells was counted in all wells. The percentage cytotoxicity was calculated with the following equation:

Percentage cytotoxicity=[(S−E)/S]*100%

S=Remaining live target cells in target cell only control wells

E=Remaining live target cells after coculture with effector T cells in experimental wells

### Results

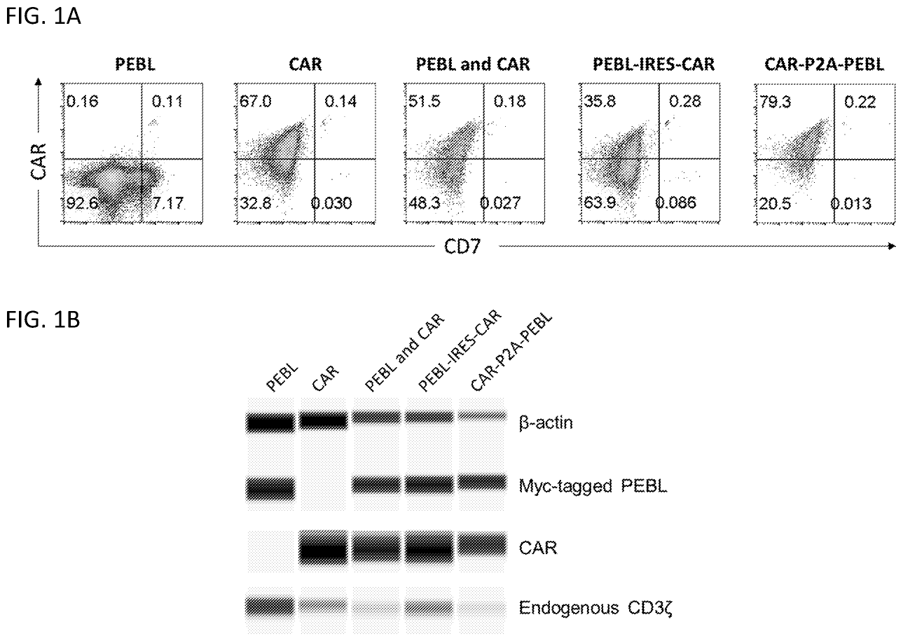

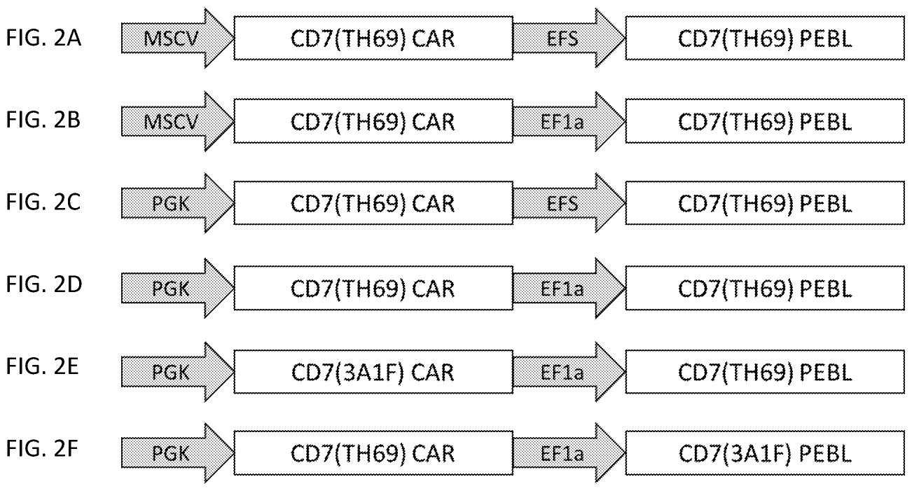

Primary T cells were transduced with the different retroviruses expressing (1) PEBL; (2) CAR; (3) PEBL and CAR sequentially; (4) PEBL-IRES-CAR; or (5) CAR-P2A-PEBL. The transduced cells were analyzed by flow cytometry for CD7 and CAR expression (FIG. 1A). Cell lysates from primary T cells transduced with the indicated retroviruses were analyzed by Western blot for β-actin, Myc-tagged PEBL, CAR and endogenous CD3ζ expression (FIG. 1).

Dual promoter lentiviral constructs were prepared to express an anti-CD7 CAR and an anti-CD7 PEBL from a single vector. As shown in FIG. 2A-FIG. 2F, the general format of the dual promoter construct from 5′ end to 3′ end included a first promoter—an anti-CD7 CAR—a second promoter—an anti-CD7 PEBL. The promoters tested include a MSCV promoter, an EFS promoter, a PGK promoter, and an EF1a promoter. Nucleic acid sequences of exemplary dual promoter constructs are provided as SEQ ID NOS:19-23 and shown in FIGS. 33A-33B, FIGS. 34A-34B, FIGS. 35A-35B, FIGS. 36A-36B, and FIGS. 37A-37B. Such constructs encoded anti-CD7 CARs including an anti-CD7 CAR based on the TH69 antibody and an anti-CD7 CAR based on the 3A1F antibody, as well as anti-CD7 PEBLs including an anti-CD7 PEBL based on the TH69 antibody and an anti-CD7 PEBL based on the 3A1F antibody.

The dual promoter lentiviral vectors were transduced into cells to produce cells with partial downregulation of surface CD7 expression and low expression of the anti-CD7 CAR. The percentage of cells expressing the CAR-remained low after an extended time in culture. FIG. 3 shows the expression of the CAR (y-axis) and the expression of CD7 (x-axis) in the cells at 5 days after transduction and at 14 days after transduction. The figure shows expression of cells (e.g., healthy donor cells including healthy donor lymphocytes) transduced with the exemplary dual promoter constructs are provided as SEQ ID NOS:18-23 and shown in FIGS. 32A-32B, 33A-33B, FIGS. 34A-34B, FIGS. 35A-35B, FIGS. 36A-36B, and FIGS. 37A-37B. As an example, the cells transduced with the dual promoter lentiviral vector comprising the MSCV promoter-CD7 (TH69) CAR-EF1a promoter-CD7 (TH69) PEBL produced a population of cells comprising CD7 CAR-neg/CD7-neg cells (52.8%), CD7 CAR+/CD7-neg cells (2.98%), CD7 CAR-neg/CD7+ cells (40.4%), and CD7 CAR+/CD7+ cells (3.84%) at 5 days post transduction. At 14 days post transduction the MSCV promoter-CD7 (TH69) CAR-EF1a promoter-CD7 (TH69) PEBL transduced cells included a population of cells comprising CD7 CAR-neg/CD7-neg cells (42.6%), CD7 CAR+/CD7-neg cells (0.14%), CD7 CAR-neg/CD7+ cells (54.9%), and CD7 CAR+/CD7+ cells (2.37%).

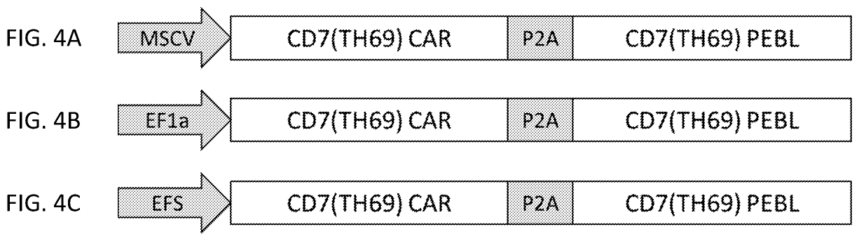

In an effort to optimize expression of the CD7 CAR and CD7 PEBL single promoter bicistronic lentiviral constructs were generated. Exemplary schematic diagrams of such constructs are provided in FIG. 4A, FIG. 4B, and FIG. 4C. FIG. 4A depicts a schematic of an exemplary bicistronic construct comprising an MSCV promoter-anti-human CD7 (TH69) CAR-P2A-anti-human CD7 (TH69) PEBL, such as the one of SEQ ID NO:14. FIG. 4B depicts a schematic of an exemplary bicistronic construct comprising an EF1a promoter-anti-human CD7 (TH69) CAR-P2A-anti-human CD7 (TH69) PEBL, such as the one of SEQ ID NO:15. FIG. 4c depicts a schematic of an exemplary bicistronic construct comprising an EFS promoter-anti-human CD7 (TH69) CAR-P2A-anti-human CD7 (TH69) PEBL, such as the one of SEQ ID NO:16.

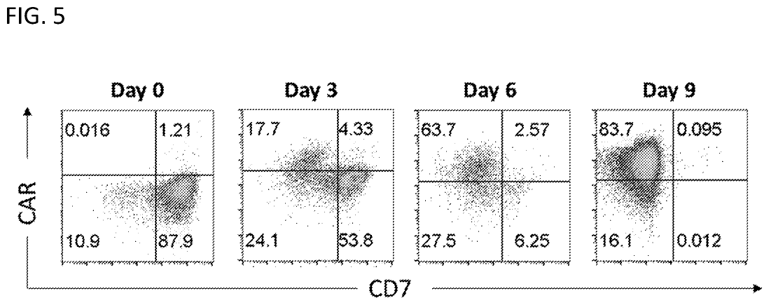

Cells transduced with an MSCV promoter-anti-human CD7 (TH69) CAR-P2A-anti-human CD7 (TH69) PEBL lentivirus generated a population of CD7 CAR+/CD-neg T cells. FIG. 5 shows an expansion and enrichment of the CD7 CAR+/CD7-neg T cells from day 0 to day 9 post transduction. For example at day 0, 10.9% of the cells were CD7 CAR-neg/CD7-neg cells, 0.016% were CD7 CAR+/CD7-neg cells, 87.9% were CD7 CAR-neg/CD7+ cells, and 1.21% were CD7 CAR+/CD7+ cells. At day 3, 24.1% of the cells were CD7 CAR-neg/CD7-neg cells, 17.7% were CD7 CAR+/CD7-neg cells, 53.8% were CD7 CAR-neg/CD7+ cells, and 4.33% were CD7 CAR+/CD7+ cells. At day 6, 27.5% of the cells were CD7 CAR-neg/CD7− cells, 63.7% were CD7 CAR+/CD7-neg cells, 6.25% were CD7 CAR-neg/CD7+ cells, and 2.57% were CD7 CAR+/CD7+ cells. At day 9, 16.1% of the cells were CD7 CAR-neg/CD7− neg cells, 83.7% were CD7 CAR+/CD7− cells, 0.012% were CD7 CAR-neg/CD7+ cells, and 0.095% were CD7 CAR+/CD7+ cells.

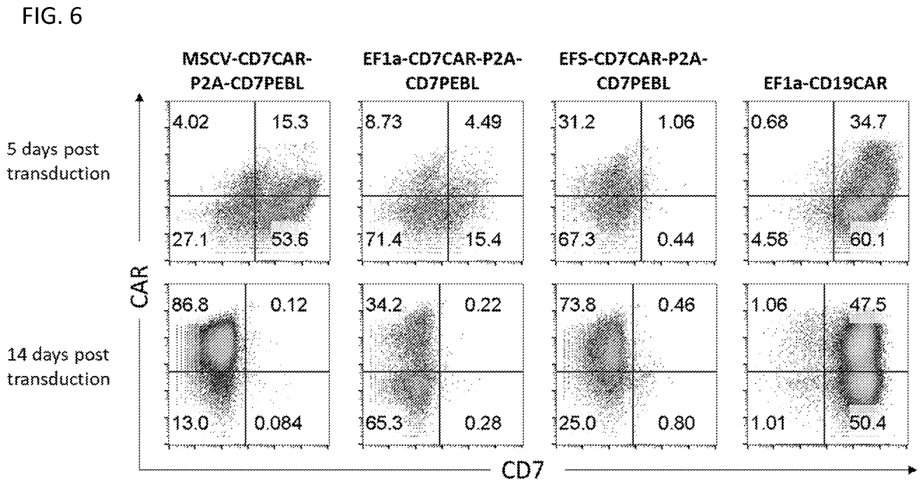

Cells transduced with an anti-human CD7 (TH69) CAR-P2A-anti-human CD7 (TH69) PEBL bicistronic lentivirus vectors with different promoters produced CD7 CAR+/CD7-neg cells that were enriched in culture over time. FIG. 6 shows an increase in the percentage of CD7 CAR+/CD7-neg cells at 5 days post transduction and 14 days post transduction. For example, cell transduced with an EFS promoter anti-human CD7 (TH69) CAR-P2A-anti-human CD7 (TH69) PEBL lentiviral vector produced 67.3% CD7 CAR-neg/CD7− cells, 31.2% were CD7 CAR+/CD7-neg cells, 0.44% were CD7 CAR-neg/CD7+ cells, and 1.06% were CD7 CAR+/CD7+ cells at 5 days post transduction. By 14 days post transduction 25.0% CD7 CAR-neg/CD7− cells, 73.8% were CD7 CAR+/CD7-neg cells, 0.80% were CD7 CAR-neg/CD7+ cells, and 0.46% were CD7 CAR+/CD7+ cells. As a control, cells were transduced with a lentiviral vector containing a EF1a promoter upstream of an anti-CD19 CAR at 5 days post transduction 34.7% of the cells were CD19 CAR+/CD7+, 60.1% were CD19 CAR-neg/CD7+, 4.58% were double negative, and 0.68% were CD19 CAR+/CD7-neg. And at 14 days post transduction, 47.5% of the cells were CD19 CAR+/CD7+, 50.4% were CD19 CAR-neg/CD7+, 1.01% were double negative, and 1.06% were CD19 CAR+/CD7-neg. Thus, almost no enrichment of CD19 CAR expressing cells was detected over time.

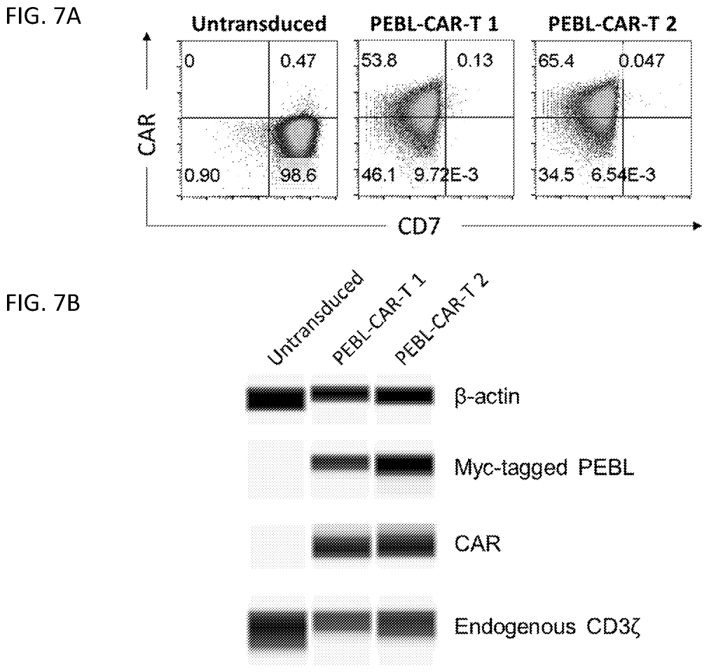

To evaluate consistency and reproducibility of the single promoter bicistronic vectors, cells were transduced with two independent lots of an MSCV promoter-anti-human CD7 (TH69) CAR-P2A-anti-human CD7 (TH69) PEBL lentivirus. The CD7 PEBL was myc tagged and detected by Western blot. The CD7 CAR was also detected (FIG. 7B). FIG. 7A shows flow cytometry analysis of the CD7 CAR and CD7 expression in the transduced cells. The first lot generated a population of transduced cells comprising 53.8% CD7 CAR+/CD7-neg cells and 46.1% CD7-neg/CD7-neg cells. The second lot produced a population of transduced cells comprising 65.5% CD7 CAR+/CD7-neg cells and 34.5% CD7-neg/CD7-neg cells. It was noted that the untransduced cells included 98.6% CAR-neg/CD7+ cells.

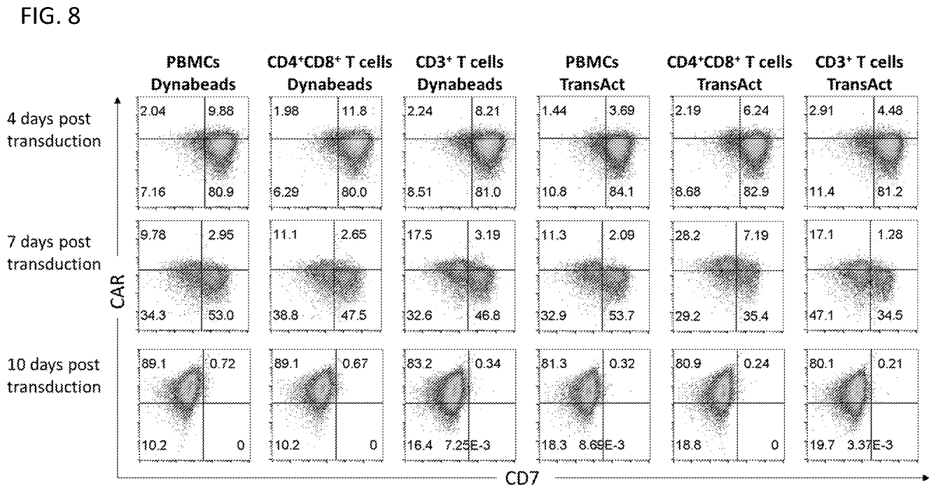

It should be noted that the single promoter bicistronic vectors described herein were successfully used to produce CD7 PEBL-CAR-T cells from different starting cells including bulk PBMCs, purified T cells comprising CD4+ and CD8+ T cells, and purified CD3+ T cells. In addition, different T cell activation reagents including Dynabeads® Human T-Activator CD3/CD28 for T Cell Expansion and Activation (Gibco) and T Cell TransAct™ (Miltenyi Biotec) were used. FIG. 8 shows an increasing percentage of CD7 CAR+/CD7-neg T cells when the anti-human CD7 (TH69) CAR-P2A-anti-human CD7 (TH69) PEBL transduced cells were cultured over time. A comparable expansion of the CD7 CAR+/CD7-neg T cells was detected among the different starting cell types and between the two activation reagents.

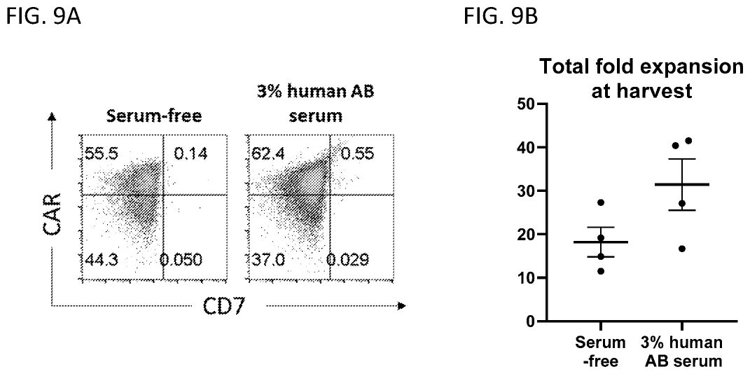

The cells described herein (e.g., CD7 CAR+/CD7-neg T cells) were generated from purified CD4+ positively selected and CD8+ positively selected T cells cultured in either serum-free TexMACS media or TexMACS media supplemented with 3% human AB serum. T cells were transduced with CD7CAR-P2A-CD7PEBL lentivirus at MOI 10 to generate CD7-CAR+ T cells. Total fold change of transduced cells at 11 days post cell activation was higher with serum-supplemented media (FIG. 9A and FIG. 9B).

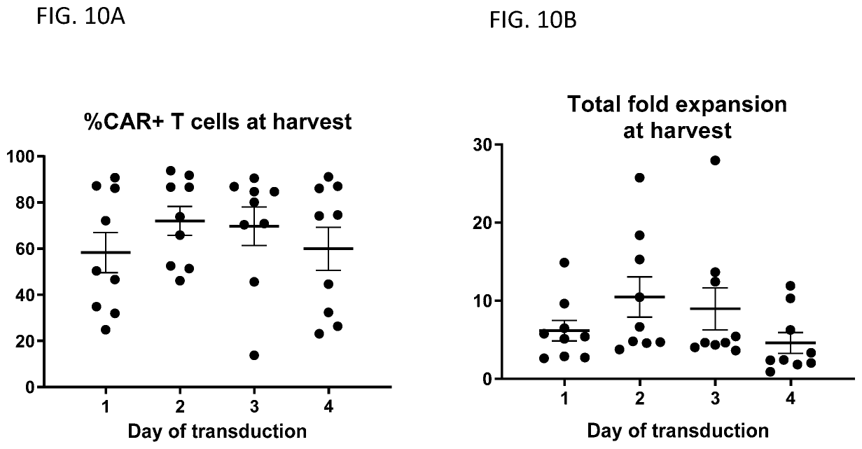

Purified CD4+ and CD8+ selected T cells transduced with CD7CAR-P2A-CD7PEBL lentivirus at different days post activation (day 1 to day 4) generated a highly pure population of CAR+ T cells (FIG. 10A and FIG. 10B). Cells transduced on different days expanded and proliferated during the manufacturing process. FIG. 10B shows that the transduced T cells exhibited about an average of 5-fold to 10-fold expansion when the cells were transduced at 1, 2, 3, or 4 days after activation.

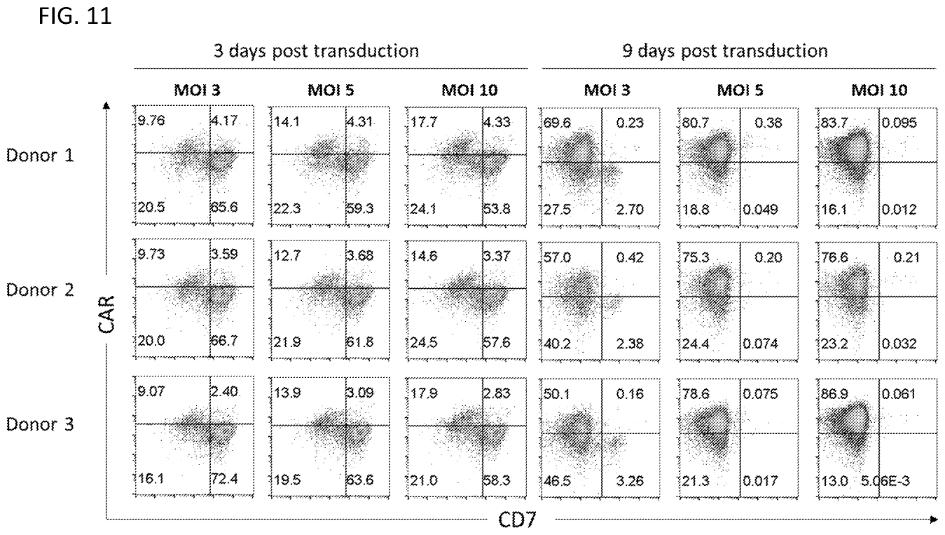

Expression of the CAR and endogenous CD7 in T cells transduced with different MOIs of the CD7CAR-P2A-CD7PEBL lentivirus was measured by FACS. T cells transduced at lower MOIs had a lower percentage of CD7 CAR+/CD7-neg T cells early in the transduction process, but increased to match the percentage of CD7 CAR+/CD7-neg T cells obtained at higher MOIs of transduction (see, e.g., FIG. 11). For instance, T cells from Donor 1 that were transduced with the lentivirus at an MOI of 3 were 9.76% CD7 CAR+/CD7-neg T cells at 3 days post transduction and 69.9% CD7 CAR+/CD7-neg T cells at 9 days post transduction. T cells from Donor 1 that were transduced with the lentivirus at an MOI of 10 were 17.7% CD7 CAR+/CD7-neg at 3 days post transduction and 83.7% CD7 CAR+/CD7-neg at 9 days post transduction.

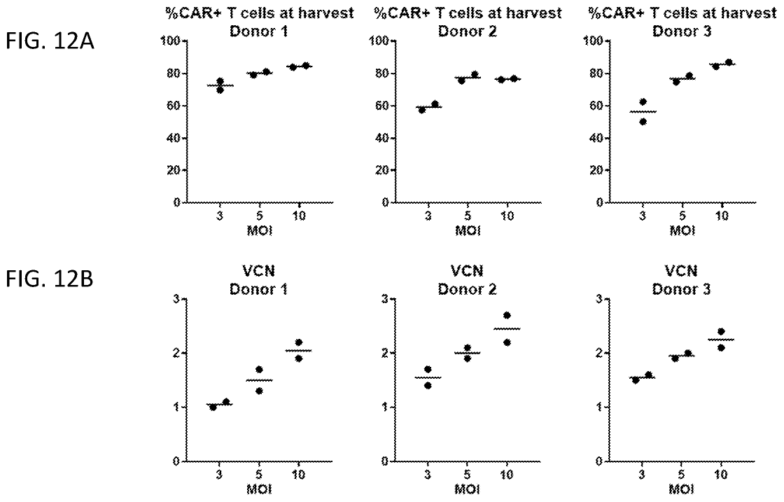

Purified CD4+ and CD8+ T cells from three unique donors were transduced with CD7CAR-P2A-CD7PEBL lentivirus at the indicated MOI in two individual wells. The percentage of CAR+ cells were analysed by flow cytometry. Cell pellets were collected and genomic DNA was extracted to determine vector copy number (VCN) by RT-qPCR analysis. Higher MOI correlated with higher VCN (FIG. 12B), however the percentage of CD7 CAR+ T cells was similar at MOI 5 and 10 (FIG. 12A).

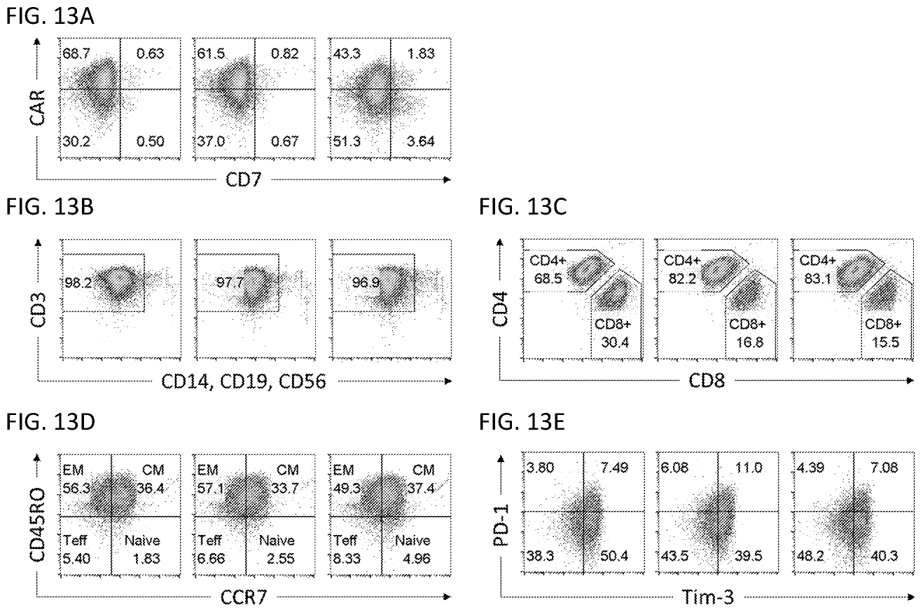

Expression of various surface markers was measured in the primary T cells transduced with MSCV-CD7CAR-P2A-CD7PEBL lentivirus at 11 days post activation. FIG. 13A shows CD7 CAR and endogenous CD7 expression in transduced cells from three different donors. Expression of CD3 compared to CD14/CD19/CD56 is shown in FIG. 13B. CD4 and CD8 expression is shown in FIG. 13C. FIG. 13D shows that transduced cells generated different subsets of T cells including T_{EM} cells, T_{CM} cells, Teff cells, and naïve T cells as determined by CD45RO and CCR7 expression. FIG. 13D shows PD-1 and TIM-3 expression in the transduced T cells.

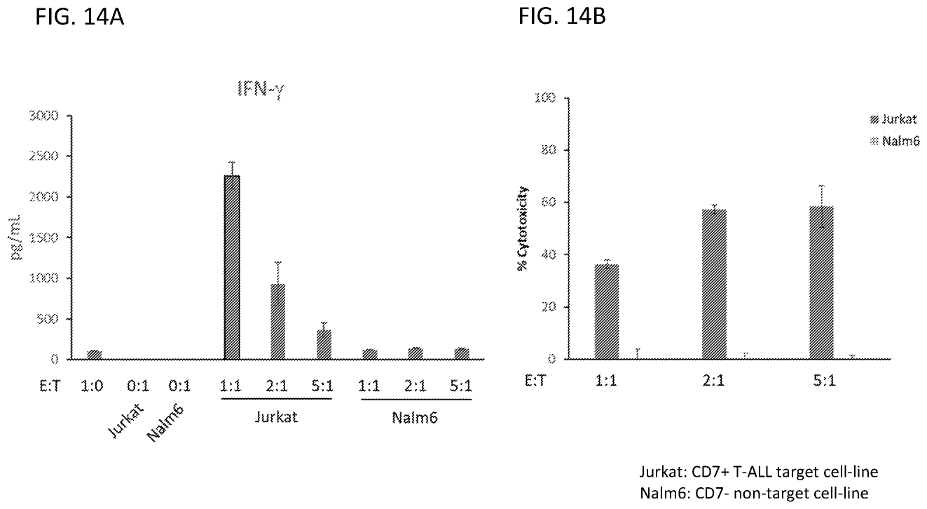

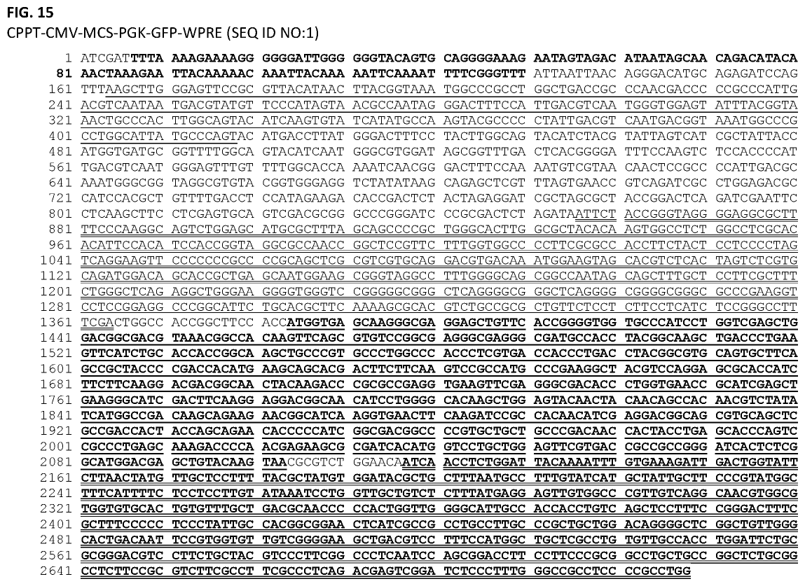

The response of the transduced PEBL-CAR T cells to CD7+ Jurkat cells and CD7-negative Nalm6 cells was determined by IFNγ secretion (FIG. 14A) and cytotoxicity (FIG. 14B). IFN-g secretion measured in culture supernatant of PEBL-CAR T cells co-cultured with Jurkat or Nalm6 cells at the indicated E:T ratios for 24 h (mean±SD of technical replicates). PEBL-CAR-T cells showed target-specific functional responses, as IFNγ was secreted by the PEBL-CAR-T cells when cultured with CD7+ Jurkat cells, and not with Nalm6 cells. In addition, PEBL-CAR-T cells killed CD7+ Jurkat cells but not CD7-negative Nalm6 cells in a cytotoxicity assay.

This example demonstrates the generation and expansion of PEBL-CAR-T cells produced using a CD7 CAR-P2A-CD7 PEBL biscistronic lentiviral vector. Such cells showed antigen-specific T cell functional responses such as IFNγ secretion and specific toxicity against CD7+ target cell lines. The PEBL-CAR-T cells exhibited high percentage purity of CD7 negative, CAR+ T cells.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A bicistronic retroviral vector comprising:
(a) a first polynucleotide encoding an anti-CD7 chimeric antigen receptor (CAR) comprising at least 90% sequence identity to the amino acid sequence of any one of SEQ ID NOS:28-31;
(b) a second polynucleotide encoding an Internal Ribosome Entry Site (IRES) or a ribosomal codon skipping site; and
(c) a third polynucleotide encoding an anti-CD7 protein expression blocker (PEBL) comprising at least 90% sequence identity to the amino acid sequence of SEQ ID NOS:24-27;
wherein the first polynucleotide is operably linked the second polynucleotide which is operably linked the third polynucleotide.

2. The bicistronic retroviral vector of claim 1, wherein the anti-CD7 CAR comprises the amino acid sequence of any one of SEQ ID NOS:28-31.

3. The bicistronic retroviral vector composition of claim 1 or 2, wherein the anti-CD7 PEBL comprises the amino acid sequence of any one of SEQ ID NOS:24-27.

4. The bicistronic retroviral vector composition of any one of claims 1 to 3, wherein the anti-CD7 CAR comprises the amino acid sequence of SEQ ID NO:29 and the anti-CD7 PEBL comprises the amino acid sequence of SEQ ID NO:25.

5. The bicistronic retroviral vector composition of any one of claims 1 to 3, wherein the anti-CD7 CAR comprises the amino acid sequence of SEQ ID NO:31 and the anti-CD7 PEBL comprises the amino acid sequence of SEQ ID NO:27.

6. The bicistronic retroviral vector of any one of claims 1 to 5, wherein the IRES is derived from Encephalomyocarditis virus (EMCV) or an Enterovirus.

7. The bicistronic retroviral vector of any one of claims 1 to 5, wherein the ribosomal codon skipping site comprises a 2A self-cleaving peptide.

8. The bicistronic retroviral vector of claim 7, wherein the 2A self-cleaving peptide is selected from the group consisting of a F2A peptide (foot-and-mouth disease virus 2A peptide), an E2A peptide (equine rhinitis A virus 2A peptide), a P2A peptide (porcine teschovirus-1 2A peptide), and a T2A peptide (thosea asigna virus 2A).

9. The bicistronic retroviral vector of any one of claims 1 to 6, comprising at least 90% sequence identity to the nucleic acid sequence of SEQ ID NO:12.

10. The bicistronic retroviral vector of any one of claims 1 to 5 and 6, comprising at least 90% sequence identity to the nucleic acid sequence of SEQ ID NO:13.

11. The bicistronic retroviral vector of claim 10, comprising the nucleic acid sequence of SEQ ID NO:13.

12. The bicistronic retroviral vector of any one of claims 1 to 11, further comprises a promoter element.

13. The bicistronic retroviral vector of claim 12, wherein the promoter element is selected from the group consisting of a CMV promoter, EF1α promoter, EFS promoter, MSCV promoter, and PGK promoter.

14. The bicistronic retroviral vector of claim 13, wherein the promoter element comprises at least 90% sequence identity to the nucleic acid sequence of any one selected from the group consisting of SEQ ID NOS:6-10.

15. The bicistronic retroviral vector of claim 13 or 14, wherein the promoter element comprises the nucleic acid sequence of any one selected from the group consisting of SEQ ID NOS:6-10.

16. The bicistronic retroviral vector of any one of claims 12 to 15, comprising at least 90% sequence identity to the nucleic acid sequence of any one selected from the group consisting of SEQ ID NOS:14-16.

17. The bicistronic retroviral vector of any one of claims 12 to 16, comprising the nucleic acid sequence of any one selected from the group consisting of SEQ ID NOS:14-16.

18. The bicistronic retroviral vector of any one of claims 1 to 18, wherein the retroviral vector is a lentiviral vector.

19. An engineered immune cell comprising the bicistronic retroviral vector of any one of claims 1 to 18.

20. The engineered immune cell of claim 19, wherein the engineered immune cell is an allogeneic T cell.

21. The engineered immune cell of claim 20, wherein the engineered immune cell is an autologous T cell.

22. The engineered immune cell of claim 20 or 21, wherein the engineered immune cell has reduced CD7 surface expression compared to a corresponding immune cell and expresses the anti-CD7 CAR.

23. A pharmaceutical composition comprising the engineered immune cell of claim 19 and a pharmaceutically effective carrier.

24. A method of treating a cancer in a subject comprising administering a therapeutically effective amount of the engineered immune cell of any one of claims 19 to 21, or the pharmaceutical composition of claim 23.

25. A method of producing an engineered immune cell comprising transducing an immune cell with the bicistronic retroviral vector of any one of claims 1 to 18, and recovering the engineered immune cell.

26. The method of claim 25, wherein the immune cell is selected from the group consisting of a peripheral blood mononuclear cell, an isolated CD4+ T cell, an isolated CD8+ T cell, and an isolated CD3+ T cell.

27. The method of claim 25 or 26, wherein the engineered immune cell has reduced CD7 surface expression compared to a corresponding immune cell and expresses the anti-CD7 CAR.

28. A recombinant retroviral vector comprising:
(a) a first promoter element operably linked to a first polynucleotide encoding an anti-CD7 chimeric antigen receptor (CAR) comprising at least 90% sequence identity to the amino acid sequence of SEQ ID NO:28 or SEQ ID NO:30; and
(b) a second promoter element operably linked to a second polynucleotide encoding an anti-CD7 protein expression blocker (PEBL) comprising at least 90% sequence identity to the amino acid sequence of SEQ ID NO:24 or SEQ ID NO:26.

29. The recombinant retroviral vector of claim 28, wherein the anti-CD7 CAR comprises the amino acid sequence of SEQ ID NO:28 or SEQ ID NO:30.

30. The recombinant retroviral vector of claim 28 or 29, wherein the anti-CD7 PEBL comprises the amino acid sequence of SEQ ID NO:24 or SEQ ID NO:26.

31. The recombinant retroviral vector of any one of claims 28 to 30, wherein the first promoter element and/or the second promoter element are selected from the group consisting of a CMV promoter, EF1α promoter, EFS promoter, MSCV promoter, and PGK promoter.

32. The recombinant retroviral vector of any one of claims 28 to 31, wherein the first promoter element and/or the second promoter element comprise at least 90% sequence identity to the nucleic acid sequence of any one selected from the group consisting of SEQ ID NOS:6-10.

33. The recombinant retroviral vector of any one of claims 28 to 32, wherein the first promoter element and/or the second promoter element comprise the nucleic acid sequence of any one selected from the group consisting of SEQ ID NOS:6-10.

34. The recombinant retroviral vector of any one of claims 28 to 33, wherein the first promoter and the second promoter share less than 95% sequence identity.

35. The recombinant retroviral vector of any one of claims 28 to 34, wherein the first promoter element operably linked to the first polynucleotide is 5′ of the second promoter element operably linked to the second polynucleotide.

36. The recombinant retroviral vector of any one of claims 28 to 34, wherein the second promoter element operably linked to the second polynucleotide is 5′ of the first promoter element operably linked to the first polynucleotide.

37. The recombinant retroviral vector of any one of claims 28 to 35, comprising at least 90% sequence identity to the nucleic acid sequence of any one selected from the group consisting of SEQ ID NOS:18-23.

38. The recombinant retroviral vector of any one of claims 28 to 37, wherein the retroviral vector is a lentiviral vector.

39. An engineered immune cell comprising the recombinant retroviral vector of any one of claims 28 to 38.

40. The engineered immune cell of claim 39, wherein the engineered immune cell is an allogenic T cell.

41. The engineered immune cell of claim 40, wherein the engineered immune cell is an autologous T cell.

42. The engineered immune cell of claim 40, wherein the engineered immune cell has reduced CD7 surface expression compared to a corresponding immune cell and expresses the anti-CD7 CAR.

43. A pharmaceutical composition comprising the engineered immune cell of any one of claim 39 to 42 and a pharmaceutically effective carrier.

44. A method of treating a cancer in a subject comprising administering therapeutically effective amount of the engineered immune cell of any one of claims 39 to 41, or the pharmaceutical composition of claim 43.

45. A method of producing an engineered immune cell comprising transducing an immune cell with the recombinant retroviral vector of any one of claims 28 to 38, and recovering the engineered immune cell.

46. The method of claim 45, wherein the immune cell is selected from the group consisting of a peripheral blood mononuclear cell, an isolated CD4+ T cell, an isolated CD8+ T cell, and an isolated CD3+ T cell.

47. The method of claim 45 or 46, wherein the engineered immune cell has reduced CD7 surface expression compared to a corresponding immune cell and expresses the anti-CD7 CAR.


SEQUENCE LISTING (98 sequences)

SEQ ID NO: 1 - DNA, 2708 bp, Artificial Sequence
  synthetic polynucleotide of CPPT-CMV-MCS-PGK- GFP-WPRE
atcgattttaaaagaaaaggggggattggggggtacagtgcaggggaaagaatagtagacataatagcaacagacatacaaactaaagaattacaaaaacaaattacaaaaattcaaaattttcgggtttattaattaacagggacatgcagagatccagtttaagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatcgctagcgctaccggactcagatcgaattcctcaagcttcctcgagtgcagtcgacgcgggcccgggatcccgcgactctagataattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgactggccaccggcttccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgg

SEQ ID NO: 2 - DNA, 855 bp, Artificial Sequence
  synthetic polynucleotide of anti-human CD7 ( TH69) PEBL
atggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 3 - DNA, 846 bp, Artificial Sequence
  synthetic polynucleotide of anti-human CD7 (3A1F) PEBL
atggctctgcccgtcaccgctctgctgctgcctctggctctgctgctgcacgccgcaagaccccaggtccagctgcaggagtcaggggcagagctggtgaaacccggagccagtgtcaaactgtcctgtaaggccagcggctatactttcaccagctactggatgcactgggtgaaacagaggccaggacagggcctggagtggatcggcaagattaaccccagcaatgggcgcaccaactacaacgaaaagtttaaatccaaggctacactgactgtggacaagagctcctctaccgcatacatgcagctgagttcactgacatctgaagatagtgccgtgtactattgcgccagaggcggggtctactatgacctgtactattacgcactggattattgggggcagggaaccacagtgactgtcagctccggaggaggaggatccggcggaggaggctctgggggaggcgggagtgacatcgagctgacacagtctccagccactctgagcgtgacccctggcgattctgtcagtctgtcatgtagagctagccagtccatctctaacaatctgcactggtaccagcagaaatcacatgaaagccctcggctgctgattaagagtgcttcacagagcatctccgggattccaagcagattctctggcagtgggtcaggaaccgactttacactgtccattaactctgtggagaccgaagatttcggcatgtatttttgccagcagagcaattcctggccttacacattcggaggcgggactaaactggagattaagagggagcagaaactgattagcgaagaggacctgaaggacgaactgtga

SEQ ID NO: 4 - DNA, 1482 bp, Artificial Sequence
  synthetic polynucleotide of anti-human CD7 (TH69) CAR
atggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatga

SEQ ID NO: 5 - DNA, 1476 bp, Artificial Sequence
  synthetic polynucleotide of anti-human CD7 (3A1F) CAR
atggctctgcccgtcaccgctctgctgctgcctctggctctgctgctgcacgctgctcgaccacaggtccagctgcaggagtcaggagctgagctggtgaagccaggggcaagcgtcaaactgtcctgcaaggcctctggatatacattcactagctactggatgcactgggtgaaacagagacccggacagggcctggagtggatcggaaagattaaccctagcaatggcaggaccaactacaacgaaaagtttaaatccaaggcaaccctgacagtggacaagagctcctctacagcctacatgcagctgagttcactgacttcagaggatagcgcagtgtactattgcgccagaggcggggtctactatgacctgtactattacgccctggattattgggggcagggaaccacagtgactgtcagctccggaggaggaggaagcggaggaggaggatccggaggaggaggatctgacatcgagctgacccagagtcctgctacactgagcgtgactccaggcgattctgtcagtctgtcatgtcgggcaagccagtccatctctaacaatctgcactggtaccagcagaaatcccatgaatctccacgactgctgattaagagtgcctcacagagcatctccggcattccctcccggttctctggcagtgggtcaggaactgactttaccctgagtattaactcagtggagacagaagatttcggcatgtatttttgccagcagagcaattcctggccctacactttcggaggcgggaccaaactggagatcaagcggactaccacaccagctccaagaccacctacccctgcaccaacaattgctagtcagccactgtcactgagaccagaagcatgtaggcctgcagctggaggagctgtgcacaccagaggcctggactttgcctgcgatatctacatttgggctcctctggcaggaacctgtggcgtgctgctgctgtctctggtcatcacactgtattgcaaacgagggcggaagaaactgctgtacattttcaagcagccctttatgaggcctgtgcagactacccaggaggaagacggctgcagctgtcgcttccctgaggaagaggaagggggatgtgagctgcgcgtcaaattttctcgaagtgcagatgccccagcttatcagcagggccagaaccagctgtacaacgagctgaatctggggcggagagaggaatatgacgtgctggataagaggcgaggaagggacccagaaatgggagggaaaccacgacggaagaacccccaggagggcctgtataatgaactgcagaaagacaagatggcagaggcctactctgaaatcgggatgaaaggagagagaaggcgcggcaaggggcatgatggcctgtaccaggggctgagcacagcaaccaaagacacctatgacgcactgcacatgcaggcactgccaccacggtaatga

SEQ ID NO: 6 - DNA, 255 bp, Artificial Sequence
  synthetic polynucleotide of CMV promoter
aagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagt

SEQ ID NO: 7 - DNA, 1179 bp, Artificial Sequence
  synthetic polynucleotide of EF1a promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 8 - DNA, 373 bp, Artificial Sequence
  synthetic polynucleotide of EFS promoter
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctag

SEQ ID NO: 9 - DNA, 384 bp, Artificial Sequence
  synthetic polynucleotide of MSCV promoter
ggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcc

SEQ ID NO: 10 - DNA, 509 bp, Artificial Sequence
  synthetic polynucleotide of PGK promoter
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcga

SEQ ID NO: 11 - DNA, 2974 bp, Artificial Sequence
  synthetic polynucleotide of anti-human CD7 (TH69) PEBL-IRES-anti-human CD7 (TH69) CAR
atggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaacgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgacagccaggggatttcaccactcaaaggccagacctgcagacgcccagattatgagacaca

SEQ ID NO: 12 - DNA, 2974 bp, Artificial Sequence
  synthetic polynucleotide of anti-human CD7 (TH69) CAR-IRES-anti-human CD7 (TH69) PEBL
atggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgacagccaggggatttcaccactcaaaggccagacctgcagacgcccagattatgagacacacgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 13 - DNA, 2400 bp, Artificial Sequence
  synthetic polynucleotide of anti-human CD7 (TH69) CAR-p2A-anti-human CD7 (TH69) PEBL
atggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagaggcagcggcgccacaaacttctctctgctaaagcaagcaggtgatgttgaagaaaaccccgggcctatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 14 - DNA, 2803 bp, Artificial Sequence
  synthetic polynucleotide of MSCV promoter- Anti-human CD7 (TH69) CAR-p2A-Anti-human CD7 (TH69) PEBL
ggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtccgaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgaggcagcggcgccacaaacttctctctgctaaagcaagcaggtgatgttgaagaaaaccccgggcctatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 15 - DNA, 3598 bp, Artificial Sequence
  synthetic polynucleotide of EF1a promoter- Anti-human CD7 (TH69) CAR-p2A-Anti-human CD7 (TH69) PEBL
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgaggcagcggcgccacaaacttctctctgctaaagcaagcaggtgatgttgaagaaaaccccgggcctatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 16 - DNA, 2792 bp, Artificial Sequence
  synthetic polynucleotide of EF1a promoter- Anti-human CD7 (TH69) CAR-p2A-Anti-human CD7 (TH69) PEBL
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgaggcagcggcgccacaaacttctctctgctaaagcaagcaggtgatgttgaagaaaaccccgggcctatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 17 - DNA, 3346 bp, Artificial Sequence
  synthetic polynucleotide of MSCV promoter- Anti-human CD7 (TH69) CAR-PGK promoter-Anti-human CD7 (TH69) PEBL
ttaattaatttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtccgaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgactcgagtgcagtcgacgcgggcccgggatcccgcgactctagataattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgactggccaggcttccaccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaacgcgt

SEQ ID NO: 18 - DNA, 3938 bp, Artificial Sequence
  synthetic polynucleotide of MSCV promoter- anti-human CD7 CAR (TH69)-EF1a promoter- Anti-human CD7
ggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtccgaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgagtcgacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccgggggcttccaccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 19 - DNA, 3132 bp, Artificial Sequence
  synthetic polynucleotide of MSCV promoter- anti-human CD7 CAR (TH69)-EFS promoter- Anti-human CD7 (TH69) PEBL
ggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtccgaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgagtcgacgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctagcccgggggcttccaccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 20 - DNA, 4057 bp, Artificial Sequence
  synthetic polynucleotide of PGK promoter-anti- human CD7 CAR (3A1F)-EF1a promoter- Anti-human CD7 (TH69) PEBL
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgagaattcggcttccaccatggctctgcccgtcaccgctctgctgctgcctctggctctgctgctgcacgctgctcgaccacaggtccagctgcaggagtcaggagctgagctggtgaagccaggggcaagcgtcaaactgtcctgcaaggcctctggatatacattcactagctactggatgcactgggtgaaacagagacccggacagggcctggagtggatcggaaagattaaccctagcaatggcaggaccaactacaacgaaaagtttaaatccaaggcaaccctgacagtggacaagagctcctctacagcctacatgcagctgagttcactgacttcagaggatagcgcagtgtactattgcgccagaggcggggtctactatgacctgtactattacgccctggattattgggggcagggaaccacagtgactgtcagctccggaggaggaggaagcggaggaggaggatccggaggaggaggatctgacatcgagctgacccagagtcctgctacactgagcgtgactccaggcgattctgtcagtctgtcatgtcgggcaagccagtccatctctaacaatctgcactggtaccagcagaaatcccatgaatctccacgactgctgattaagagtgcctcacagagcatctccggcattccctcccggttctctggcagtgggtcaggaactgactttaccctgagtattaactcagtggagacagaagatttcggcatgtatttttgccagcagagcaattcctggccctacactttcggaggcgggaccaaactggagatcaagcggactaccacaccagctccaagaccacctacccctgcaccaacaattgctagtcagccactgtcactgagaccagaagcatgtaggcctgcagctggaggagctgtgcacaccagaggcctggactttgcctgcgatatctacatttgggctcctctggcaggaacctgtggcgtgctgctgctgtctctggtcatcacactgtattgcaaacgagggcggaagaaactgctgtacattttcaagcagccctttatgaggcctgtgcagactacccaggaggaagacggctgcagctgtcgcttccctgaggaagaggaagggggatgtgagctgcgcgtcaaattttctcgaagtgcagatgccccagcttatcagcagggccagaaccagctgtacaacgagctgaatctggggcggagagaggaatatgacgtgctggataagaggcgaggaagggacccagaaatgggagggaaaccacgacggaagaacccccaggagggcctgtataatgaactgcagaaagacaagatggcagaggcctactctgaaatcgggatgaaaggagagagaaggcgcggcaaggggcatgatggcctgtaccaggggctgagcacagcaaccaaagacacctatgacgcactgcacatgcaggcactgccaccacggtaatgagtcgacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccgggggcttccaccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 21 - DNA, 4054 bp, Artificial Sequence
  synthetic polynucleotide of PGK promoter-anti- human CD7 CAR (TH69)-EF1a promoter- Anti-human CD7 (3A1F) PEBL
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgagaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgagtcgacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccgggggcttccaccatggctctgcccgtcaccgctctgctgctgcctctggctctgctgctgcacgccgcaagaccccaggtccagctgcaggagtcaggggcagagctggtgaaacccggagccagtgtcaaactgtcctgtaaggccagcggctatactttcaccagctactggatgcactgggtgaaacagaggccaggacagggcctggagtggatcggcaagattaaccccagcaatgggcgcaccaactacaacgaaaagtttaaatccaaggctacactgactgtggacaagagctcctctaccgcatacatgcagctgagttcactgacatctgaagatagtgccgtgtactattgcgccagaggcggggtctactatgacctgtactattacgcactggattattgggggcagggaaccacagtgactgtcagctccggaggaggaggatccggcggaggaggctctgggggaggcgggagtgacatcgagctgacacagtctccagccactctgagcgtgacccctggcgattctgtcagtctgtcatgtagagctagccagtccatctctaacaatctgcactggtaccagcagaaatcacatgaaagccctcggctgctgattaagagtgcttcacagagcatctccgggattccaagcagattctctggcagtgggtcaggaaccgactttacactgtccattaactctgtggagaccgaagatttcggcatgtatttttgccagcagagcaattcctggccttacacattcggaggcgggactaaactggagattaagagggagcagaaactgattagcgaagaggacctgaaggacgaactgtga

SEQ ID NO: 22 - DNA, 4063 bp, Artificial Sequence
  synthetic polynucleotide of PGK promoter-anti- human CD7 CAR (TH69)-EF1a promoter-Anti-human CD7 (TH69) PEBL
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgagaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgagtcgacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacccgggggcttccaccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 23 - DNA, 3257 bp, Artificial Sequence
  synthetic polynucleotide of PGK promoter-anti- human CD7 CAR (TH69)-EFS promoter-Anti-human CD7 (TH69) PEBL
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgagaattcggcttccaccatggctctgcctgtgactgccctgctgctgcccctggccctgctgctgcacgctgctagacccgccgcatacaaggacattcagatgactcagactacatccagcctgtcagcttccctgggcgatcgagtgactatcagctgttcagcaagccaggggatcagcaattacctgaactggtaccagcagaagcccgacggaactgtcaagctgctgatctactatacttccagtctgcatagcggggtccctagccgattctctgggagtggaagcggcaccgactatagcctgaccatttccaacctggaaccagaggacattgccacctattactgtcagcagtattcaaagctgccctatacctttggcggagggactaagctggagatcaagcgaggagggggaggatcagggggaggggggagcggaggcggggggagtggcggcggagggtcagaagtccagctggtcgagagtgggggcggactggtgaaacctgggggctctctgaaactgagctgtgcagccagtggcctgacattctcctcatatgctatgtcttgggtgcggcagaccccagaaaagagactggagtgggtggcttcaatctcttccgggggcttcacttactatcctgacagcgtgaaaggcagattcactatctccagggataacgcacggaatatcctgtatctgcagatgagctcactgagatccgaggacaccgcaatgtactactgcgcaagagacgaagtgcgggggtatctggacgtgtggggagcaggcactaccgtcaccgtgtctagcaccactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgcaagcgggggcgcaaaaaactgctgtatatctttaagcagcctttcatgagaccagtgcagacaacccaggaggaagatgggtgctcatgccggtttcccgaggaggaggaaggcggctgcgagctgagggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccagatgagtcgacgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctagcccgggggcttccaccatggctctgcctgtgaccgcactgctgctgcccctggctctgctgctgcacgccgcaagacctgccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaaggggaggaggaggaagcggaggaggaggatccggaggcgggggatctggaggaggaggaagtgaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagcgagcagaaactgattagcgaagaggacctgaaagatgaactgtaa

SEQ ID NO: 24 - protein, 284 aa, Artificial Sequence
  synthetic polypeptide of anti CD7 (TH69) PEBL
MALPVTALLLPLALLLHAARPAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSEQKLISEEDLKDEL

SEQ ID NO: 25 - protein, 285 aa, Artificial Sequence
  synthetic polypeptide of anti CD7 (TH69) PEBL variant
PMALPVTALLLPLALLLHAARPAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSEQKLISEEDLKDEL

SEQ ID NO: 26 - protein, 281 aa, Artificial Sequence
  synthetic polypeptide of anti-human CD7 (3A1F) PEBL
MALPVTALLLPLALLLHAARPQVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSSGGGGSGGGGSGGGGSDIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKREQKLISEEDLKDEL

SEQ ID NO: 27 - protein, 282 aa, Artificial Sequence
  synthetic polypeptide of anti-human CD7 (3A1F) PEBL variant
PMALPVTALLLPLALLLHAARPQVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSSGGGGSGGGGSGGGGSDIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKREQKLISEEDLKDEL

SEQ ID NO: 28 - protein, 493 aa, Artificial Sequence
  synthetic polypeptide of anti-human CD7 (TH69) CAR
MALPVTALLLPLALLLHAARPAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 514 aa, Artificial Sequence
  synthetic polypeptide of anti-human CD7 (TH69) CAR variant
MALPVTALLLPLALLLHAARPAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 30 - protein, 490 aa, Artificial Sequence
  synthetic polypeptide of anti-human CD7 (3A1F) CAR
MALPVTALLLPLALLLHAARPQVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSSGGGGSGGGGSGGGGSDIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 511 aa, Artificial Sequence
  synthetic polypeptide of anti-human CD7 (3A1F) CAR variant
MALPVTALLLPLALLLHAARPQVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSSGGGGSGGGGSGGGGSDIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 32 - protein, 117 aa, Artificial Sequence
  synthetic polypeptide of anti-human CD7 TH69 VH domain
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSS

SEQ ID NO: 33 - protein, 112 aa, Artificial Sequence
  synthetic polypeptide of anti-human CD7 TH69 VL domain
AAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKR

SEQ ID NO: 34 - protein, 123 aa, Artificial Sequence
  synthetic polypeptide of anti-human CD7 3A1F VH domain
QVQLQESGAELVKPGASVKLSCKASGYTFTSYWMHWVKQRPGQGLEWIGKINPSNGRTNYNEKFKSKATLTVDKSSSTAYMQLSSLTSEDSAVYYCARGGVYYDLYYYALDYWGQGTTVTVSS

SEQ ID NO: 35 - protein, 108 aa, Artificial Sequence
  synthetic polypeptide of anti-human CD7 3A1F VL domain
DIELTQSPATLSVTPGDSVSLSCRASQSISNNLHWYQQKSHESPRLLIKSASQSISGIPSRFSGSGSGTDFTLSINSVETEDFGMYFCQQSNSWPYTFGGGTKLEIKR

SEQ ID NO: 36 - protein, 120 aa, Artificial Sequence
  synthetic polypeptide of anti-human CD7 T3-3A1 VH domain
DVQLVESGGGLVQPGGSRKLSCAASGFTFSSFGMHWVRQAPEKGLEWVAYISSGSSTLHYADTVKGRFTISRDNPKNTLFLQMTSLRSEDTAMYYCARWGNYPHYAMDYWGQGTSVTVSS

SEQ ID NO: 37 - protein, 111 aa, Artificial Sequence
  synthetic polypeptide of anti-human CD7 T3-3A1 VL domain
DIVMTQSPASLAVSLGQRATISCRASKSVSASGYSYMHWYQQKPGQPPKLLIYLASNLESGVPARFSGSGSGTDFTLNIHPVEEEDAVTYYCQHSRELPYTFGGGTKLEIK

SEQ ID NO: 38 - DNA, 351 bp, Artificial Sequence
  synthetic polynucleotide of anti-human CD7 TH69 VH domain
gaggtgcagctggtcgaatctggaggaggactggtgaagccaggaggatctctgaaactgagttgtgccgcttcaggcctgaccttctcaagctacgccatgagctgggtgcgacagacacctgagaagcggctggaatgggtcgctagcatctcctctggcgggttcacatactatccagactccgtgaaaggcagatttactatctctcgggataacgcaagaaatattctgtacctgcagatgagttcactgaggagcgaggacaccgcaatgtactattgtgccagggacgaagtgcgcggctatctggatgtctggggagctggcactaccgtcaccgtctccagc

SEQ ID NO: 39 - DNA, 336 bp, Artificial Sequence
  synthetic polynucleotide of anti-human CD7 TH69 VL domain
gccgcatacaaggatattcagatgactcagaccacaagctccctgagcgcctccctgggagaccgagtgacaatctcttgcagtgcatcacagggaattagcaactacctgaattggtatcagcagaagccagatggcactgtgaaactgctgatctactatacctctagtctgcacagtggggtcccctcacgattcagcggatccggctctgggacagactacagcctgactatctccaacctggagcccgaagatattgccacctactattgccagcagtactccaagctgccttatacctttggcgggggaacaaagctggagattaaaagg

SEQ ID NO: 40 - DNA, 369 bp, Artificial Sequence
  synthetic polynucleotide of anti-human CD7 3A1F VH domain
caggtccagctgcaggagtcaggagctgagctggtgaagccaggggcaagcgtcaaactgtcctgcaaggcctctggatatacattcactagctactggatgcactgggtgaaacagagacccggacagggcctggagtggatcggaaagattaaccctagcaatggcaggaccaactacaacgaaaagtttaaatccaaggcaaccctgacagtggacaagagctcctctacagcctacatgcagctgagttcactgacttcagaggatagcgcagtgtactattgcgccagaggcggggtctactatgacctgtactattacgccctggattattgggggcagggaaccacagtgactgtcagctcc

SEQ ID NO: 41 - DNA, 324 bp, Artificial Sequence
  synthetic polynucleotide of anti-human CD7 3A1F VL domain
gacatcgagctgacccagagtcctgctacactgagcgtgactccaggcgattctgtcagtctgtcatgtcgggcaagccagtccatctctaacaatctgcactggtaccagcagaaatcccatgaatctccacgactgctgattaagagtgcctcacagagcatctccggcattccctcccggttctctggcagtgggtcaggaactgactttaccctgagtattaactcagtggagacagaagatttcggcatgtatttttgccagcagagcaattcctggccctacactttcggaggcgggaccaaactggagatcaagcgg

SEQ ID NO: 42 - DNA, 360 bp, Artificial Sequence
  synthetic polynucleotide of anti-human CD7 T3-3A1 VH domain
gatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacattagtagtggcagtagtaccctccactatgcagacacagtgaagggccgattcaccatctccagagacaatcccaagaacaccctgttcctgcaaatgaccagtctaaggtctgaggacacggccatgtattactgtgcaagatggggtaactaccctcactatgctatggactactggggtcaaggaacctcagtcaccgtctcctca

SEQ ID NO: 43 - DNA, 333 bp, Artificial Sequence
  synthetic polynucleotide of anti-human CD7 T3-3A1 VL domain
gacattgtgatgacccagtctcctgcttccttagctgtatctctggggcagagggccaccatctcatgcagggccagcaaaagtgtcagtgcatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgtaacctattactgtcagcacagtagggagcttccgtacacgttcggaggggggaccaagctggaaataaaa

SEQ ID NO: 44 - protein, 11 aa, Artificial Sequence
  synthetic peptide
SASQGISNYLN

SEQ ID NO: 45 - protein, 7 aa, Artificial Sequence
  synthetic peptide
YTSSLHS

SEQ ID NO: 46 - protein, 9 aa, Artificial Sequence
  synthetic peptide
QQYSKLPYT

SEQ ID NO: 47 - protein, 5 aa, Artificial Sequence
  synthetic peptide
SYAMS

SEQ ID NO: 48 - protein, 16 aa, Artificial Sequence
  synthetic peptide
SISSGGFTYYPDSVKG

SEQ ID NO: 49 - protein, 9 aa, Artificial Sequence
  synthetic peptide
DEVRGYLDV

SEQ ID NO: 50 - protein, 11 aa, Artificial Sequence
  synthetic peptide
RASQSISNNLH

SEQ ID NO: 51 - protein, 7 aa, Artificial Sequence
  synthetic peptide
SASQSIS

SEQ ID NO: 52 - protein, 9 aa, Artificial Sequence
  synthetic peptide
QQSNSWPYT

SEQ ID NO: 53 - protein, 5 aa, Artificial Sequence
  synthetic peptide
SYWMH

SEQ ID NO: 54 - protein, 17 aa, Artificial Sequence
  synthetic peptide
KINPSNGRTNYNEKFKS

SEQ ID NO: 55 - protein, 14 aa, Artificial Sequence
  synthetic peptide
GGVYYDLYYYALDY

SEQ ID NO: 56 - protein, 15 aa, Artificial Sequence
  synthetic peptide
RASKSVSASGYSYMH

SEQ ID NO: 57 - protein, 7 aa, Artificial Sequence
  synthetic peptide
LASNLES

SEQ ID NO: 58 - protein, 9 aa, Artificial Sequence
  synthetic peptide
QHSRELPYT

SEQ ID NO: 59 - protein, 5 aa, Artificial Sequence
  synthetic peptide
SFGMH

SEQ ID NO: 60 - protein, 17 aa, Artificial Sequence
  synthetic peptide
YISSGSSTLHYADTVKG

SEQ ID NO: 61 - protein, 11 aa, Artificial Sequence
  synthetic peptide
WGNYPHYAMDY

SEQ ID NO: 62 - DNA, 577 bp, Artificial Sequence
  synthetic polynucleotide of IRES
cgggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataatacc

SEQ ID NO: 63 - DNA, 57 bp, Artificial Sequence
  synthetic polynucleotide of P2A
gccacaaacttctctctgctaaagcaagcaggtgatgttgaagaaaaccccgggcct

SEQ ID NO: 64 - DNA, 60 bp, Artificial Sequence
  synthetic polynucleotide of E2A
cagtgtactaattatgctctcttgaaattggctggagatgttgagagcaacggaggtccc

SEQ ID NO: 65 - DNA, 66 bp, Artificial Sequence
  synthetic polynucleotide of F2A
gtgaaacagactttgaattttgaccttctcaagttggcgggagacgtggagtccaaccctggacct

SEQ ID NO: 66 - DNA, 54 bp, Artificial Sequence
  synthetic polynucleotide of T2A
gagggcaggggaagtcttctaacatgcggggacgtggaggaaaatcccggccca

SEQ ID NO: 67 - protein, 19 aa, Artificial Sequence
  synthetic polypeptide of P2A
ATNFSLLKQAGDVEENPGP

SEQ ID NO: 68 - protein, 20 aa, Artificial Sequence
  synthetic polypeptide of E2A
QCTNYALLKLAGDVESNPGP

SEQ ID NO: 69 - protein, 22 aa, Artificial Sequence
  synthetic polypeptide of F2A
VKQTLNFDLLKLAGDVESNPGP

SEQ ID NO: 70 - protein, 18 aa, Artificial Sequence
  synthetic polypeptide of T2A
EGRGSLLTCGDVEENPGP

SEQ ID NO: 71 - protein, 14 aa, Artificial Sequence
  synthetic polypeptide of myc-KDEL domain
EQKLISEEDLKDEL

SEQ ID NO: 72 - protein, 26 aa, Artificial Sequence
  synthetic polypeptide of link(20)AEKDEL domain
GGGGSGGGGSGGGGSGGGGSAEKDEL

SEQ ID NO: 73 - protein, 4 aa, Artificial Sequence
  synthetic polypeptide of KDEL domain
KDEL

SEQ ID NO: 74 - protein, 4 aa, Artificial Sequence
  synthetic polypeptide of KXD/E domain
  MISC_FEATURE (3)..(3): wherein Xaa is any amino acid
  MISC_FEATURE (4)..(4): wherein Xaa is any amino acid
KKXX

SEQ ID NO: 75 - protein, 4 aa, Artificial Sequence
  synthetic polypeptide of YQRL domain
YQRL

SEQ ID NO: 76 - protein, 4 aa, Artificial Sequence
  synthetic polypeptide of PEST motif
PEST

SEQ ID NO: 77 - protein, 83 aa, Artificial Sequence
  synthetic polypeptide of mb-DEKKMP domain
TTTPAPRPPTPAPTIASQPLSLRPEACRPAAGGAVHTRGLDFACDIYIWAPLAGTCGVLLLSLVITLYKYKSRRSFIDEKKMP

SEQ ID NO: 78 - protein, 68 aa, Artificial Sequence
  synthetic polypeptide of CD8alpha hinge and transmembrane domain
TTTPAPRPPTPAPTIASQPLSLRPEACRPAAGGAVHTRGLDFACDIYIWAPLAGTCGVLLLSLVITLY

SEQ ID NO: 79 - protein, 20 aa, Artificial Sequence
  synthetic polypeptide of VH-VL linker
GGGGSGGGGSGGGGSGGGGS

SEQ ID NO: 80 - protein, 21 aa, Artificial Sequence
  synthetic polypeptide of CD8alpha signal peptide
MALPVTALLLPLALLLHAARP

SEQ ID NO: 81 - DNA, 63 bp, Artificial Sequence
  synthetic polynucleotide of CD8alpha signal peptide
atggctctgcctgtgaccgcactgctgctgcccctggctctgctgctgcacgccgcaagacct

SEQ ID NO: 82 - DNA, 60 bp, Artificial Sequence
  synthetic polynucleotide of VH-VL linker
ggaggaggaggaagcggaggaggaggatccggaggcgggggatctggaggaggaggaagt

SEQ ID NO: 83 - DNA, 42 bp, Artificial Sequence
  synthetic polynucleotide of myc-KDEL domain
gagcagaaactgattagcgaagaggacctgaaagatgaactg

SEQ ID NO: 84 - protein, 68 aa, Artificial Sequence; identical to SEQ ID NO: 78
  synthetic polypeptide of CD8alpha hinge and transmembrane domain

SEQ ID NO: 85 - protein, 42 aa, Artificial Sequence
  synthetic polypeptide of intracellular signaling domain of 4-1BB
KRGRKKLLYIFKQPFMRPVQTTQEEDGCSCRFPEEEEGGCEL

SEQ ID NO: 86 - protein, 112 aa, Artificial Sequence
  synthetic polypeptide of intracellular signaling domain CD3zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - DNA, 20 bp, Artificial Sequence
  synthetic oligonucleotide
gagggaagctcatcagtggg

SEQ ID NO: 88 - DNA, 20 bp, Artificial Sequence
  synthetic oligonucleotide
catctcctgcccagtctgac

SEQ ID NO: 89 - DNA, 21 bp, Artificial Sequence
  synthetic oligonucleotide
cctttccgggactttcgcttt

SEQ ID NO: 90 - DNA, 20 bp, Artificial Sequence
  synthetic oligonucleotide
gcagaatccaggtggcaaca

SEQ ID NO: 91 - DNA, 20 bp, Artificial Sequence
  synthetic oligonucleotide
gcagcctttcatgagaccag

SEQ ID NO: 92 - DNA, 20 bp, Artificial Sequence
  synthetic oligonucleotide
tgcccaggttcagctcatta

SEQ ID NO: 93 - DNA, 20 bp, Artificial Sequence
  synthetic oligonucleotide
acctgccgcatacaaggata

SEQ ID NO: 94 - DNA, 20 bp, Artificial Sequence
  synthetic oligonucleotide
ccactgtgcagactagaggt

SEQ ID NO: 95 - protein, 799 aa, Artificial Sequence
  synthetic polypeptide of anti-human CD7 (TH69) CAR-p2A- Anti-human CD7 (TH69) PEBL
MALPVTALLLPLALLLHAARPAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSEQKLISEEDLKDEL

SEQ ID NO: 96 - DNA, 207 bp, Artificial Sequence
  synthetic polypeptide of CD8alpha hinge and transmembrane domain
accactacacctgcaccaaggcctcccacacccgctcccactatcgcttcccagccactgtccctgaggcccgaggcctgcaggccagcagctggcggagccgtgcatactagggggctggacttcgcttgcgacatctacatctgggccccactggcagggacatgcggagtcctgctgctgtccctggtcatcacactgtactgc

SEQ ID NO: 97 - DNA, 126 bp, Artificial Sequence
  synthetic polypeptide of intracellular signaling domain of 4-1BB
aagcgggggcgcaaaaaactgctgtatatctttaagcagcctttcatgagaccagtgcagacaacccaggaggaagatgggtgctcatgccggtttcccgaggaggaggaaggcggctgcgagctg

SEQ ID NO: 98 - DNA, 335 bp, Artificial Sequence
  synthetic polypeptide of intracellular signaling domain CD3zeta
gggtgaagttttcccgctcagcagatgctcctgcctaccagcagggccagaaccagctgtataatgagctgaacctgggcagacgcgaagagtatgatgtgctggacaaaaggcggggaagagaccccgaaatgggagggaagccaaggcggaaaaacccccaggagggcctgtacaatgagctgcagaaggacaaaatggcagaggcttacagtgagattgggatgaagggagagagacggaggggaaaagggcacgatggcctgtaccaggggctgagcacagcaaccaaagatacttatgacgcactgcacatgcaggcactgccacccaga